(19) United States
(12) Patent Application Publication
(10) Pub. No.: US 2023/0219906 A1
(43) Pub. Date: Jul. 13, 2023

(54) MACROCYCLIC-2-AMINO-3-FLUORO-BUT-3-ENAMIDES AS INHIBITORS OF MCL-1

(71) Applicant: Janssen Pharmaceutica NV, Beerse (BE)

(72) Inventors: Soufyan JERHAOUI, Beerse (BE); Frederik Jan Rita ROMBOUTS, Beerse (BE); Gaston Stanislas Marcella DIELS, Beerse (BE); Michel SURKYN, Beerse (BE); Matthieu Dominique JOUFFROY, Beerse (BE)

(21) Appl. No.: 18/000,439

(22) PCT Filed: Jun. 9, 2021
(86) PCT No.: PCT/EP2021/065483

§ 371 Date: Dec. 1, 2022

(30) Foreign Application Priority Data
Jun. 10, 2020 (EP) 20179237.1

(51) Int. Cl. C07D 267/16; A61P 35/00

(52) U.S. Cl. CPC C07D 267/16; A61P 35/00

(57) ABSTRACT

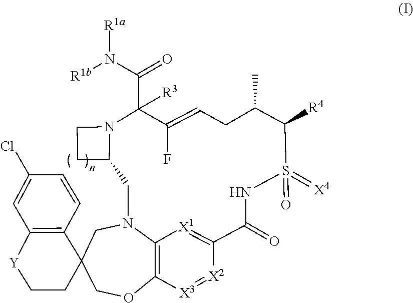

The present invention relates to pharmaceutical agents useful for therapy and/or prophylaxis in a subject, pharmaceutical composition comprising such compounds (formula (I)), and their use as MCL-1 inhibitors, useful for treating diseases such as cancer.

### FIELD OF THE INVENTION

The present invention relates to pharmaceutical agents useful for therapy and/or prophylaxis in a subject, pharmaceutical composition comprising such compounds, and their use as MCL-1 inhibitors, useful for treating or preventing diseases such as cancer.

### BACKGROUND OF THE INVENTION

Cellular apoptosis or programmed cell death is critical to the development and homeostasis of many organs including the hematopoietic system. Apoptosis can be initiated via the extrinsic pathway, which is mediated by death receptors, or by the intrinsic pathway using the B cell lymphoma (BCL-2) family of proteins. Myeloid cell leukemia-1 (MCL-1) is a member of the BCL-2 family of cell survival regulators and is a critical mediator of the intrinsic apoptosis pathway. MCL-1 is one of five principal anti-apoptotic BCL-2 proteins (MCL-1, BCL-2, BCL-XL, BCL-w, and BFL1/A1) responsible for maintaining cell survival. MCL-1 continuously and directly represses the activity of the pro-apoptotic BCL-2 family proteins Bak and Bax and indirectly blocks apoptosis by sequestering BH3 only apoptotic sensitizer proteins such as Bim and Noxa. The activation of Bak/Bax following various types of cellular stress leads to aggregation on the mitochondrial outer membrane and this aggregation facilitates pore formation, loss of mitochondrial outer membrane potential, and subsequent release of cytochrome C into the cytosol. Cytosolic cytochrome C binds Apaf-1 and initiates recruitment of procaspase 9 to form apoptosome structures (Cheng et al. eLife 2016; 5: e17755). The assembly of apoptosomes activates the executioner cysteine proteases 3/7 and these effector caspases then cleave a variety of cytoplasmic and nuclear proteins to induce cell death (Julian et al. Cell Death and Differentiation 2017; 24, 1380-1389).

Avoiding apoptosis is an established hallmark of cancer development and facilitates the survival of tumor cells that would otherwise be eliminated due to oncogenic stresses, growth factor deprivation, or DNA damage (Hanahan and Weinberg. Cell 2011; 1-44). Thus, unsurprisingly, MCL-1 is highly upregulated in many solid and hematologic cancers relative to normal non-transformed tissue counterparts. The overexpression of MCL-1 has been implicated in the pathogenesis of several cancers where it correlated with poor outcome, relapse, and aggressive disease. Additionally, overexpression of MCL-1 has been implicated in the pathogenesis of the following cancers: prostate, lung, pancreatic, breast, ovarian, cervical, melanoma, B-cell chronic lymphocytic leukemia (CLL), acute myeloid leukemia (AML), and acute lymphoblastic leukemia (ALL). The human MCL-1 genetic locus (1q21) is frequently amplified in tumors and quantitatively increases total MCL-1 protein levels (Beroukhim et al. Nature 2010; 463 (7283) 899-905). MCL-1 also mediates resistance to conventional cancer therapeutics and is transcriptionally upregulated in response to inhibition of BCL-2 function (Yecies et al. Blood 2010; 115 (16)3304-3313).

A small molecule BH3 inhibitor of BCL-2 has demonstrated clinical efficacy in patients with chronic lymphocytic leukemia and is FDA approved for patients with CLL or AML (Roberts et al. NEJM 2016; 374:311-322). The clinical success of BCL-2 antagonism led to the development of several MCL-1 BH3 mimetics that show efficacy in preclinical models of both hematologic malignancies and solid tumors (Kotschy et al. Nature 2016; 538 477-486, Merino et al. Sci. Transl. Med; 2017 (9)).

MCL-1 regulates several cellular processes in addition to its canonical role in mediating cell survival including mitochondrial integrity and non-homologous end joining following DNA damage (Chen et al. JCI 2018; 128(1):500-516). The genetic loss of MCL-1 shows a range of phenotypes depending on the developmental timing and tissue deletion. MCL-1 knockout models reveal there are multiple roles for MCL-1 and loss of function impacts a wide range of phenotypes. Global MCL-1-deficient mice display embryonic lethality and studies using conditional genetic deletion have reported mitochondrial dysfunction, impaired activation of autophagy, reductions in B and T lymphocytes, increased B and T cell apoptosis, and the development of heart failure/cardiomyopathy (Wang et al. Genes and Dev 2013; 27 1351-1364, Steimer et al. Blood 2009; (113) 2805-2815).

WO2019046150 discloses macrocyclic compounds that inhibit mcl-1 protein.

WO2016033486 discloses tetrahydronaphthalene derivatives that inhibit mcl-1 protein.

WO2019036575, WO2017147410, and WO2018183418 disclose compounds that inhibit mcl-1 protein.

WO2019222112 discloses MCL-1 inhibitors for treating cancer.

WO2020097577 discloses spiro-sulfonamide derivatives as inhibitors of myeloid cell leukemia-1 (MCL-1) protein.

WO2021021259 describes formulations and dosages for administering a compound that inhibits MCL1 protein.

WO2019173181 discloses MCL-1 inhibitors.

There remains a need for MCL-1 inhibitors, useful for the treatment or prevention of cancers such as prostate, lung, pancreatic, breast, ovarian, cervical, melanoma, B-cell chronic lymphocytic leukemia (CLL), acute myeloid leukemia (AML), and acute lymphoblastic leukemia (ALL).

### SUMMARY OF THE INVENTION

The present invention concerns compounds of Formula (I):

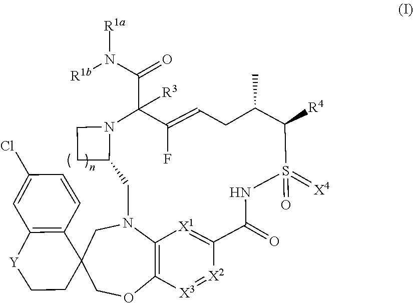

wherein

R^{1a} and R^{1b} are each independently selected from the group consisting of hydrogen, C₁₋₆alkyl, C₂₋₆alkenyl, C₂₋₆alkynyl, C₃₋₇cycloalkyl, C₃₋₇cycloalkenyl, Het¹, Ar¹, Het², and Cy¹, wherein said C₁₋₆alkyl, C₂₋₆alkenyl, C₂₋₆alkynyl, C₃₋₇cycloalkyl or C₃₋₇cycloalkenyl is optionally substituted with one or two R²;

or R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of oxo, OR^{f}, SR^{f}, NR^{d}R^{e}, CN, halo, CF₃, and C₁₋₄alkyl optionally substituted with one substituent selected from the group consisting of ORE, SR^{f}, CN and halo;

or R^{1a} and R^{1b} are taken together to form together with the N-atom to which they are attached a 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of oxo, ORE, SR, NR^{d}R^{e}, CN, halo, CF₃, and C₁₋₄alkyl optionally substituted with one substituent selected from the group consisting of OR^{f}, SR^{f}, CN and halo;

each R² is independently selected from the group consisting of OR^{f}, SR^{f}, CN, halo, CF₃, NR^{m}Rⁿ, SO₂R^{c}, C(═O)R^{c}, C(═O)OR^{d}, C(═O)NR^{d}R^{e}, SO₂NR^{d}R^{e}, C₃₋₇cycloalkyl, C₃₋₇ cycloalkenyl, Het¹, Ar¹, Het², and Cy¹, wherein said C₃₋₇cycloalkyl or C₃₋₇cycloalkenyl is optionally substituted with one or two substituents each independently selected from the group consisting of OR^{f}, SR^{f}, CN, halo and NR^{d}R^{e};

R^{c} is selected from the group consisting of C₁₋₆alkyl, C₃₋₇cycloalkyl, Het¹, Ar¹ and Het²; R^{m} and Rⁿ are each independently selected from the group consisting of hydrogen, methyl, C₂₋₇-alkyl, C₃₋₇cycloalkyl, Het¹, Ar¹, and Het², wherein said C₂₋₇-alkyl or C₃₋₇cycloalkyl is optionally substituted with one or two substituents each independently selected from the group consisting of ORⁱ, SRⁱ, NR^{g}R^{h}, CN, halo, and C₁₋₄alkyl optionally substituted with one substituent selected from the group consisting of ORⁱ, SRⁱ, CN, NR^{g}R^{h} and halo;

R^{d} and R^{e} are each independently selected from the group consisting of hydrogen, methyl, C₂₋₇alkyl, C₃₋₇cycloalkyl, Het¹, Ar¹, and Het², wherein said C₂₋₇alkyl or C₃₋₇cycloalkyl is optionally substituted with one or two substituents each independently selected from the group consisting of ORⁱ, SRⁱ, NR^{g}R^{h}, CN, halo, and C₁₋₄alkyl optionally substituted with one substituent selected from the group consisting of ORⁱ, SRⁱ, CN, NR^{g}R^{h} and halo;

or R^{d} and R^{e} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo; or R^{d} and R^{e} are taken together to form together with the N-atom to which they are attached a fused 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;

n is 1 or 2;

R^{f} is selected from the group consisting of hydrogen, C₁₋₆alkyl, CF₃, C₃₋₇cycloalkyl, Het¹, Ar¹, Het², wherein said C₁₋₆alkyl or C₃₋₇cycloalkyl is optionally substituted with one substituent selected from the group consisting of ORⁱ, SRⁱ, CN, halo, NR^{m}Rⁿ, SO₂R^{c}, C(═O)R^{c}, C(═O)OR^{d}, C(═O)N^{d}R^{e}, SO₂NR^{d}R^{e}, C₃₋₇cycloalkyl, Het¹, Ar¹ and Het²;

R^{g} and R^{h} are each independently selected from the group consisting of hydrogen, C₁₋₆ alkyl and C₃₋₇cycloalkyl;

or R^{g} and R^{h} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂;

Het¹ represents a 4- to 7-membered monocyclic fully saturated heterocyclyl containing one or two heteroatoms each independently selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;

Het² represents a 5- to 6-membered monocyclic aromatic ring containing one, two, three or four heteroatoms each independently selected from O, S, and N, wherein said S-atom might be substituted to form S(═O) or S(═O)₂, and wherein said aromatic ring is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;

Cy¹ represents a 6- to 11-membered bicyclic fully saturated ring system optionally containing one or two heteroatoms each independently selected from O, S, and N, wherein said S-atom might be substituted to form S(═O) or S(═O)₂, and wherein said ring system is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;

Ar¹ represents phenyl optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;

Rⁱ represents hydrogen, C₁₋₆alkyl or C₃₋₇cycloalkyl;

R^{j} and R^{k} are each independently selected from the group consisting of hydrogen, C₁₋₆alkyl and C₃₋₇cycloalkyl;

R³ represents hydrogen, C₁₋₄alkyl or C₁₋₄alkyl-OH;

R⁴ represents hydrogen or methyl;

R⁵ represents —(C═O)-phenyl, —(C═O)-Het⁴ or —(C═O)-Het³; wherein said phenyl, Het³ or

Het⁴ are optionally substituted with one or two substituents selected from methyl or methoxy;

Het⁴ represents a C-linked 4- to 7-membered monocyclic fully saturated heterocyclyl containing one or two heteroatoms each independently selected from O, S, and N; wherein said S-atom might be substituted to form S(═O) or S(═O)₂;

Het³ represents a C-linked 5- or 6-membered monocyclic aromatic ring containing one, two or three heteroatoms each independently selected from O, S, and N;

Y represents O or CH₂;

X¹ represents CR⁶;

X² represents CR⁷;

X³ represents CR⁸;

R⁶, R⁷ and R⁸ each independently represent hydrogen, fluoro or chloro;

X⁴ represents O or NR⁵;

and the pharmaceutically acceptable salts and the solvates thereof.

The present invention also relates to a pharmaceutical composition comprising a therapeutically effective amount of a compound of Formula (I), a pharmaceutically acceptable salt, or a solvate thereof, and a pharmaceutically acceptable carrier or excipient.

Additionally, the invention relates to a compound of Formula (I), a pharmaceutically acceptable salt, or a solvate thereof, for use as a medicament, and to a compound of Formula (I), a pharmaceutically acceptable salt, or a solvate thereof, for use in the treatment or in the prevention of cancer.

In a particular embodiment, the invention relates to a compound of Formula (I), a pharmaceutically acceptable salt, or a solvate thereof, for use in the treatment or in the prevention of cancer.

The invention also relates to the use of a compound of Formula (I), a pharmaceutically acceptable salt, or a solvate thereof, in combination with an additional pharmaceutical agent for use in the treatment or prevention of cancer.

Furthermore, the invention relates to a process for preparing a pharmaceutical composition according to the invention, characterized in that a pharmaceutically acceptable carrier is intimately mixed with a therapeutically effective amount of a compound of Formula (I), a pharmaceutically acceptable salt, or a solvate thereof.

The invention also relates to a product comprising a compound of Formula (I), a pharmaceutically acceptable salt, or a solvate thereof, and an additional pharmaceutical agent, as a combined preparation for simultaneous, separate or sequential use in the treatment or prevention of cancer.

Additionally, the invention relates to a method of treating or preventing a cell proliferative disease in a subject which comprises administering to the said subject an effective amount of a compound of Formula (I), a pharmaceutically acceptable salt, or a solvate thereof, as defined herein, or a pharmaceutical composition or combination as defined herein.

### DETAILED DESCRIPTION OF THE INVENTION

The term ‘halo’ or ‘halogen’ as used herein represents fluoro, chloro, bromo and iodo.

The prefix ‘C_{x-y}’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain fully saturated hydrocarbon radical having from 1 to 4 carbon atoms, such as methyl, ethyl, n-propyl, isopropyl, n-butyl, s-butyl, t-butyl and the like.

The term ‘C₁₋₆alkyl’ as used herein as a group or part of a group represents a straight or branched chain fully saturated hydrocarbon radical having from 1 to 6 carbon atoms, such as methyl, ethyl, n-propyl, isopropyl, n-butyl, s-butyl, t-butyl, n-pentyl, n-hexyl and the like.

The term ‘C₂₋₇alkyl’ as used herein as a group or part of a group represents a straight or branched chain fully saturated hydrocarbon radical having from 2 to 7 carbon atoms, such as ethyl, n-propyl, isopropyl, n-butyl, s-butyl, t-butyl, n-pentyl, n-hexyl and the like.

The term ‘C₃₋₇cycloalkyl’ as used herein as a group or part of a group defines a fully saturated, cyclic hydrocarbon radical having from 3 to 7 carbon atoms, such as cyclopropyl, cyclobutyl, cyclopentyl and cyclohexyl.

The term “C₂₋₆alkenyl” as used herein as a group or part of a group represents a double bond containing straight or branched chain hydrocarbon radical having from 2 to 6 carbon atoms, such as ethenyl, propenyl, isopropenyl, buten-1-yl, (2Z)-buten-2-yl, (2E)-buten-2-yl, buten-3-yl, 2-methylpropen-1-yl, 1,3-butadiene, penten-1-yl, (2Z)-penten-2-yl, (2E)-penten-2-yl, (3Z)-penten-3-yl, (3E)-penten-3-yl, (4Z)-penten-4-yl, (4E)-penten-4-yl, penten-5-yl and the like.

The term “C₃₋₇cycloalkenyl” as used herein as a group or part of a group defines a double bond containing cyclic hydrocarbon radical having from 3 to 7 carbon atoms, such as cyclopropenyl, cyclobutenyl, cyclopentenyl, cyclohexenyl and cycloheptenyl.

The term “C₂₋₆alkynyl” as used herein as a group or part of a group represents a triple bond containing straight or branched chain hydrocarbon radical having from 2 to 6 carbon atoms, such as ethynyl, 1-propynyl, 2-propynyl, butyn-1-yl, butyn-2-yl, butyn-3-yl, 1,3-butadiyne, pentyn-1-yl, pentyn-2-yl, pentyn-3-yl, pentyn-5-yl and the like.

It will be clear for the skilled person that S(═O)₂ or SO₂ represents a sulfonyl moiety.

It will be clear for the skilled person that CO or C(═O) represents a carbonyl moiety.

Non-limiting examples of two R groups taken together to form together with the N-atom to which they are attached a 4- to 7-membered monocyclic fully saturated heterocyclyl containing one N-atom and optionally one additional heteroatom selected from O, S, and N, include, but are not limited to N-linked azetidinyl, N-linked pyrrolidinyl, N-linked morpholinyl, N-linked piperazinyl, and N-linked piperidinyl.

Non-limiting examples of 4- to 7-membered monocyclic fully saturated heterocyclyl containing one or two heteroatoms each independently selected from O, S, and N, include, but are not limited to tetrahydropyranyl, piperazinyl, tetrahydrofuranyl, 1,4-dioxanyl, tetrahydropyranyl, 1,4-oxazepanyl, 1,3-dioxolanyl, morpholinyl and azetidinyl.

Non-limiting examples of C-linked 4- to 7-membered monocyclic fully saturated heterocyclyl containing one or two heteroatoms each independently selected from O, S, and N, include, but are not limited to C-linked tetrahydropyranyl, C-linked piperazinyl, C-linked tetrahydrofuranyl, C-linked 1,4-dioxanyl, C-linked tetrahydropyranyl, C-linked 1,4-oxazepanyl, C-linked 1,3-dioxolanyl, C-linked morpholinyl and C-linked azetidinyl.

Within the context of this invention, bicyclic 6- to 11-membered bicyclic fully saturated heterocyclyl groups, or 6- to 11-membered bicyclic fully saturated ring systems, include fused, spiro and bridged bicycles.

Fused bicyclic groups are two cycles that share two atoms and the bond between these atoms.

Spiro bicyclic groups are two cycles that are joined at a single atom.

Bridged bicyclic groups are two cycles that share more than two atoms.

Examples of 6- to 11-membered bicyclic fully saturated ring systems optionally containing one or two heteroatoms each independently selected from O, S, and N, include, but are not limited to

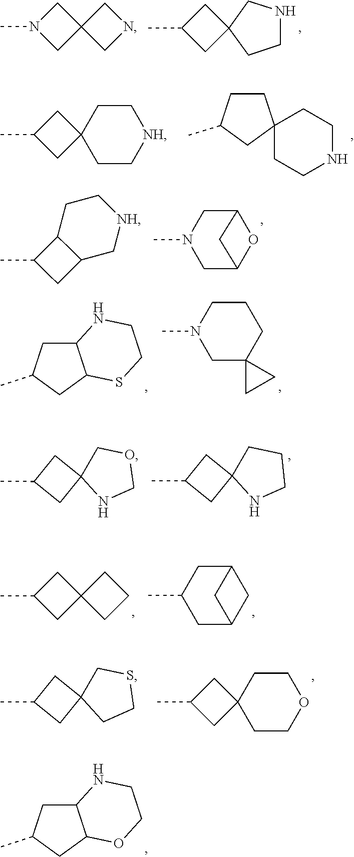

and the like.

Examples of two R groups taken together to form together with the N-atom to which they are attached a 6- to 11-membered bicyclic fully saturated heterocyclyl containing one N-atom and optionally one or two additional heteroatoms each independently selected from O, S, and N, include, but are not limited to

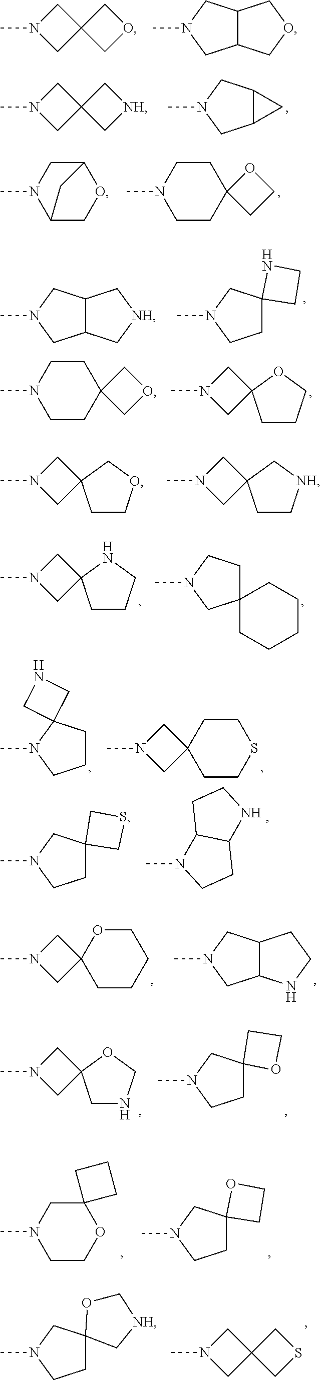

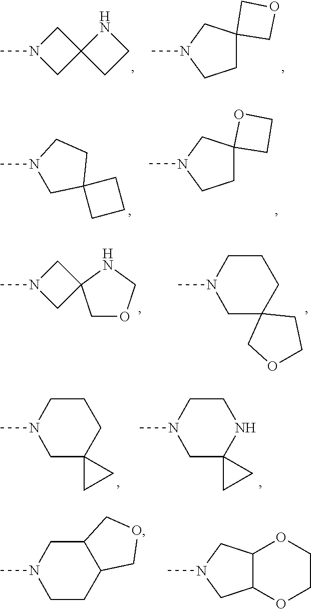

and the like.

Non-limiting examples of 5- to 6-membered monocyclic aromatic ring containing one or two heteroatoms each independently selected from O, S, and N, include, but are not limited to

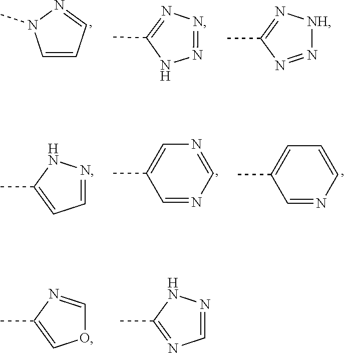

and the like.

Non-limiting examples of C-linked 5-or 6-membered monocyclic aromatic ring containing one or two heteroatoms each independently selected from O, S, and N, include, but are not limited to

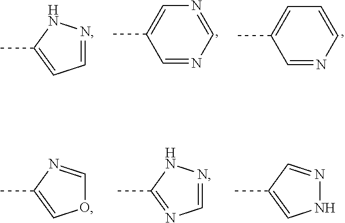

and the like.

Unless otherwise specified or clear from the context, heterocyclyl groups (e.g. Het¹), aromatic rings containing an heteroatom (e.g. Het²), or ring systems containing an heteroatom (e.g. Cy¹), can be attached to the remainder of the molecule of Formula (I) through any available ring carbon atom (C-linked) or nitrogen atom (N-linked) if available.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e. a compound that is sufficiently robust to survive isolation to a useful degree of purity from a reaction mixture.

Combinations of substituents and/or variables are permissible only if such combinations result in chemically stable compounds. ‘Stable compound’ is meant to indicate a compound that is sufficiently robust to survive isolation to a useful degree of purity from a reaction mixture.

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

When any variable occurs more than one time in any constituent, each definition is independent.

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, or subject (e.g., human) that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

The term “compound(s) of the (present) invention” or “compound(s) according to the (present) invention” as used herein, is meant to include the compounds of Formula (I) and the pharmaceutically acceptable salts, and the solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers. Where the stereochemistry of any particular chiral atom is not specified in the structures shown herein, then all stereoisomers are contemplated and included as the compounds of the invention, either as a pure stereoisomer or as a mixture of two or more stereoisomers.

Hereinbefore and hereinafter, the term “compound of Formula (I)” is meant to include the stereoisomers thereof and the tautomeric forms thereof. However where stereochemistry, as mentioned in the previous paragraph, is specified by bonds which are shown as solid wedged or hashed wedged bonds, or are otherwise indicated as having a particular configuration (e.g. R, S), or when the stereochemistry around a double bond is shown (e.g. in Formula (I)), then that stereoisomer is so specified and defined. It will be clear this also applies to subgroups of Formula (I).

It follows that a single compound may, where possible, exist in both stereoisomeric and tautomeric form.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Diastereomers (or diastereoisomers) are stereoisomers that are not enantiomers, i.e. they are not related as mirror images. If a compound contains a double bond, the substituents may be in the E or the Z configuration.

Substituents on bivalent cyclic saturated or partially saturated radicals may have either the cis- or trans-configuration; for example if a compound contains a disubstituted cycloalkyl group, the substituents may be in the cis or trans configuration.

Therefore, the invention includes enantiomers, diastereomers, racemates, E isomers, Z isomers, cis isomers, trans isomers and mixtures thereof, unless the context indicates otherwise and whenever chemically possible.

The meaning of all those terms, i.e. enantiomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer.

Pharmaceutically acceptable salts, in particular pharmaceutically acceptable additions salts, include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I), and solvates thereof, are able to form.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term solvate comprises the solvent addition forms as well as the salts thereof, which the compounds of Formula (I) are able to form. Examples of such solvent addition forms are e.g. hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, N-oxides and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C ¹³N, ¹⁵O¹⁷O¹⁸O³²P, ³³P, ³⁵S, ¹⁸F, ³⁶Cl, ¹²²I, ¹²³I, ¹²⁵I, ¹³¹I, ⁷⁵Br, ⁷⁶Br, ⁷⁷Br and ⁸²Br. Preferably, the isotope is selected from the group of ²H, ³H, ¹¹C and ¹⁸F. More preferably, the isotope is ²H. In particular, deuterated compounds are intended to be included within the scope of the present invention.

Certain isotopically-labeled compounds of the present invention (e.g., those labeled with ³H and ¹⁴C) may be useful for example in substrate tissue distribution assays. Tritiated (3H) and carbon-14 (¹⁴C) isotopes are useful for their ease of preparation and detectability.

Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies. PET imaging in cancer finds utility in helping locate and identify tumours, stage the disease and determine suitable treatment. Human cancer cells overexpress many receptors or proteins that are potential disease-specific molecular targets. Radiolabelled tracers that bind with high affinity and specificity to such receptors or proteins on tumour cells have great potential for diagnostic imaging and targeted radionuclide therapy (Charron, Carlie L. et al. Tetrahedron Lett. 2016, 57(37), 4119-4127). Additionally, target-specific PET radiotracers may be used as biomarkers to examine and evaluate pathology, by for example, measuring target expression and treatment response (Austin R. et al. Cancer Letters (2016), doi: 10.1016/j.canlet.2016.05.008).

In an embodiment, the present invention concerns novel compounds of Formula (I), wherein

R^{1a} and R^{1b} are each independently selected from the group consisting of C₁₋₆alkyl, Het¹, and Ar, wherein said C₁₋₆alkyl is optionally substituted with one or two R²;

or R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said heterocyclyl is optionally substituted with one or two substituents each independently selected from the group consisting of OR^{f}, NR^{d}R^{e}, CN, halo, CF₃, and C₁₋₄alkyl optionally substituted with one substituent selected from the group consisting of OR and CN;

or R^{1a} and R^{1b} are taken together to form together with the N-atom to which they are attached a 6- to 11-membered bicyclic fully saturated heterocyclyl containing one N-atom and optionally one or two additional heteroatoms each independently selected from O, S, and N, wherein said heterocyclyl is optionally substituted with one or two substituents each independently selected from the group consisting of halo and C₁₋₄alkyl; each R² is independently selected from the group consisting of OR^{f}, CF₃, NR^{m}Rⁿ, SO₂R, Het¹, and Het²,

R represents C₁₋₆alkyl;

R^{m} and Rⁿ are each independently selected from the group consisting of C₂₋₇alkyl optionally substituted with one or two O^{R} substituents;

R^{d} and R^{e} are taken together to form together with the N-atom to which they are attached a 4- to 7-membered monocyclic fully saturated heterocyclyl containing one N-atom and optionally one additional heteroatom selected from O, S, and N;

n is 1 or 2;

R^{f} is selected from the group consisting of hydrogen, C₁₋₆alkyl, Het¹, Het², wherein said C₁₋₆alkyl is optionally substituted with one O^{R} substituent;

Het¹ represents a 4- to 7-membered monocyclic fully saturated heterocyclyl containing one or two heteroatoms each independently selected from O, S, and N, wherein said heterocyclyl is optionally substituted with one or two substituents each independently selected from the group consisting of halo, CF₃, and C₁₋₄alkyl optionally substituted with one ORⁱ substituent;

Het² represents a 5- to 6-membered monocyclic aromatic ring containing one, two, three or four heteroatoms each independently selected from O, S, and N, wherein said aromatic ring is optionally substituted with one or two substituents each independently selected from the group consisting of halo and C₁₋₄alkyl;

Ar¹ represents phenyl;

Rⁱ represents C₁₋₆alkyl;

R³ represents hydrogen, C₁₋₄alkyl-OH;

R⁴ represents hydrogen or methyl;

R⁵ represents —(C═O)-Het³; wherein said Het³ is optionally substituted with one or two substituents selected from methyl or methoxy;

Het³ represents a C-linked 5-or 6-membered monocyclic aromatic ring containing one, two or three heteroatoms each independently selected from O, S, and N;

Y represents O or CH₂;

X¹ represents CR⁶;

X² represents CR⁷;

X³ represents CR⁸;

R⁶, R⁷ and R⁸ each independently represent hydrogen or fluoro;

X⁴ represents O or NR⁵;

and the pharmaceutically acceptable salts and the solvates thereof.

In an embodiment, the present invention concerns novel compounds of Formula (I), wherein

R^{1a} and R^{1b} are each independently selected from the group consisting of hydrogen, C₁₋₆alkyl, C₂₋₆alkenyl, C₂₋₆alkynyl, C₃₋₇cycloalkyl, C₃₋₇cycloalkenyl, Het¹, Ar¹, Het², and Cy¹, wherein said C₁₋₆alkyl, C₂₋₆alkenyl, C₂₋₆alkynyl, C₃₋₇cycloalkyl or C₃₋₇cycloalkenyl is optionally substituted with one or two R²;

or R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of oxo, ORⁱ, SRⁱ, NR^{d}R^{e}, CN, halo, CF₃, and C₁₋₄alkyl optionally substituted with one substituent selected from the group consisting of ORⁱ, SRⁱ, CN and halo;

or R^{1a} and R^{1b} are taken together to form together with the N-atom to which they are attached a 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of oxo, ORⁱ, SRⁱ, NR^{d}R^{e}, CN, halo, CF₃, and C₁₋₄alkyl optionally substituted with one substituent selected from the group consisting of ORⁱ, SRⁱ, CN and halo;

each R² is independently selected from the group consisting of OR^{f}, SR^{f}, CN, halo, CF₃, NR^{m}Rⁿ, SO₂R^{c}, C(═O)R^{c}, C(═O)OR^{d}, C(═O)NR^{d}R^{e}, SO₂NR^{d}R^{e}, C₃₋₇cycloalkyl, C₃₋₇ cycloalkenyl, Het¹, Ar¹, Het², and Cy¹, wherein said C₃₋₇cycloalkyl or C₃₋₇cycloalkenyl is optionally substituted with one or two substituents each independently selected from the group consisting of OR^{f}, SR^{f}, CN, halo and NR^{d}R^{e};

R^{c} is selected from the group consisting of C₁₋₆alkyl, C₃₋₇cycloalkyl, Het¹, Ar¹ and Het²;

R^{m} and Rⁿ are each independently selected from the group consisting of hydrogen, methyl, C₂₋₇-alkyl, C₃₋₇cycloalkyl, Het¹, Ar¹, and Het², wherein said C₂₋₇-alkyl or C₃₋₇cycloalkyl is optionally substituted with one or two substituents each independently selected from the group consisting of ORⁱ, SRⁱ, NR^{g}R^{h}, CN, halo, and C₁₋₄alkyl optionally substituted with one substituent selected from the group consisting of ORⁱ, SRⁱ, CN, NR^{g}R^{h} and halo;

R^{d} and R^{e} are each independently selected from the group consisting of hydrogen, methyl, C₂₋₇alkyl, C₃₋₇cycloalkyl, Het¹, Ar¹, and Het², wherein said C₂₋₇alkyl or C₃₋₇cycloalkyl is optionally substituted with one or two substituents each independently selected from the group consisting of ORⁱ, SRⁱ, NR^{g}R^{h}, CN, halo, and C₁₋₄alkyl optionally substituted with one substituent selected from the group consisting of ORⁱ, SRⁱ, CN, NR^{g}R^{h} and halo;

or R^{d} and R^{e} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo; or R^{d} and R^{e} are taken together to form together with the N-atom to which they are attached a fused 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;

n is 1 or 2;

R^{f} is selected from the group consisting of hydrogen, C₁₋₆alkyl, CF₃, C₃₋₇cycloalkyl, Het¹, Ar¹, Het², wherein said C₁₋₆alkyl or C₃₋₇cycloalkyl is optionally substituted with one substituent selected from the group consisting of ORⁱ, SRⁱ, CN, halo, NR^{m}Rⁿ, SO₂R, C(═O)R^{o}, C(═O)OR^{d}, C(═O)NR^{d}R^{e}, SO₂NR^{d}R^{e}, C₃₋₇cycloalkyl, Het¹, Ar¹ and Het²; R^{g} and R^{h} are each independently selected from the group consisting of hydrogen, C₁₋₆alkyl and C₃₋₇cycloalkyl;

or R^{g} and R^{h} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂;

Het¹ represents a 4- to 7-membered monocyclic fully saturated heterocyclyl containing one or two heteroatoms each independently selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;

Het² represents a 5- to 6-membered monocyclic aromatic ring containing one or two heteroatoms each independently selected from O, S, and N, wherein said S-atom might be substituted to form S(═O) or S(═O)₂, and wherein said aromatic ring is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;

Cy¹ represents a 6- to 11-membered bicyclic fully saturated ring system optionally containing one or two heteroatoms each independently selected from O, S, and N, wherein said S-atom might be substituted to form S(═O) or S(═O)₂, and wherein said ring system is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of OR, SRⁱ, CN and halo;

Ar¹ represents phenyl optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;

Rⁱ represents hydrogen, C₁₋₆alkyl or C₃₋₇cycloalkyl;

R^{j} and R^{k} are each independently selected from the group consisting of hydrogen, C₁₋₆alkyl and C₃₋₇cycloalkyl;

Y represents O or CH₂;

X¹ represents CH;

X² represents CH;

X³ represents CH;

R³ represents hydrogen;

R⁴ represents methyl;

X⁴ represents O;

and the pharmaceutically acceptable salts and the solvates thereof.

In an embodiment, the present invention concerns novel compounds of Formula (I), wherein

R^{1a} and R^{1b} are each independently selected from the group consisting of hydrogen, C₁₋₆alkyl, C₂₋₆alkenyl, C₂₋₆alkynyl, C₃₋₇cycloalkyl, C₃₋₇cycloalkenyl, Het¹, Ar¹, Het², and Cy¹, wherein said C₁₋₆alkyl, C₂₋₆alkenyl, C₂₋₆alkynyl, C₃₋₇cycloalkyl or C₃₋₇cycloalkenyl is optionally substituted with one or two R²;

or R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of oxo, ORⁱ, SRⁱ, CN, halo, CF₃, and C₁₋₄alkyl optionally substituted with one substituent selected from the group consisting of ORⁱ, SRⁱ, CN and halo; or R^{1a} and R^{1b} are taken together to form together with the N-atom to which they are attached a 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of oxo, ORⁱ, SRⁱ, CN, halo, CF₃, and C₁₋₄alkyl optionally substituted with one substituent selected from the group consisting of ORⁱ, SRⁱ, CN and halo;

each R² is independently selected from the group consisting of ORⁱ, SRⁱ, CN, halo, CF₃, NR^{m}Rⁿ, SO₂R^{o}, C(═O)R^{o}, C₃₋₇cycloalkyl, C₃₋₇cycloalkenyl, Het¹, Ar¹, Het², and Cy¹, wherein said C₃₋₇cycloalkyl or C₃₋₇cycloalkenyl is optionally substituted with one or two substituents each independently selected from the group consisting of OR^{f}, SR^{f}, CN, and halo;

R^{c} is selected from the group consisting of C₁₋₆alkyl, C₃₋₇cycloalkyl, Het¹, Ar¹ and Het²;

R^{m} and Rⁿ are each independently selected from the group consisting of hydrogen, methyl, C₂₋₇-alkyl, C₃₋₇cycloalkyl, Het¹, Ar¹, and Het², wherein said C₂₋₇-alkyl or C₃₋₇cycloalkyl is optionally substituted with one or two substituents each independently selected from the group consisting of ORⁱ, SRⁱ, NR^{g}R^{h}, CN, halo, and C₁₋₄alkyl optionally substituted with one substituent selected from the group consisting of ORⁱ, SRⁱ, CN, NR^{g}R^{h} and halo;

n is 1 or 2;

R^{f} is selected from the group consisting of hydrogen, C₁₋₆alkyl, CF₃, C₃₋₇cycloalkyl, Het¹, Ar¹, Het², wherein said C₁₋₆alkyl or C₃₋₇cycloalkyl is optionally substituted with one substituent selected from the group consisting of ORⁱ, SRⁱ, CN, halo, NR^{m}Rⁿ, SO₂R^{o}, C(═O)R^{o}, C₃₋₇cycloalkyl, Het¹, Ar¹ and Het²;

R^{g} and R^{h} are each independently selected from the group consisting of hydrogen, C₁₋₆ alkyl and C₃₋₇cycloalkyl;

or R^{g} and R^{h} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂;

Het¹ represents a 4- to 7-membered monocyclic fully saturated heterocyclyl containing one or two heteroatoms each independently selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;

Het² represents a 5- to 6-membered monocyclic aromatic ring containing one or two heteroatoms each independently selected from O, S, and N, wherein said S-atom might be substituted to form S(═O) or S(═O)₂, and wherein said aromatic ring is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;

Cy¹ represents a 6- to 11-membered bicyclic fully saturated ring system optionally containing one or two heteroatoms each independently selected from O, S, and N, wherein said S-atom might be substituted to form S(═O) or S(═O)₂, and wherein said ring system is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;

Ar¹ represents phenyl optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;

Rⁱ represents hydrogen, C₁₋₆alkyl or C₃₋₇cycloalkyl;

R^{j} and R^{k} are each independently selected from the group consisting of hydrogen, C₁₋₆alkyl and C₃₋₇cycloalkyl;

Y represents O or CH₂;

X¹ represents CH;

X² represents CH;

X³ represents CH;

R³ represents hydrogen;

R⁴ represents methyl;

X⁴ represents O;

and the pharmaceutically acceptable salts and the solvates thereof.

In an embodiment, the present invention concerns novel compounds of Formula (I), wherein

R^{1a} and R^{1b} are each independently selected from the group consisting of hydrogen, C₁₋₆alkyl, C₂₋₆alkenyl, C₂₋₆alkynyl, C₃₋₇cycloalkyl, C₃₋₇cycloalkenyl, Het¹, Ar¹, Het², and Cy¹,

wherein said C₁₋₆alkyl, C₂₋₆alkenyl, C₂₋₆alkynyl, C₃₋₇cycloalkyl or C₃₋₇cycloalkenyl is optionally substituted with one or two R²;

each R² is independently selected from the group consisting of OR^{f}, SR^{f}, CN, halo, CF₃, NR^{m}Rⁿ, SO₂R^{o}, C(═O)R^{o}, C₃₋₇cycloalkyl, C₃₋₇cycloalkenyl, Het¹, Ar¹, Het², and Cy¹, wherein said C₃₋₇cycloalkyl or C₃₋₇cycloalkenyl is optionally substituted with one or two substituents each independently selected from the group consisting of OR^{f}, SR^{f}, CN, and halo;

R^{c} is selected from the group consisting of C₁₋₆alkyl, C₃₋₇cycloalkyl, Het¹, Ar¹ and Het²;

R^{m} and Rⁿ are each independently selected from the group consisting of hydrogen, methyl, C₂₋₇-alkyl, C₃₋₇cycloalkyl, Het¹, Ar¹, and Het², wherein said C₂₋₇-alkyl or C₃₋₇cycloalkyl is optionally substituted with one or two substituents each independently selected from the group consisting of ORⁱ, SRⁱ, NR^{g}R^{h}, CN, halo, and C₁₋₄alkyl optionally substituted with one substituent selected from the group consisting of ORⁱ, SRⁱ, CN, NR^{g}R^{h} and halo;

n is 1 or 2;

R^{f} is selected from the group consisting of hydrogen, C₁₋₆alkyl, CF₃, C₃₋₇cycloalkyl, Het¹, Ar¹, Het², wherein said C₁₋₆alkyl or C₃₋₇cycloalkyl is optionally substituted substituted with one substituent selected from the group consisting of ORⁱ, SRⁱ, CN, halo, NR^{m}Rⁿ, SO₂R^{o}, C(═O)R^{o}, C₃₋₇cycloalkyl, Het¹, Ar¹ and Het²;

R^{g} and R^{h} are each independently selected from the group consisting of hydrogen, C₁₋₆ alkyl and C₃₋₇cycloalkyl;

or R^{g} and R^{h} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂;

Het¹ represents a 4- to 7-membered monocyclic fully saturated heterocyclyl containing one or two heteroatoms each independently selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;

Het² represents a 5- to 6-membered monocyclic aromatic ring containing one or two heteroatoms each independently selected from O, S, and N, wherein said S-atom might be substituted to form S(═O) or S(═O)₂, and wherein said aromatic ring is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;

Cy¹ represents a 6- to 11-membered bicyclic fully saturated ring system optionally containing one or two heteroatoms each independently selected from O, S, and N, wherein said S-atom might be substituted to form S(═O) or S(═O)₂, and wherein said ring system is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;

Ar¹ represents phenyl optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;

Rⁱ represents hydrogen, C₁₋₆alkyl or C₃₋₇cycloalkyl;

R^{j} and R^{k} are each independently selected from the group consisting of hydrogen, C₁₋₆alkyl and C₃₋₇cycloalkyl;

Y represents O or CH₂;

X¹ represents CH;

X² represents CH;

X³ represents CH;

R³ represents hydrogen;

R⁴ represents methyl;

X⁴ represents O;

and the pharmaceutically acceptable salts and the solvates thereof.

In an embodiment, the present invention concerns novel compounds of Formula (I), wherein

R^{1a} and R^{1b} are each independently selected from the group consisting of

C₁₋₆alkyl, Ar¹, and Cy¹,

wherein said C₁₋₆alkyl is optionally substituted with one R²

or R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of ORE, CF₃, and C₁₋₄alkyl optionally substituted with one OR^{f};

or R^{1a} and R^{1b} are taken together to form together with the N-atom to which they are attached a 6- to 11-membered bicyclic fully saturated heterocyclyl containing one N-atom and optionally one or two additional heteroatoms each independently selected from O, S, and N, wherein said S-atom might be substituted to form S(═O) or S(═O)₂;

R² is selected from the group consisting of OR, CF₃, Het¹, and Het²;

n is 1 or 2;

R^{f} is selected from the group consisting of hydrogen, C₁₋₆alkyl, Het¹, and C₁₋₆alkyl substituted with one OW;

Het¹ represents a 4- to 7-membered monocyclic fully saturated heterocyclyl containing one or two heteroatoms each independently selected from O, S, and N, wherein said S-atom might be substituted to form S(═O) or S(═O)₂, and wherein said heterocyclyl is optionally substituted with one or two halo;

Het² represents a 5- to 6-membered monocyclic aromatic ring containing one or two heteroatoms each independently selected from O, S, and N, wherein said S-atom might be substituted to form S(═O) or S(═O)₂, and wherein said aromatic ring is optionally substituted with one or two C₁₋₄alkyl;

Cy¹ represents a 6- to 11-membered bicyclic fully saturated ring system optionally containing one or two heteroatoms each independently selected from O, S, and N, wherein said S-atom might be substituted to form S(═O) or S(═O)₂, and wherein said ring system is optionally substituted with one or two halo;

Ar¹ represents phenyl;

Rⁱ represents C₁₋₆alkyl;

Y represents CH₂;

X¹ represents CH;

X² represents CH;

X³ represents CH;

R³ represents hydrogen;

R⁴ represents methyl;

X⁴ represents O;

and the pharmaceutically acceptable salts and the solvates thereof.

In an embodiment, the present invention concerns novel compounds of Formula (I), wherein

R^{1a} and R^{1b} are each independently selected from the group consisting of C₁₋₆alkyl optionally substituted with one R²

R² is selected from the group consisting of OR and Het¹;

n is 2;

R^{f} is selected from the group consisting of hydrogen, C₁₋₆alkyl, Het¹, and C₁₋₆alkyl substituted with one ORⁱ;

Het¹ represents a 4- to 7-membered monocyclic fully saturated heterocyclyl containing one or two heteroatoms each independently selected from O, S, and N, wherein said S-atom might be substituted to form S(═O) or S(═O)₂, and wherein said heterocyclyl is optionally substituted with one or two halo;

Ar¹ represents phenyl;

Rⁱ represents C₁₋₆alkyl;

Y represents CH₂;

X¹ represents CH;

X² represents CH;

X³ represents CH;

R³ represents hydrogen;

R⁴ represents methyl;

X⁴ represents O

and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein R^{1a} represents methyl.

In an embodiment, the present invention relates to those compounds of Formula (I) and the pharmaceutically acceptable salts, and the solvates thereof, or any subgroup thereof as mentioned in any of the other embodiments, wherein

Y represents O.

In an embodiment, the present invention relates to those compounds of Formula (I) and the pharmaceutically acceptable salts, and the solvates thereof, or any subgroup thereof as mentioned in any of the other embodiments, wherein

Y represents CH₂.

In an embodiment, the present invention relates to those compounds of Formula (I) and the pharmaceutically acceptable salts, and the solvates thereof, or any subgroup thereof as mentioned in any of the other embodiments, wherein n is 1.

In an embodiment, the present invention relates to those compounds of Formula (I) and the pharmaceutically acceptable salts, and the solvates thereof, or any subgroup thereof as mentioned in any of the other embodiments, wherein n is 2.

In an embodiment, the present invention relates to those compounds of Formula (I) and the pharmaceutically acceptable salts, and the solvates thereof, or any subgroup thereof as mentioned in any of the other embodiments, wherein R^{1a} is not taken together with R^{1b} to form a heterocyclyl.

In an embodiment, the present invention relates to those compounds of Formula (I) and the pharmaceutically acceptable salts, and the solvates thereof, or any subgroup thereof as mentioned in any of the other embodiments, wherein R^{d} is not taken together with R^{e} to form a heterocyclyl.

In an embodiment, the present invention relates to those compounds of Formula (I) and the pharmaceutically acceptable salts, and the solvates thereof, or any subgroup thereof as mentioned in any of the other embodiments, wherein R^{1a} is taken together with R^{1b}.

In an embodiment, the present invention relates to those compounds of Formula (I) and the pharmaceutically acceptable salts, and the solvates thereof, or any subgroup thereof as mentioned in any of the other embodiments, wherein R^{1a} and R^{1b} are taken together to form together with the N-atom to which they are attached a heterocyclyl as defined in any one of the other embodiments.

In an embodiment, the present invention relates to those compounds of Formula (I) and the pharmaceutically acceptable salts, and the solvates thereof, or any subgroup thereof as mentioned in any of the other embodiments, wherein R^{1a} and R^{1b} are taken together to form together with the N-atom to which they are attached a monocyclic heterocyclyl as defined in any one of the other embodiments.

In an embodiment, the present invention relates to those compounds of Formula (I) and the pharmaceutically acceptable salts, and the solvates thereof, or any subgroup thereof as mentioned in any of the other embodiments, wherein R^{1a} and Rib are taken together to form together with the N-atom to which they are attached a bicyclic heterocyclyl as defined in any one of the other embodiments.

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are each independently selected from the group consisting of

hydrogen, C₁₋₆alkyl, C₂₋₆alkenyl, C₂₋₆alkynyl, C₃₋₇cycloalkyl, C₃₋₇cycloalkenyl, Het¹, Ar¹, Het², and Cy¹,

wherein said C₁₋₆alkyl, C₂₋₆alkenyl, C₂₋₆alkynyl, C₃₋₇cycloalkyl or C₃₋₇cycloalkenyl is optionally substituted with one or two R².

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are each independently selected from the group consisting of

C₁₋₆alkyl, C₂₋₆alkenyl, C₂₋₆alkynyl, C₃₋₇cycloalkyl, C₃₋₇cycloalkenyl, Het¹, Ar¹, Het², and Cy¹,

wherein said C₁₋₆alkyl, C₂₋₆alkenyl, C₂₋₆alkynyl, C₃₋₇cycloalkyl or C₃₋₇cycloalkenyl is optionally substituted with one or two R².

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are each independently selected from the group consisting of

C₁₋₆alkyl, C₂₋₆alkenyl, C₂₋₆alkynyl, C₃₋₇cycloalkyl, and C₃₋₇cycloalkenyl,

wherein said C₁₋₆alkyl, C₂₋₆alkenyl, C₂₋₆alkynyl, C₃₋₇cycloalkyl or C₃₋₇cycloalkenyl is optionally substituted with one or two R².

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are each independently selected from the group consisting of

C₁₋₆alkyl optionally substituted with one R².

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are each independently selected from the group consisting of

Het¹, Ar¹, and C₁₋₆alkyl optionally substituted with one R².

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are each independently selected from the group consisting of

Het¹, Ar¹, and C₁₋₆alkyl optionally substituted with one R², and

R² is selected from the group consisting of ORE, CF₃, NR^{m}Rⁿ, SO₂R^{o}, Het¹, and Het².

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are each independently selected from the group consisting of

C₁₋₆alkyl optionally substituted with one R²; and

R² is selected from the group consisting of OR and Het¹.

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are each independently selected from the group consisting of

C₁₋₆alkyl optionally substituted with one R²; and

R² is selected from the group consisting of OR and Het¹; and

n is 2.

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are not hydrogen.

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of oxo, ORE, SR, NR^{d}R^{e}, CN, halo, CF₃, and C₁₋₄alkyl optionally substituted with one substituent selected from the group consisting of OR^{f}, SR^{f}, CN and halo.

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are taken together to form together with the N-atom to which they are attached a 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of oxo, ORⁱ, SRⁱ, NR^{d}R^{e}, CN, halo, CF₃, and C₁₋₄alkyl optionally substituted with one substituent selected from the group consisting of OR^{f}, SR^{f}, CN and halo.

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said heterocyclyl is optionally substituted with one or two substituents each independently selected from the group consisting of OR, NR^{d}R^{e}, CN, halo, CF₃, and C₁₋₄alkyl optionally substituted with one substituent selected from the group consisting of OR and CN.

In an embodiment, the present invention relates to those compounds of Formula (I) and the pharmaceutically acceptable salts, and the solvates thereof, or any subgroup thereof as mentioned in any of the other embodiments, wherein

R^{1a} and R^{1b} are taken together to form together with the N-atom to which they are attached a 6- to 11-membered bicyclic fully saturated heterocyclyl containing one N-atom and optionally one or two additional heteroatoms each independently selected from O, S, and N, wherein said heterocyclyl is optionally substituted with one or two substituents each independently selected from the group consisting of halo and C₁₋₄ alkyl.

In an embodiment, the present invention relates to those compounds of Formula (I) and the pharmaceutically acceptable salts, and the solvates thereof, or any subgroup thereof as mentioned in any of the other embodiments, wherein each R² is independently selected from the group consisting of ORE, CF₃, NR^{m}Rⁿ, SO₂R^{o}, Het¹, and Het².

In an embodiment, the present invention relates to those compounds of Formula (I) and the pharmaceutically acceptable salts, and the solvates thereof, or any subgroup thereof as mentioned in any of the other embodiments, wherein

R³ represents hydrogen;

R⁴ represents methyl;

X⁴ represents O.

In an embodiment, the present invention relates to those compounds of Formula (I) and the pharmaceutically acceptable salts, and the solvates thereof, or any subgroup thereof as mentioned in any of the other embodiments, wherein X⁴ represents O.

In an embodiment, the present invention relates to those compounds of Formula (I) and the pharmaceutically acceptable salts, and the solvates thereof, or any subgroup thereof as mentioned in any of the other embodiments, wherein X⁴ represents NR⁵.

In an embodiment, the present invention relates to those compounds of Formula (I) and the pharmaceutically acceptable salts, and the solvates thereof, or any subgroup thereof as mentioned in any of the other embodiments, wherein R⁶, R⁷ and R⁸ each independently represent hydrogen or fluoro.

In an embodiment, the present invention relates to those compounds of Formula (I) and the pharmaceutically acceptable salts, and the solvates thereof, or any subgroup thereof as mentioned in any of the other embodiments, wherein R⁶, R⁷ and R⁸ represent hydrogen.

In an embodiment, the present invention relates to those compounds of Formula (I) and the pharmaceutically acceptable salts, and the solvates thereof, or any subgroup thereof as mentioned in any of the other embodiments, wherein R⁶, R⁷ and R⁸ represent fluoro.

In an embodiment, the present invention relates to those compounds of Formula (I) and the pharmaceutically acceptable salts, and the solvates thereof, or any subgroup thereof as mentioned in any of the other embodiments, wherein Het¹ is attached to the remainder of the molecule of Formula (I) through any available ring carbon atom.

In an embodiment, the present invention relates to those compounds of Formula (I) and the pharmaceutically acceptable salts, and the solvates thereof, or any subgroup thereof as mentioned in any of the other embodiments, wherein Het¹ is attached to the remainder of the molecule of Formula (I) through any available ring nitrogen atom.

In an embodiment, the present invention relates to those compounds of Formula (I) and the pharmaceutically acceptable salts, and the solvates thereof, or any subgroup thereof as mentioned in any of the other embodiments, wherein Het¹ is

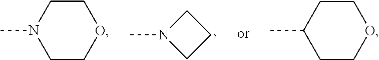

each optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein Het¹ is

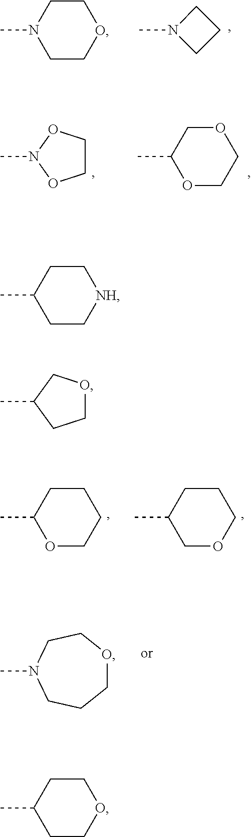

each optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein Cy¹ is

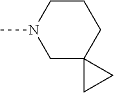

optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein Het² is

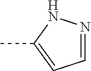

optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein Het² is

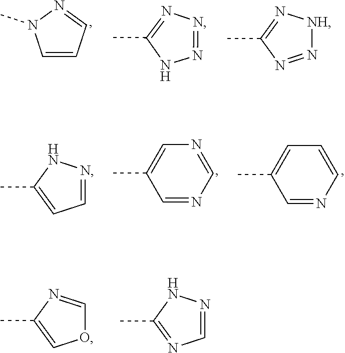

optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein Het³ is

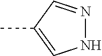

optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein Het² represents a 5- to 6-membered monocyclic aromatic ring containing one or two heteroatoms each independently selected from O, S, and N, and wherein said aromatic ring is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo.

In an embodiment, the present invention relates to those compounds of Formula (I) and the pharmaceutically acceptable salts, and the solvates thereof, or any subgroup thereof as mentioned in any of the other embodiments, wherein when R^{1a} and R^{1b} are taken together with the N-atom to which they are attached, together they form

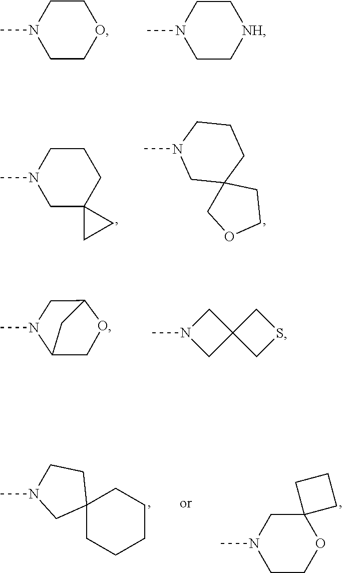

each optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein when R^{1a} and R^{1b} are taken together with the N-atom to which they are attached, together they form

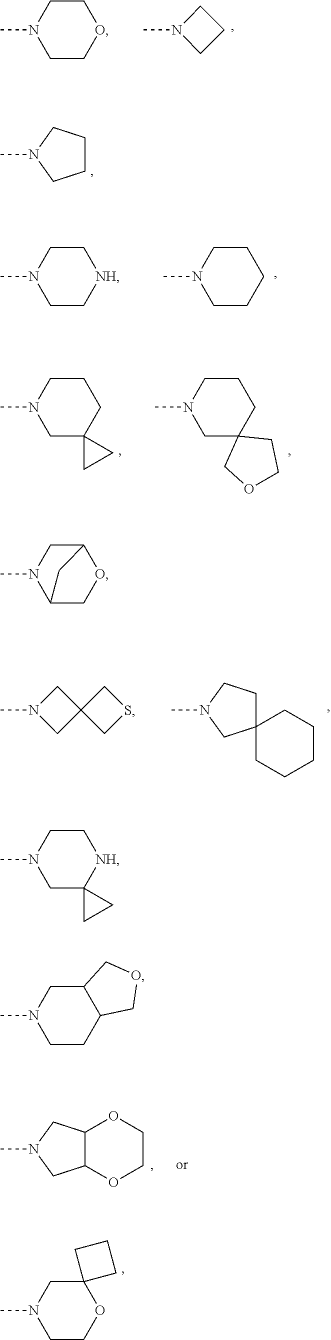

each optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein when R^{d} and R^{e} are taken together with the N-atom to which they are attached, together they form 1-morholinyl.

In an embodiment, the present invention relates to those compounds of Formula (I) and the pharmaceutically acceptable salts, and the solvates thereof, or any subgroup thereof as mentioned in any of the other embodiments, wherein Het² is attached to the remainder of the molecule of Formula (I) through any available ring carbon atom.

In an embodiment, the present invention relates to those compounds of Formula (I) and the pharmaceutically acceptable salts, and the solvates thereof, or any subgroup thereof as mentioned in any of the other embodiments, wherein Het² is attached to the remainder of the molecule of Formula (I) through any available ring nitrogen atom.

In an embodiment, the present invention relates to those compounds of Formula (I) and the pharmaceutically acceptable salts, and the solvates thereof, or any subgroup thereof as mentioned in any of the other embodiments, wherein Cy¹ is attached to the remainder of the molecule of Formula (I) through any available ring carbon atom.

In an embodiment, the present invention relates to those compounds of Formula (I) and the pharmaceutically acceptable salts, and the solvates thereof, or any subgroup thereof as mentioned in any of the other embodiments, wherein Cy¹ is attached to the remainder of the molecule of Formula (I) through any available ring nitrogen atom.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-1):

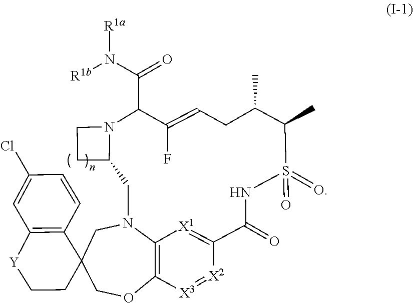

It will be clear that all variables in the structure of Formula (I-1), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-a′):

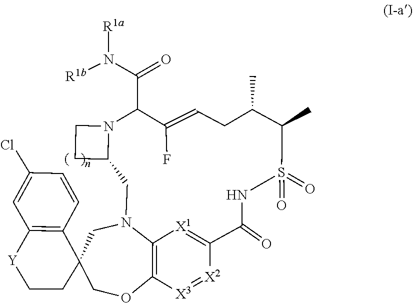

It will be clear that all variables in the structure of Formula (I-a′), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-a1):

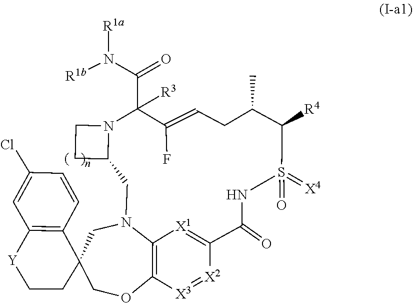

It will be clear that all variables in the structure of Formula (I-a1), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-b′):

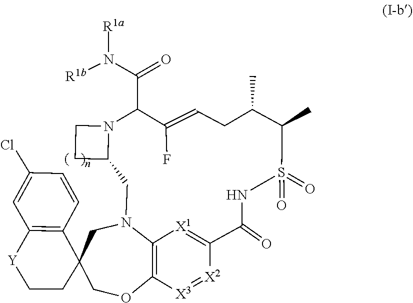

It will be clear that all variables in the structure of Formula (I-b′), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-b1):

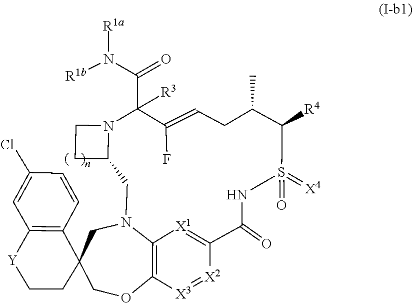

It will be clear that all variables in the structure of Formula (I-b1), are defined as defined for the compounds of Formula (I) or any subgroup thereof as mentioned in any of the other embodiments.

In an embodiment, the present invention relates to a subgroup of Formula (I) as defined in the general reaction schemes.

In an embodiment the compound of Formula (I) is selected from the group consisting of any of the exemplified compounds, and the free bases, the pharmaceutically acceptable salts, and the solvates thereof.

All possible combinations of the above indicated embodiments are considered to be embraced within the scope of the invention.

### Methods for the Preparation of Compounds

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or can be prepared by published methods. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art including also analogous reaction protocols as described in WO2016033486, WO2017147410 and WO2018183418.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

The meaning of the chemical abbreviations used in the schemes below are as defined in the schemes or as defined in Table 1.

The general schemes below focus on compounds of Formula (I-al) and subgroups thereof, but a skilled person will understand that compounds of Formula (I-b1) can be synthesized by using analogous reaction procedures:

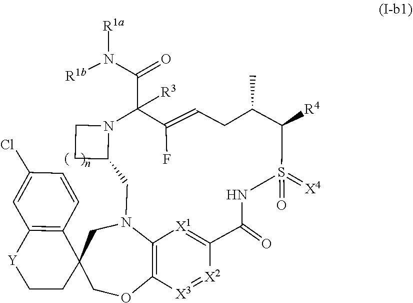

Compounds of Formula (I-a) wherein the variables are defined as in Formula (I) can be prepared according to Scheme 1,

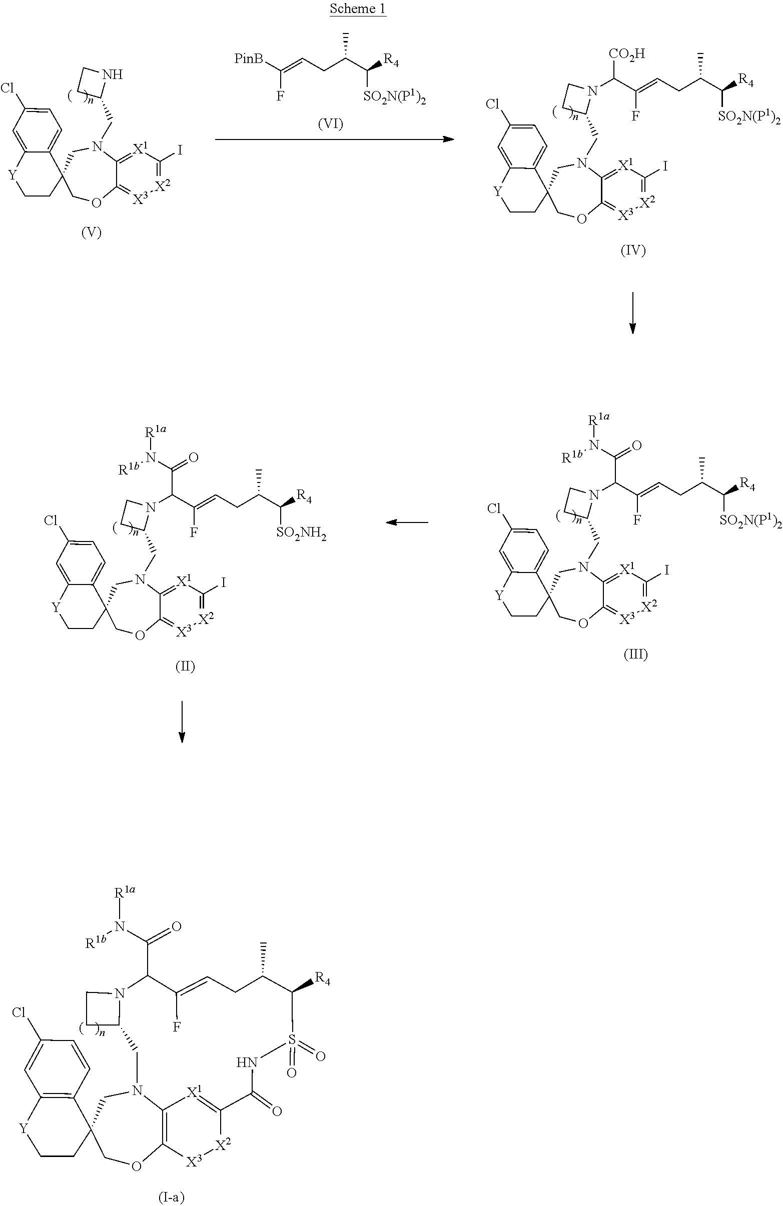

By reacting an intermediate of Formula (II) in the presence of a suitable palladium catalyst such as, for example, [1,1′-Bis(diphenylphosphino)ferrocene]palladium(II) dichloride, in a suitable solvent such as tetrahydrofuran, at a suitable temperature such as 80 or 100° C. and at a suitable CO pressure such as 30 bars.
Intermediates of Formula (II) can be prepared by deprotection of an intermediate of Formula (III) in which P¹ is a suitable protective group such as, for example, para-methoxybenzyl (PMB), with a suitable deprotecting agent such as, for example, trifluoroacetic acid, in a suitable solvent such as, for example, dichloromethane.
Intermediates of Formula (III) can be prepared by reacting an intermediate of Formula (IV) with an amine in presence of a suitable coupling agent such as, for example, propanephosphonic acid anhydride, a suitable base such as, for example, triethylamine, in a suitable solvent such as, for example, dichloromethane or ethyl acetate, at a suitable temperature such as, for instance, room temperature or 40° C.
Intermediates of Formula (IV) can be prepared by reacting an intermediate of Formula (V) with an intermediate of Formula (VI) (‘Pin’ means pinacol ester) and glyoxylic acid in a suitable solvent such as, for example, methanol, at a suitable temperature such as, for example, 40 or 60° C.

Alternatively compounds of Formula (I-a) wherein the variables are defined as in Formula (I) can be prepared according to Scheme 2,

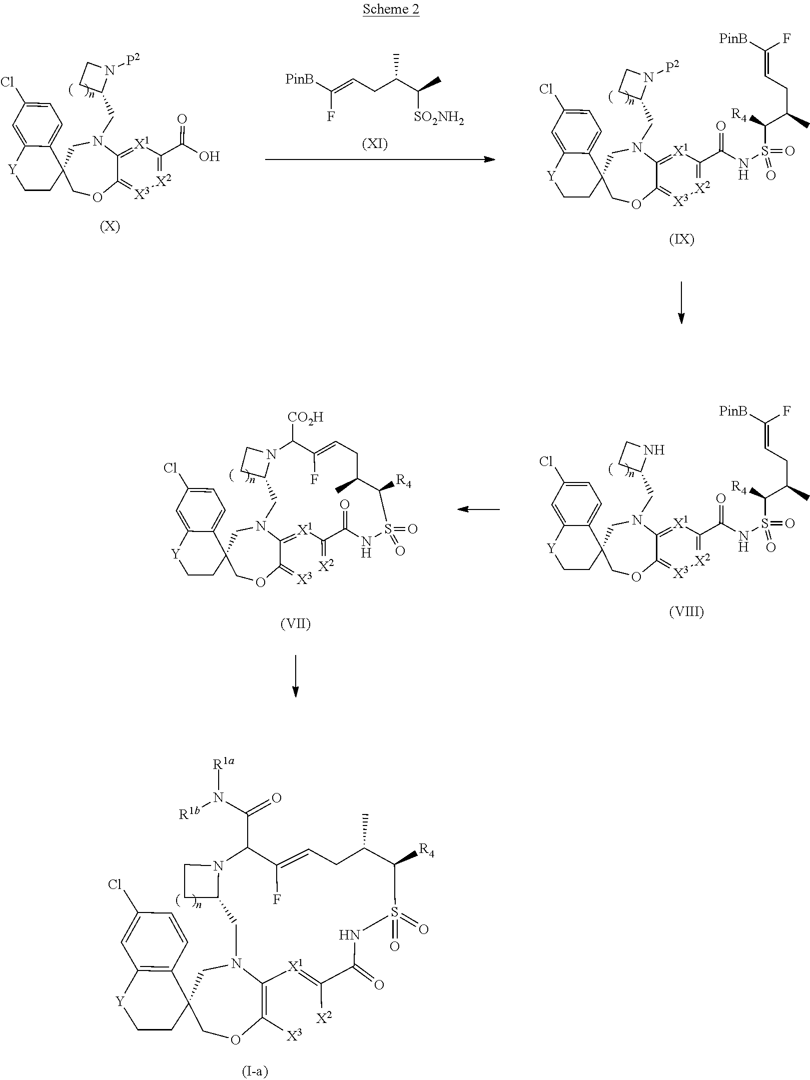

By reacting an intermediate of Formula (VII) with an amine in presence of a suitable coupling agent such as, for example, propanephosphonic acid anhydride, a suitable base such as, for example, triethylamine, in a suitable solvent such as, for example, dichloromethane or ethyl acetate, at a suitable temperature such as room temperature or 40° C.
Intermediates of Formula (VII) can be prepared by reacting an intermediate of Formula (VIII) with glyoxylic acid and a suitable base such as, for example, triethylamine, in a suitable solvent such as, for example, methanol or ethyl acetate, at a suitable temperature such as, for example, 40 or 60° C.
Intermediates of Formula (VIII) can be prepared by deprotecting an intermediate of Formula (IX), in which P² is a suitable protective group such as, for example, Boc, with a suitable deprotecting agent such as, for example, trifluoroacetic acid, in a suitable solvent such as, for example, dichloromethane, at a suitable temperature such as, for example, room temperature.
Intermediates of Formula (IX) can be prepared by coupling an intermediate of Formula (X) with an intermediate of Formula (XI) with a suitable coupling agent such as, for example, 1-(3-dimethylaminopropyl)-3-ethylcarbodiimide hydrochloride, in presence of an appropriate base such as, for example, triethylamine or 4-dimethylamino pyridine or a mixture thereof, in a suitable solvent such as, for example, dichloromethane, at a suitable temperature such as, for example, room temperature.

Alternatively and following Scheme 2, compounds of Formula (I-a) can be prepared directly from an intermediate of Formula (VIII) by reacting it with first with glyoxylic acid and a suitable base such as, for example, triethylamine, in a suitable solvent such as, for example, ethyl acetate, at a suitable temperature such as, for example, 40 or 60° C., and reacting the resulting intermediate of Formula (VII) in situ with a suitable amine in presence of a suitable coupling agent such as, for example, propanephosphonic acid anhydride, at a suitable temperature such as room temperature or 40° C.

Compounds of Formula (I-aa) wherein the variables are defined as in Formula (I)can be prepared according to Scheme 3,

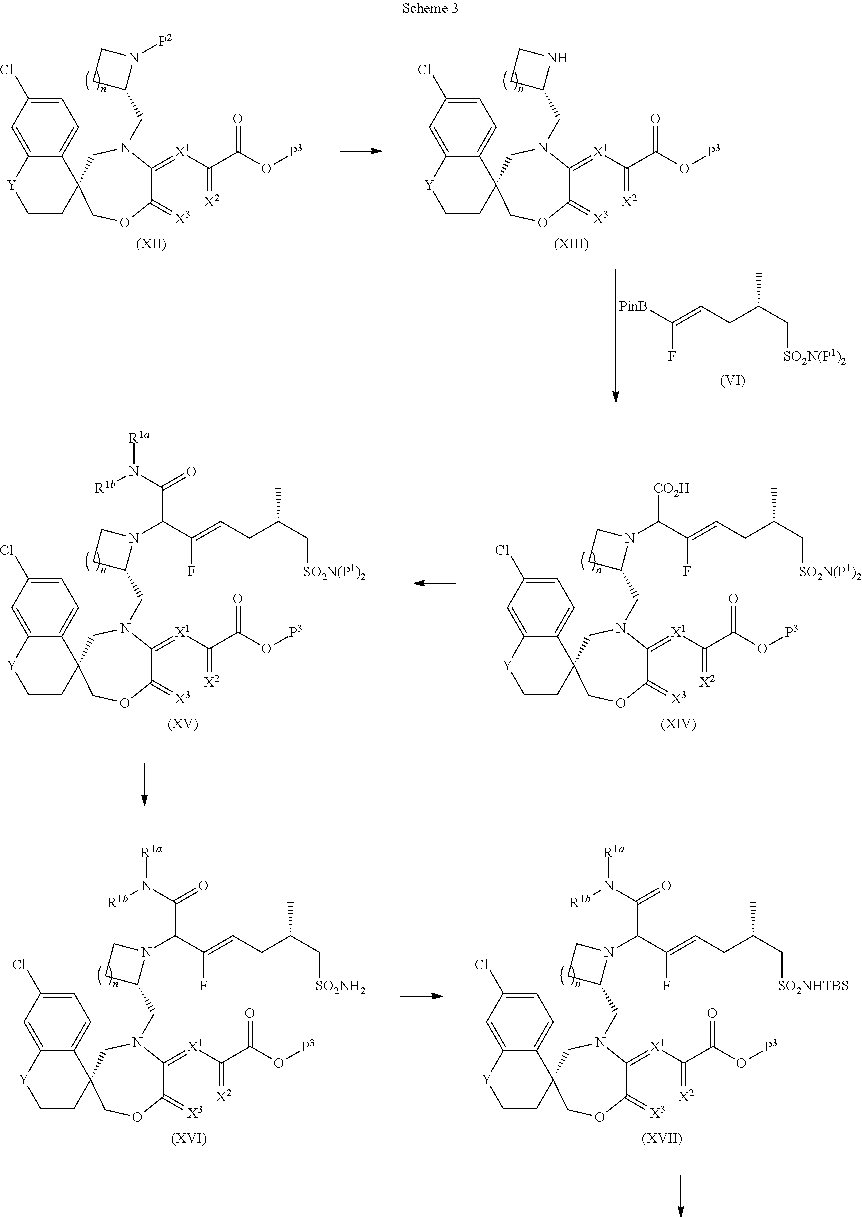

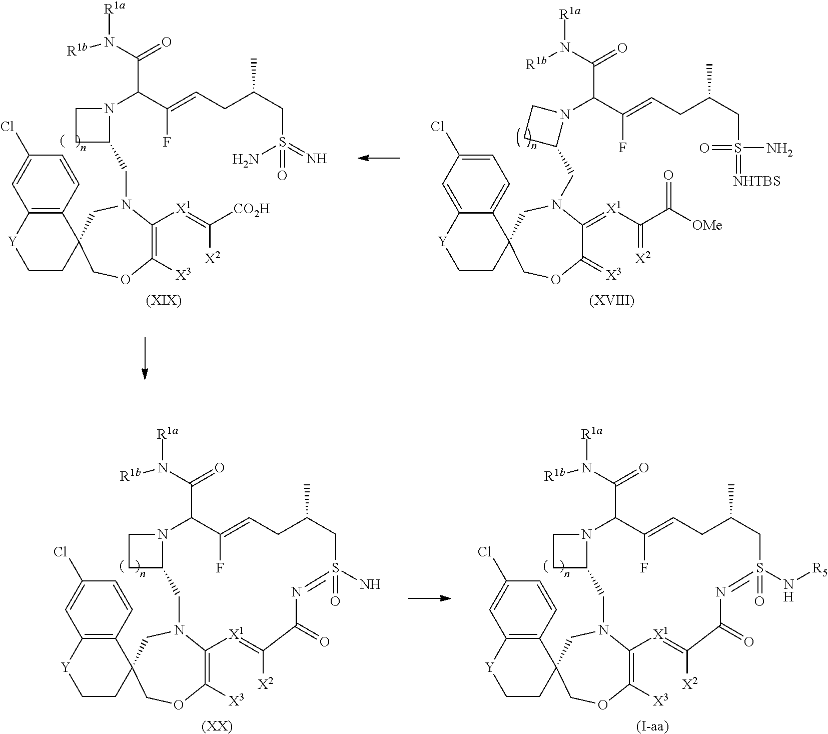

By reacting an intermediate of Formula (XX) with a carboxylic acid in a presence of a suitable amide formation reagent such as, for example, HATU, HBTU, DCC or T3P, in presence of a base such as, for example, triethylamine, in a suitable solvent such as, for example, DMF, at a suitable temperature such as room temperature.
Intermediates of Formula (XX) can be prepared by reacting an intermediate of Formula (XIX) with a suitable amide formation reagent such as, for example, diethyl cyanophosphonate, in a suitable solvent such as, for example, DMF, at a suitable temperature such as room temperature.
Intermediates of Formula (XIX) can be prepared by saponifying an intermediate of Formula (XVIII) in which P³ is a suitable protective group such as, for instance, methyl, in the presence of a suitable base such as, for example, lithium hydroxide, in a suitable solvent such as, for example, tetrahydrofuran or methanol or water or a mixture thereof, at a suitable temperature such as room temperature or 50° C.
Intermediates of Formula (XVIII) can be prepared by reacting an intermediate of Formula (XVII) with a suitable chlorinating agent such as, for example, chlorodiphenylphosphine, followed by treatment with a source of amine such as, for example, ammonia gas, in a suitable solvent such as, for example, THF, at a suitable temperature such as 0° C.
Intermediates of Formula (XVII) can be prepared by protection of an intermediate of Formula (XVI) with TBSCl in presence of a suitable base such as, for example, triethylamine, in a suitable solvent such as, for example, dichloromethane.
Intermediates of Formula (XVI) can be prepared by deprotection of an intermediate of Formula (XV) in which P¹ is a suitable protective group such as, for example, para-methoxybenzyl (PMB), with a suitable deprotecting agent such as, for example, trifluoroacetic acid, in a suitable solvent such as, for example, dichloromethane.
Intermediates of Formula (XV) can be prepared by reacting an intermediate of Formula (XIV) with an amine using a suitable coupling agent such as, for example, propanephosphonic acid anhydride, in presence of a suitable base such as, for example, triethylamine, in a suitable solvent such as, for example, dichloromethane or ethyl acetate, at a suitable temperature such as, for instance, room temperature or 40° C.
Intermediates of Formula (XIV) can be prepared by reacting an intermediate of Formula (XIII) with an intermediate of Formula (VI) (‘Pin’ means pinacol ester) and glyoxylic acid in a suitable solvent such as, for example, methanol, at a suitable temperature such as, for example, 40 or 60° C.
Intermediates of Formula (XIII) can be prepared by reacting an intermediate of Formula (XII) in which P² is a suitable protective group such as, for example, Boc, with a suitable deprotecting agent such as, for example, trifluoroacetic acid, as a suitable temperature such as, for example, room temperature.

Intermediates of Formula (V) and (X) wherein X¹, X², X³, Y and n are as defined in Formula (I), and wherein P² is a suitable protective group such as, for example, Boc, can be prepared according to Scheme 4,

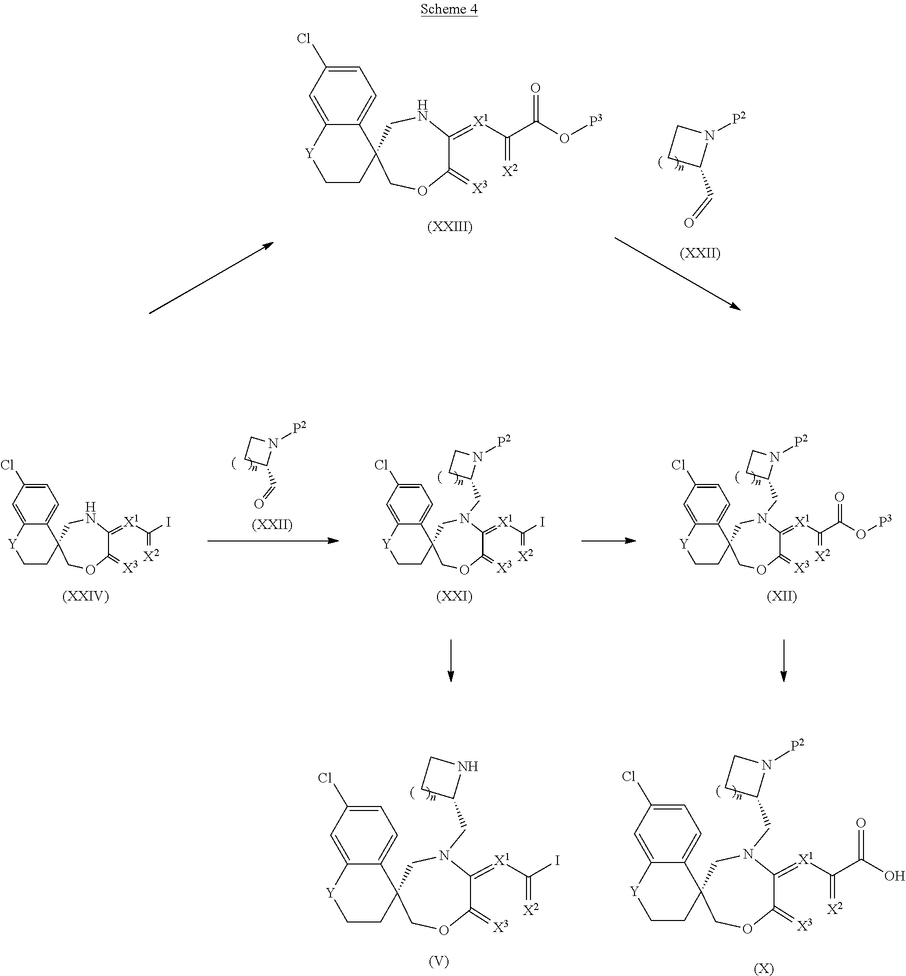

By deprotecting an intermediate of Formula (XII) in which P³ is a suitable protective group such as, for example methyl, with a suitable deprotection agent such as, for example, LiOH or NaOH, in a suitable solvent such as, for example, tetrahydrofuran or water or a mixture thereof, at a suitable temperature such as room temperature or 50° C.
Intermediates of Formula (XII) can be prepared by reacting an intermediate of Formula (XXI) in the presence of a suitable palladium catalyst such as, for example, [1,1′-bis(diphenylphosphino)ferrocene]palladium(II) dichloride, in a suitable solvent such as tetrahydrofuran, at a suitable temperature such as 80 or 100° C. and at a suitable CO pressure such as 30 bar.
Alternatively, intermediates of Formula (XII) can be prepared by reacting an intermediate of Formula (XXIII) with an intermediate of Formula (XXII) in presence of a suitable reducing agent such as, for example, sodium cyanoborohydride or sodium triacetoxy borohydride, in a suitable solvent such as, for example, dichloromethane or acetic acid or a mixture thereof, at a suitable temperature such as, for example, 0° C. or room temperature.
Intermediates of Formula (XXIII) can be prepared by reacting an intermediate of Formula (XXIV) in the presence of a suitable palladium catalyst such as, for example, [1,1′-bis(diphenylphosphino)ferrocene]palladium(II) dichloride, in a suitable solvent such as tetrahydrofuran, at a suitable temperature such as 80 or 100° C. and at a suitable CO pressure such as 30 bar.
Intermediates of Formula (V) can be prepared by reacting an intermediate of Formula (XXI) in which P² is a suitable protective group such as, for example, Boc, with a suitable deprotecting agent such as, for example, trifluoroacetic acid, at a suitable temperature such as, for example, room temperature.
Intermediates of Formula (XXI) can be prepared by reacting an intermediate of Formula (XXIV) with an intermediate of Formula (XXII) in presence of a suitable reducing agent such as, for example, sodium cyanoborohydride or sodium triacetoxy borohydride, in a suitable solvent such as, for example, dichloromethane or acetic acid or a mixture thereof, at a suitable temperature such as, for example, 0° C. or room temperature.

The synthesis of intermediate (XXII) in which P² is Boc corresponds with (CAS [200184-45-8] for n=1, CAS [69610-41-9] for n=2).

Intermediates of Formula (XXIV) wherein X¹, X², X³ are CH, and Y is as defined in Formula (I), can be prepared according to Scheme 5,

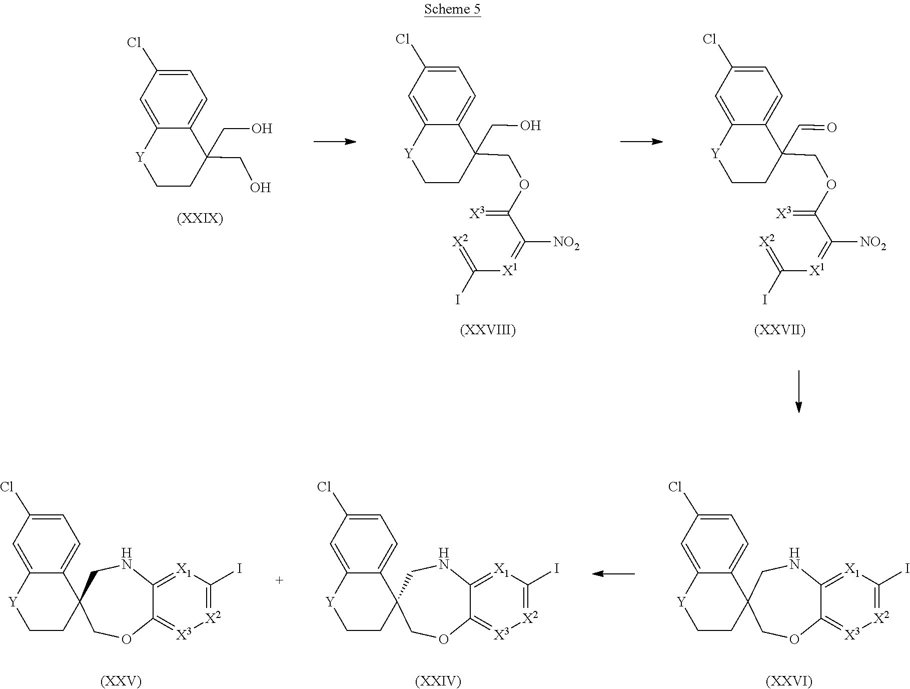

By separating the enantiomers of Formula (XXVI) using an appropriate separation technique such as, for example, chiral SFC.
Intermediate of Formula (XXVI) can be obtained by reacting an intermediate of Formula (XXVII) with a suitable reducing agent like, for instance, iron dust, in a suitable solvent such as, for instance, acetic acid, at a suitable temperature like, for instance, 70° C. It will be clear to a skilled person that the resulting aniline can condense intramolecularly with the aldehyde to an imine, which can be further reduced with a suitable reducing agent such as, for instance, sodium triacetoxyborohydride (NaBH(OAc)₃), in a suitable solvent such as, for instance, dichloromethane (DCM), at a suitable temperature such as, for instance, room temperature.
Intermediate of Formula (XXVII) can be prepared by reacting an intermediate of Formula (XXVIII) with a suitable oxidant such as, for instance, dimethylsulfoxide (DMSO) and oxalyl chloride, in the presence of a suitable base such as, for instance, triethylamine, in a suitable solvent such as, for instance, DCM, at a suitable temperature such as, for instance, −78° C. or room temperature (rt).
Intermediate of Formula (XXVIII) can be prepared by reacting an intermediate of Formula (XXIX) with 1-fluoro-4-iodo-2-nitrobenzene (CAS [364-75-0]) in presence of a suitable base such as, for instance, K₂CO₃, in a suitable solvent such as, for instance, acetonitrile, at a suitable temperature such as, for instance, 50° C.

Intermediate of Formula (XI) can be prepared according to Scheme 6,

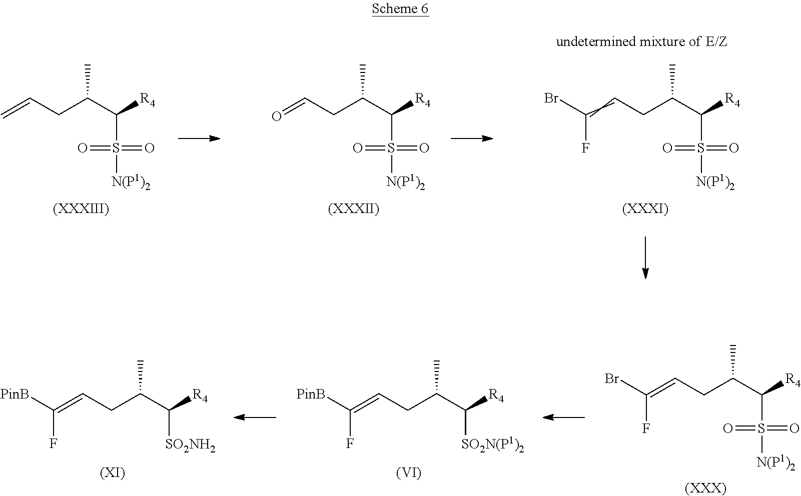

By deprotecting an intermediate of Formula (VI) wherein P¹ is a suitable protective group such as, for example, p-methoxybenzyl, with an appropriate deprotecting agent such as, for example, trifluoroacetic acid, in a suitable solvent such as, for example, dichloromethane, at a suitable temperature such as, for example, 0° C. or room temperature.
Intermediate of Formula (VI) can be prepared by reacting intermediate of Formula (XXX) with bis(pinacolato)diboron, in the presence of a suitable base such as, for example, potassium acetate, in the presence of a suitable catalyst such as, for example, [1,1′-bis(diphenylphosphino)ferrocene]palladium(II) dichloride dichloromethane complex (CAS [95464-05-4]) or [butylbis(tricyclo[3.3.1.1^{3,7}] dec-1-yl)phosphine](methanesulfonato-x0)[2′-(methylamino-κN)[1,1′-biphenyl]-2-yl-κC]-palladium (cataCXium Pd G4-CAS [2230788-67-5]), in a suitable solvent such as, for example, 1,4-dioxane, at a suitable temperature such as, for example, 60 or 95° C.
Intermediates of Formula (XXX) can be prepared by reacting an intermediate of Formula (XXXI) with a suitable base such as, for example, lithiumbis(trimethylsilyl)amide (LiH/IDS), in a suitable solvent such as, for example, tetrahydrofuran (THF), at a suitable temperature such as, for example 0° C.
Intermediates of Formula (XXXI) can be prepared by reacting an intermediate of Formula (XXXII) with fluorotribromomethane and triphenylphosphine, in the presence of a suitable dialkylzinc derivative such as, for example, diethylzinc, in a suitable solvent such as, for example, THF, at a suitable temperature such as, for example, room temperature.
Intermediates of Formula (XXXII) can be prepared either by ozonolysis of an intermediate of Formula (XXXIII) in a suitable solvent such as, for example, dichloromethane or methanol, at a suitable temperature such as, for example, −78° C., or by oxidation of an intermediate of Formula (XXXIII) with suitable reagents such as, for example, a catalytic amount of osmium tetroxide with sodium periodate, in a suitable solvent such as, for example, tetrahydrofuran and water mixture, at a suitable temperature such as, for example, room temperature.

Intermediate (XXXIII) in which P¹ is p-methoxybenzyl corresponds with (CAS [1883727-77-2]).

Alternatively Intermediates of Formula (XXXI) can be prepared according to Scheme 7,

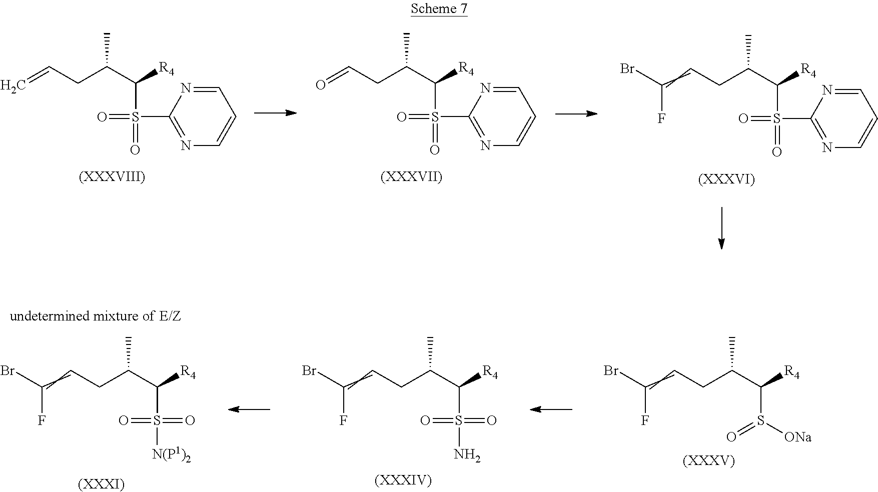

By protecting the intermediate of Formula (XXXIV) with a suitable protecting group such as, for example, p-methoxybenzyl chloride, in presence of a suitable base such as, for example, potassium carbonate, in a suitable solvent such as, for example, THF.
Intermediate of Formula (XXXIV) can be prepared by treatment of the intermediate of Formula (XXXV) with sodium acetate and hydroxylamine-O-sulfonic acid in a suitable solvent such as, for example, water, at a suitable temperature such as, for example, room temperature.
Intermediate of Formula (XXXV) can be prepared by treatment of the intermediate of Formula (XXXVI) with an appropriate base such as, for example, sodium methoxide, in a suitable solvent such as, for example, methanol, at a suitable temperature such as, for example, 0° C. or room temperature.
Intermediate of Formula (XXXVI) can be prepared by reacting the intermediate of Formula (XXXVII) with fluorotribromomethane and triphenylphosphine, in the presence of a suitable dialkylzinc derivative such as, for example, diethylzinc, in a suitable solvent such as, for example, THF, at a suitable temperature such as, for example, room temperature.
Intermediate of Formula (XXXVII) can be prepared either by ozonolysis of the intermediate of Formula (XXXVIII) in a suitable solvent such as, for example, dichloromethane or methanol, at a suitable temperature such as, for example, −78° C., or by oxidation of the intermediate of Formula (XXXVIII) with suitable reagents such as, for example, a catalytic amount of osmium tetroxide with sodium periodate, in a suitable solvent such as, for example, tetrahydrofuran and water mixture, at a suitable temperature such as, for example, room temperature.

Intermediate (XXXVIII) corresponds with (CAS [1638587-10-6]) Alternatively Intermediates of Formula (VI) can be prepared according to Scheme 8,

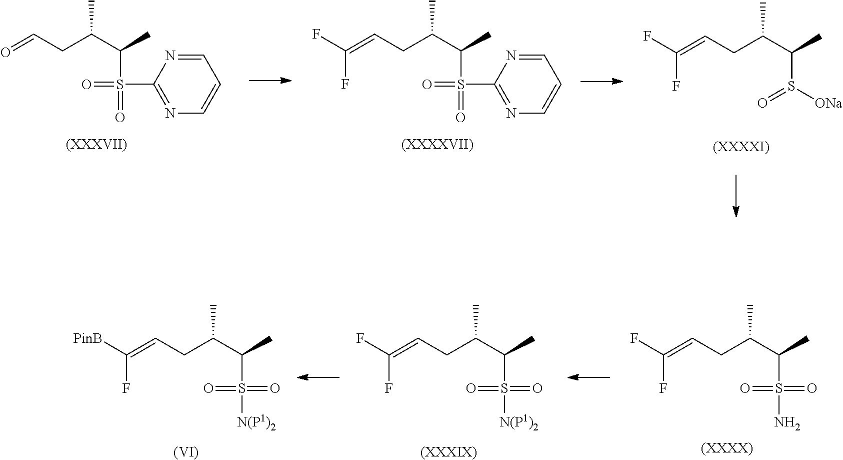

By reacting an intermediate of Formula (XXXIX) with bis(pinacolato)diboron and a copper catalyst such as, for example, copper chloride mixed with a phosphine ligand such as, for example, XantPhos, in presence of a base such as, for example, potassium tert-butoxide, and an alcohol such as, for example, methanol, in a suitable solvent such as, for example, 1,4-dioxane, at a suitable temperature such as, for example, 40° C.
Intermediates of Formula (XXXIX) can be prepared by protecting the intermediate of Formula (XXXX) with a suitable protecting group such as, for example, p-methoxybenzyl chloride, in presence of a suitable base such as, for example, potassium carbonate, in a suitable solvent such as, for example, THF.
Intermediate of Formula (XXXX) can be prepared by treatment of the intermediate of Formula (XXXXI) with sodium acetate and hydroxylamine-O-sulfonic acid in a suitable solvent such as, for example, water, at a suitable temperature such as, for example, room temperature.
Intermediate of Formula (XXXXI) can be prepared by treatment of the intermediate of Formula (XXXXII) with an appropriate base such as, for example, sodium methoxide, in a suitable solvent such as, for example, methanol, at a suitable temperature such as, for example, 0° C. or room temperature.
Intermediate of Formula (XXXXII) can be prepared by reacting the intermediate of Formula (XXXVII) with sodium chlorodifluoroacetate and triphenylphosphine, in a suitable solvent such as, for example, DMF, at a suitable temperature such as, for example, room temperature.

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of Formula (I) may be synthesized in the form of racemic mixtures of enantiomers which can be separated from one another following art-known resolution procedures. The racemic compounds of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (Boc), benzyloxycarbonyl (CBz) and 9-fluorenylmethyleneoxycarbonyl (Fmoc). The need for such protection is readily determined by one skilled in the art. For a general description of protecting groups and their use, see T. W. Greene and P. G. M. Wuts, Protective Groups in Organic Synthesis, 4th ed., Wiley, Hoboken, N.J., 2007.

Pharmacology of Compounds

It has been found that the compounds of the present invention inhibit one of more MCL-1 activities, such as MCL-1 antiapoptotic activity.

An MCL-1 inhibitor is a compound that blocks one or more MCL-1 functions, such as the ability to bind and repress proapoptotic effectors Bak and Bax or BH3 only sensitizers such as Bim, Noxa or Puma.

The compounds of the present invention can inhibit the MCL-1 pro-survival functions. Therefore, the compounds of the present invention may be useful in treating and/or preventing, in particular treating, diseases that are susceptible to the effects of the immune system such as cancer.

In another embodiment of the present invention, the compounds of the present invention exhibit anti-tumoral properties, for example, through immune modulation.

In an embodiment, the present invention is directed to methods for treating and/or preventing a cancer, wherein the cancer is selected from those described herein, comprising administering to a subject in need thereof (preferably a human), a therapeutically effective amount of a compound of Formula (I), or pharmaceutically acceptable salt, or a solvate thereof.

In an embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of acute lymphoblastic leukemia (ALL), acute myeloid leukemia (AML), B cells acute lymphoblastic leukemia, B-cell chronic lymphocytic leukemia (CLL), bladder cancer, breast cancer, chronic lymphocytic leukemia, chronic myeloid leukemia, colon adenocarcinoma, diffuse large B cell lymphoma, esophageal cancer, follicular lymphoma, gastric cancer, head and neck cancer (including, but not limited to head and neck squamous cell carcinoma), hematopoietic cancer, hepatocellular carcinoma, Hodgkin lymphoma, liver cancer, lung cancer (including but not limited to lung adenocarcinoma), lymphoma, medulloblastoma, melanoma, monoclonal gammopathy of undetermined significance, multiple myeloma, myelodysplastic syndromes, myelofibrosis, myeloproliferative neoplasms, ovarian cancer, ovarian clear cell carcinoma, ovarian serous cystadenoma, pancreatic cancer, polycythemia vera, prostate cancer, rectum adenocarcinoma, renal cell carcinoma, smoldering multiple myeloma, T cell acute lymphoblastic leukemia, T cell lymphoma, and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is preferably selected from the group consisting of acute lymphoblastic leukemia (ALL), acute myeloid leukemia (AML), B cells acute lymphoblastic leukemia, B-cell chronic lymphocytic leukemia (CLL), breast cancer, chronic lymphocytic leukemia, chronic myeloid leukemia, diffuse large B cell lymphoma, follicular lymphoma, hematopoietic cancer, Hodgkin lymphoma, lung cancer (including, but not limited to lung adenocarcinoma) lymphoma, monoclonal gammopathy of undetermined significance, multiple myeloma, myelodysplastic syndromes, myelofibrosis, myeloproliferative neoplasms, smoldering multiple myeloma, T cell acute lymphoblastic leukemia, T cell lymphoma and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of adenocarcinoma, benign monoclonal gammopathy, biliary cancer (including, but not limited to, cholangiocarcinoma), bladder cancer, breast cancer (including, but not limited to, adenocarcinoma of the breast, papillary carcinoma of the breast, mammary cancer, medullary carcinoma of the breast), brain cancer (including, but not limited to, meningioma), glioma (including, but not limited to, astrocytoma, oligodendroglioma; medulloblastoma), bronchus cancer, cervical cancer (including, but not limited to, cervical adenocarcinoma), chordoma, choriocarcinoma, colorectal cancer (including, but not limited to, colon cancer, rectal cancer, colorectal adenocarcinoma), epithelial carcinoma, endothelial sarcoma (including, but not limited to, Kaposi's sarcoma, multiple idiopathic hemorrhagic sarcoma), endometrial cancer (including, but not limited to, uterine cancer, uterine sarcoma), esophageal cancer (including, but not limited to, adenocarcinoma of the esophagus, Barrett's adenocarinoma), Ewing sarcoma, gastric cancer (including, but not limited to, stomach adenocarcinoma), gastrointestinal stromal tumor (GIST), head and neck cancer (including, but not limited to, head and neck squamous cell carcinoma), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, kidney cancer (including, but not limited to, nephroblastoma a.k.a. Wilms' tumor, renal cell carcinoma), liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES), ovarian cancer (including, but not limited to, cystadenocarcinoma, ovarian embryonal carcinoma, ovarian adenocarcinoma), pancreatic cancer (including, but not limited to, pancreatic andenocarcinoma, intraductal papillary mucinous neoplasm (IPMN), Islet cell tumors), prostate cancer (including, but not limited to, prostate adenocarcinoma), skin cancer (including, but not limited to, squamous cell carcinoma (SCC), keratoacanthoma (KA), melanoma, basal cell carcinoma (BCC)) and soft tissue sarcoma (e.g. malignant fibrous histiocytoma (MFH), liposarcoma, malignant peripheral nerve sheath tumor (MPNST), chondrosarcoma, fibrosarcoma, myxosarcoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of benign monoclonal gammopathy, breast cancer (including, but not limited to, adenocarcinoma of the breast, papillary carcinoma of the breast, mammary cancer, medullary carcinoma of the breast), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), and prostate cancer (including, but not limited to, prostate adenocarcinoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of prostate, lung, pancreatic, breast, ovarian, cervical, melanoma, B-cell chronic lymphocytic leukemia (CLL), acute myeloid leukemia (AML), and acute lymphoblastic leukemia (ALL).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is multiple myeloma.

The compounds according to the present invention or pharmaceutical compositions comprising said compounds, may also have therapeutic applications in combination with immune modulatory agents, such as inhibitors of the PD1/PDL1 immune checkpoint axis, for example antibodies (or peptides) that bind to and/or inhibit the activity of PD-1 or the activity of PD-L1 and or CTLA-4 or engineered chimeric antigen receptor T cells (CART) targeting tumor associated antigens.

The compounds according to the present invention or pharmaceutical compositions comprising said compounds, may also be combined with radiotherapy or chemotherapeutic agents (including, but not limited to, anti-cancer agents) or any other pharmaceutical agent which is administered to a subject having cancer for the treatment of said subject's cancer or for the treatment or prevention of side effects associated with the treatment of said subject's cancer.

The compounds according to the present invention or pharmaceutical compositions comprising said compounds, may also be combined with other agents that stimulate or enhance the immune response, such as vaccines.

In an embodiment, the present invention is directed to methods for treating and/or preventing a cancer (wherein the cancer is selected from those described herein) comprising administering to a subject in need thereof (preferably a human), a therapeutically effective amount of co-therapy or combination therapy; wherein the co-therapy or combination therapy comprises a compound of Formula (I) of the present invention and one or more anti-cancer agent(s) selected from the group consisting of (a) immune modulatory agent (such as inhibitors of the PD1/PDL1 immune checkpoint axis, for example antibodies (or peptides) that bind to and/or inhibit the activity of PD-1 or the activity of PD-L1 and or CTLA-4); (b) engineered chimeric antigen receptor T cells (CART) targeting tumor associated antigens; (c) radiotherapy; (d) chemotherapy; and (e) agents that stimulate or enhance the immune response, such as vaccines.

The present invention is directed to compounds of Formula (I) and pharmaceutically acceptable salts, and solvates thereof, for use as a medicament.

The present invention is directed to compounds of Formula (I) and pharmaceutically acceptable salts, and solvates thereof, for use in the inhibition of MCL-1 activity.

As used herein, unless otherwise noted, the term “anti-cancer agents” shall encompass “anti-tumor cell growth agents” and “anti-neoplastic agents”.

The present invention is directed to compounds of Formula (I) and pharmaceutically acceptable salts, and solvates thereof, for use in treating and/or preventing diseases (preferably cancers) mentioned above.

The present invention is directed to compounds of Formula (I) and pharmaceutically acceptable salts, and solvates thereof, for treating and/or preventing diseases (preferably cancers) mentioned above.

The present invention is directed to compounds of Formula (I) and pharmaceutically acceptable salts, and solvates thereof, for treating and/or preventing, in particular for treating, a disease, preferably a cancer, as described herein (for example, multiple myeloma).

The present invention is directed to compounds of Formula (I) and pharmaceutically acceptable salts, and solvates thereof, for use in treating and/or preventing, in particular for treating, a disease, preferably a cancer, as described herein (for example, multiple myeloma).

The present invention is directed to compounds of Formula (I) and pharmaceutically acceptable salts, and solvates thereof, for treating and/or preventing, in particular for treating, MCL-1 mediated diseases or conditions, preferably cancer, more preferably a cancer as herein described (for example, multiple myeloma).

The present invention is directed to compounds of Formula (I) and pharmaceutically acceptable salts, and solvates thereof, for use in treating and/or preventing, in particular for use in treating, MCL-1 mediated diseases or conditions, preferably cancer, more preferably a cancer as herein described (for example, multiple myeloma).

The present invention relates to compounds of Formula (I) and pharmaceutically acceptable salts, and solvates thereof, for the manufacture of a medicament.

The present invention relates to compounds of Formula (I) and pharmaceutically acceptable salts, and solvates thereof, for the manufacture of a medicament for the inhibition of MCL-1.

The present invention relates to compounds of Formula (I) and pharmaceutically acceptable salts, and solvates thereof, for the manufacture of a medicament for treating and/or preventing, in particular for treating, a cancer, preferably a cancer as herein described. More particularly, the cancer is a cancer which responds to inhibition of MCL-1 (for example, multiple myeloma).

The present invention is directed to compounds of Formula (I) and pharmaceutically acceptable salts, and solvates thereof, for the manufacture of a medicament for treating and/or preventing, in particular for treating, any one of the disease conditions mentioned hereinbefore.

The present invention is directed to compounds of Formula (I) and pharmaceutically acceptable salts, and solvates thereof, for the manufacture of a medicament for treating and/or preventing any one of the disease conditions mentioned hereinbefore.

The compounds of Formula (I) and pharmaceutically acceptable salts, and solvates thereof, can be administered to subjects, preferably humans, for treating and/or preventing of any one of the diseases mentioned hereinbefore.

In view of the utility of the compounds of Formula (I) and pharmaceutically acceptable salts, and solvates thereof, there is provided a method of treating subjects, preferably mammals such as humans, suffering from any of the diseases mentioned hereinbefore; or a method of slowing the progression of any of the diseases mentioned hereinbefore in subject, humans; or a method of preventing subjects, preferably mammals such as humans, from suffering from any one of the diseases mentioned hereinbefore.

Said methods comprise the administration, i.e. the systemic or topical administration, preferably oral or intravenous administration, more preferably oral administration, of an effective amount of a compound of Formula (I) or a pharmaceutically acceptable salt, or a solvate thereof, to subjects such as humans.

One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. In an embodiment, a therapeutically effective daily amount may be from about 0.005 mg/kg to 100 mg/kg.

The amount of a compound according to the present invention, also referred to herein as the active ingredient, which is required to achieve a therapeutic effect may vary on case-by-case basis, for example with the specific compound, the route of administration, the age and condition of the recipient, and the particular disorder or disease being treated. The methods of the present invention may also include administering the active ingredient on a regimen of between one and four intakes per day. In these methods of the present invention, the compounds according to the invention are preferably formulated prior to administration.

The present invention also provides compositions for treating and/or preventing the disorders (preferably a cancer as described herein) referred to herein. Said compositions comprise a therapeutically effective amount of a compound of Formula (I), or a pharmaceutically acceptable salt, or a solvate thereof, and a pharmaceutically acceptable carrier or diluent.

While it is possible for the active ingredient (e.g. a compound of the present invention) to be administered alone, it is preferable to administer it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of the present invention may be prepared by any methods well known in the art of pharmacy, for example, using methods such as those described in, for example, Gennaro et al. Remington's Pharmaceutical Sciences (18^{th} ed., Mack Publishing Company, 1990, see especially Part 8. Pharmaceutical preparations and their Manufacture).

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, in an embodiment, the present invention is directed to a product comprising, as a first active ingredient a compound according to the invention and as further, as an additional active ingredient one or more anti-cancer agent(s), as a combined preparation for simultaneous, separate or sequential use in the treatment of patients suffering from cancer.

The one or more other anti-cancer agents and the compound according to the present invention may be administered simultaneously (e.g. in separate or unitary compositions) or sequentially, in either order. In an embodiment, the two or more compounds are administered within a period and/or in an amount and/or a manner that is sufficient to ensure that an advantageous or synergistic effect is achieved. It will be appreciated that the preferred method and order of administration and the respective dosage amounts and regimes for each component of the combination will depend on the particular other anti-cancer agent and the compound of the present invention being administered, their route of administration, the particular condition, in particular tumor, being treated and the particular host being treated.

The following examples further illustrate the present invention.

### Examples

Several methods for preparing the Compounds of this invention are illustrated in the following examples. Unless otherwise noted, all starting materials were obtained from commercial suppliers and used without further purification, or alternatively can be synthesized by a skilled person by using published methods.

TABLE 1
Abbreviations

| Abbreviation | Meaning |
|---|---|
| AcOH | acetic acid |
| Boc | tert-butyloxycarbonyl |
| Celite ® | diatomaceous earth |
| DAST | (diethylamino)sulfur trifluoride |
| DBU | 1,8-diazabicyclo[5.4.0]undec-7-ene |
| DCE | 1,2-dichloroethane |
| DCM | dichloromethane |
| DEAD | diethyl azodicarboxylate |
| DECP | diethyl cyanophosphonate |
| DIAD | diisopropyl azodicarboxylate |
| DIBALH or DIBAH | di-isobutylaluminiumhydride |
| Dicalite ® | diatomaceous earth |
| DIPE | diisopropyl ether |
| DIPEA | N,N-diisopropylethylamine |
| DMAP | 4-dimethylaminopyridine |
| DME | 1,2-Dimethoxyethane |
| DMF | N,N-dimethylformamide |
| DMSO | dimethyl sulfoxide |
| DPPA | diphenylphosphoryl azide |
| EDC•HCl or EDCI | N-(3-dimethylaminopropyl)-N′-ethylcarbodiimide |
| | hydrochloride |
| eq. | equivalent(s) |
| Et | ethyl |
| EtOAc or AcOEt | ethyl acetate |
| EtOH | ethanol |
| g | gram(s) |
| h | hour(s) |
| HBTU | 2-(1H-Benzotriazole-1-yl)-1,1,3,3- |
| | tetramethyluronium hexafluorophosphate |
| HPLC | high performance liquid chromatography |
| iPrNH₂ | isopropylamine |
| L | liter(s) |
| LiHMDS | lithium bis(trimethylsilyl)amide |
| M | molar |
| Me | methyl |
| MeOH | methanol |
| MeOH | methanol |
| mg | milligram(s) |
| min | minute(s) |
| uL or μL | microliter(s) |
| mL | millilitre(s) |
| mmol | millimolar |
| MTBE | Methyl tert-butyl ether |
| N | normal |
| NaBH(OAc)₃ | sodium triacetoxyborohydride |
| NaOAc | sodium acetate |
| PBu₃ | tributylphosphine |
| PdCl₂(dppf) | [1,1′- |
| | bis(diphenylphosphino)ferrocene]dichloro- |
| | palladium(II) |
| pin | pinacol ester |
| PMB | p-methoxybenzyl |
| PPh₃ or Ph₃P | triphenylphosphine |
| Prep | preparative |
| PTSA | p-toluenesulfonic acid |
| quant. | quantitative yield |
| RM | reaction mixture |
| RP | reversed phase |
| RP | reversed phase |
| Rₜ or Rt | retention time (in minutes) |
| SFC | supercritical fluid chromatography |
| TBDMS | tert-butyldimethylsilyl |
| tBuOK or KOtBu | potassium tert-butoxide |
| TFA | trifluoroacetic acid |
| THF | tetrahydrofuran |
| TEMPO | (2,2,6,6-Tetramethylpiperidin-1-yl)oxyl |
| Xantphos | 4,5-Bis(diphenylphosphino)-9,9-dimethylxanthene |
| Tⱼ | Temperature of the jacket |

As understood by a person skilled in the art, Compounds synthesized using the protocols as indicated may contain residual solvent or minor impurities.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

In case stereobonds are shown in the structures of the intermediates and compounds of this invention, this means that the stereochemistry is absolute and determined, irrespective of the fact if stereodescriptors were also added or not.

Preparation of Intermediates

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

Intermediate 1

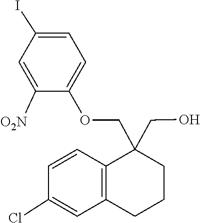 [SMILES: O=[N+]([O-])c1cc(I)ccc1OCC1(CO)CCCc2cc(Cl)ccc21]

This reaction was performed in two batches.

For each batch, (6-chloro-1,2,3,4-tetrahydronaphthalene-1,1-diyl)dimethanol (CAS [1883726-74-6]) (300 g, 1.32 mol) and 1-fluoro-4-iodo-2-nitrobenzene (353 g, 1.32 mol) were dissolved in acetonitrile (1.4 L). K₂CO₃ (549 g, 3.97 mol) was added to the reaction mixture and it was stirred at 50° C. for 16 h. The reaction mixture was filtered and the filtrate was evaporated. The two batches were then combined, and the residue was purified by column chromatography (SiO₂, Petroleum ether: dichloromethane=3/1 to petroleum ether/EtOAc=1:1). Intermediate 1 was obtained as a yellow oil (600 g, 48% yield).

Intermediate 2

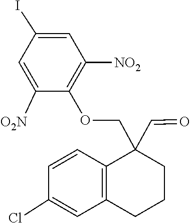 [SMILES: O=CC1(COc2c([N+](=O)[O-])cc(I)cc2[N+](=O)[O-])CCCc2cc(Cl)ccc21]

This reaction was performed in three batches.

For each batch, DMSO (99.0 g, 1.27 mol) was added to a solution of (COCl)₂ (161 g, 1.27 mol) in DCM (2.4 L) at −78° C. The reaction mixture was stirred at −78° C. for 15 min. Intermediate 1 (200 g, 422 mmol) in DCM (0.90 L) was then added at −78° C. and stirring was continued for 30 min. at −78° C. Et₃N (214 g, 2.11 mol) was added at −78° C. and the reaction mixture was allowed to warm to room temperature. Stirring was continued at room temperature for 1.5 h. Aqueous NaHCO₃ (1 L) was added and the mixture was extracted with DCM (0.5 L×2). The three batches were then combined and evaporated to yield Intermediate 2 (560 g) as a yellow solid, used without further purification.

Intermediate 3 and Intermediate 3b

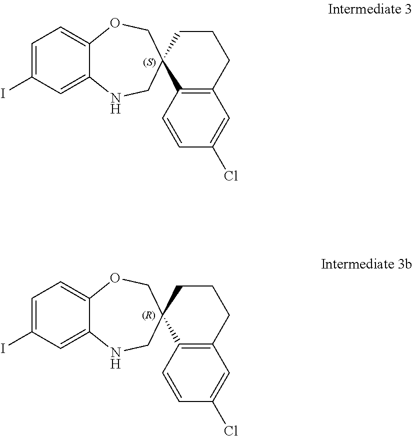

This reaction was performed in three batches.

Iron (153 g, 2.75 mol) was added to a solution of Intermediate 2 (185 g, 392 mmol) in AcOH (2.5 L) at 70° C. and the reaction mixture was stirred at 70° C. for 3 h. The solvent was evaporated and DCE (1.9 L) was added to the residue. NaBH(OAc)₃ (333 g, 1.57 mol) was then added portionwise at 0° C. Stirring was continued at room temperature for 1 h. The three batches were combined. Citric acid (10% solution in water, 5 L) was added and the mixture was extracted with DCM (2 L×2). The combined organic layer was evaporated. The residue was purified by SFC (column: DAICEL CHIRALPAK AD (250 mm*50 mm, 10 μm); mobile phase A: CO₂; mobile phase B: [0.1% NH₃H₂O in EtOH]) to afford Intermediate 3 (95.2 g, 40%) and its enantiomer intermediate 3b (105.1 g, 44%), both as yellow solids.

Intermediate 4

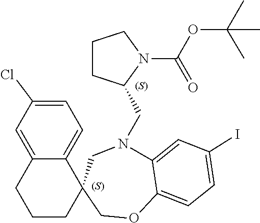

Intermediate 3 (13.197 g, 31 mmol) and N-Boc-L-prolinal (18.53 g, 3 eq.) were dissolved in CH₂Cl₂ (150 mL) and then AcOH (35.5 mL, 20 eq.) was added. The mixture was stirred for 30 min at room temperature and then cooled down to 0° C. Then NaBH(OAc)₃ (19.71 g, 3 eq.) was added portionwise. After addition the reaction mixture was stirred at room temperature for 18 hours. The reaction mixture was poured out portionwise into a cooled (0° C.) solution of NaOH (31 g) in 620 mL water. After addition the mixture was diluted with CH₂Cl₂ and water. The organic layer was separated, washed with water, dried with MgSO₄, filtered and the solvents of the filtrate were evaporated. The residue was purified by flash chromatography on silica gel (eluent: CH₂Cl₂) The fractions containing product were combined and the solvents were evaporated. This residue was purified again by column chromatography (gradient ethylacetate/hexane) to give Intermediate 4 (12.2 g, 64%).

Intermediate 5

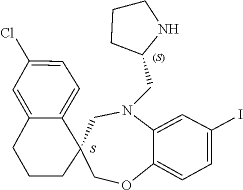

To a stirred solution of Intermediate 4 (12.2 g, 20.0 mmol) anhydrous DCM (200 mL) was added TFA (20 mL, 1.49 g/mL, 261 mmol). The resulting mixture was stirred at room temperature during 20 h. Most of the solvents were evaporated under reduced pressure at low temperature (<35° C.). DCM was added. Then, the mixture was carefully quenched with sat. NaHCO₃ sol. until pH 8-9. The organic layer was extracted, washed with brine (100 mL), dried (Na₂SO₄), filtered and evaporated under reduced pressure.

The crude product was purified by column chromatography with DCM/MeOH (96:4) to afford Intermediate 5 (8.14 g, 80%) as a pale yellow foam.

Intermediate 6

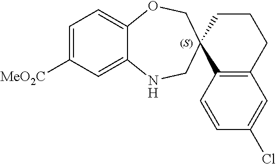

An autoclave was charged with Intermediate 3 (1 g, 0.0023 mol) and 1 mL of triethylamine in 50 mL of MeOH. [1,1′-bis(diphenylphosphino)ferrocene]dichloropalladium(II) (17.2 mg, 0.000023 mol) was added and the setup was closed. Then this was flushed one time with carbon monoxide and then pressurised with ca 30 bar CO. The ensuing reaction mixture was heated 20 h at 100° C. The mixture was evaporated and purified on silica gel, eluent: 0-2% MeOH in DCM to give Intermediate 6 (720 mg, yield 86%) as a white solid.

Alternatively, Intermediate 6 was prepared by using an analogous procedure as Intermediate 3 using methyl 4-fluoro-3-nitrobenzoate as precursor and was isolated as a clear oil.

Intermediate 7

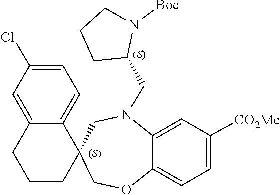

Intermediate 6 (50 g, 140 mmol) and N-Boc-L-prolinal (60 g, 301 mmol) were dissolved in DCM (600 mL) and then stirred at 1° C. AcOH (152 mL) was added at 1° C. The mixture was stirred for 10 minutes at 1° C. Then NaBH(OAc)₃ (63 g, 297 mmol)) was added portion wise over a period of 6 hours. After addition the reaction mixture was stirred at 1° C. for 12 hours, then at 15° C. for 40 hours. The reaction mixture was poured out portion wise into a cooled (0° C.), stirred solution of NaOH (50% in H₂O) (280 g, 3500 mmol) in 2 L water and 1 L DCM. Then the mixture was separated, the aqueous layer was extracted with DCM (2x). The combined organic layers were separated, washed with water, dried with MgSO₄, filtered and concentrated in vacuo. This was purified by flash chromatography (3 times a 300 g column) using 0-30% EtOAc in heptane to yield Intermediate 7 (76 g, quant.) as a white foam (76 g, 100%).

Alternatively, an autoclave was charged with Intermediate 4 (6.7 g, 0.011 mol) and triethylamine (4.872 mL, 0.73 g/mL, 0.0351 mol) in 174 mL of THE and 940 mL of MeOH, then [1,1′-bis(diphenylphosphino)ferrocene]dichloropalladium(II) (1.183 g, 0.00162 mol) was added and the setup was closed. Then this was flushed one time with carbon monoxide and then pressurised with 30 bar of CO. The ensuing reaction mixture was heated 20 h at 100° C. The mixture was evaporated under reduced pressure and directly purified on silica gel, eluent: 0-2% MeOH in DCM to yield Intermediate 7 (5.25 g, 88%) as a white solid.

Intermediate 8

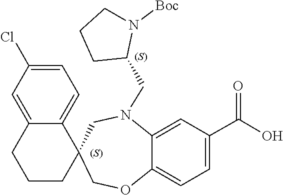

To the solution of Intermediate 7 (5.25 g, 9.70 mmol) in THE (158 mL), water (158 mL) and MeOH (30 mL) was added LiOH (1.13 g, 46.98 mmol), this was stirred at 50° C. for 16 h. The reaction mixture was cooled (10° C.) and acidified (pH=3-4) with 1 N HCl aqueous solution, then extracted with DCM. The organic layer was dried over MgSO₄, concentrated under reduced pressure to give Intermediate 8 (5.34 g, quant.) as an off-white foam.

Intermediate 9

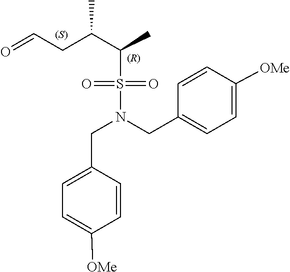

(2S,3R)-N,N-bis[(4-methoxyphenyl)methyl]-3-methyl-5-hexene-2-sulfonamide (CAS [1883727-77]) (1 g, 2.275 mmol) was dissolved in a mixture of DCM (12.5 mL) and MeOH (12.5 mL) and the resulting mixture was cooled to −78° C. Ozone (109 mg, 1 eq.) was subsequently bubbled through the reaction mixture until a blue persistent color was observed (5 min). Nitrogen was then bubbled through the solution (still at −78° C.) to remove the blue color and this was followed by addition of PPh₃ (2.98 g, 5 eq). Once the addition was complete, the reaction was left stirring at −78° C. for 1 h. The reaction mixture was then slowly allowed to warm to room temperature and was stirred for 1 h. The heterogenous mixture was filtered through a pad of Dicalite®®. The pad was thoroughly washed with DCM. The filtrate was concentrated under reduced pressure to give a green oil that was purified by flash column chromatography on silica gel (heptane:EtOAc-1:0 to 3:1) to give Intermediate 9 (920 mg, yield 87%) as a colorless oil.

Intermediate 10

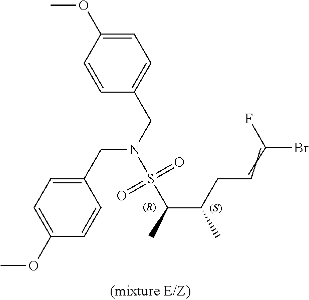

Diethyl zinc (27 mL, 1 M in heptane, 1.4 eq.) was added over 4 h via a syringe pump at room temperature to a stirred solution of Intermediate 9 (8 g, 19.07 mmol), fluorotribromomethane (2.8 mL, 1.5 eq.) and triphenylphosphine (7.5 g, 1.5 eq.) in dry THE (150 mL). Immediately after addition, the yellow solution was quenched with MeOH (20 mL) and evaporated under reduced pressure. The residue was purified by column chromatography on silica gel with heptane/EtOAc (1:0 to 4:1) to afford Intermediate 10 (8.4 g, 86%) as a clear oil (48/52 mixture of Z/E isomers).

Intermediate 11

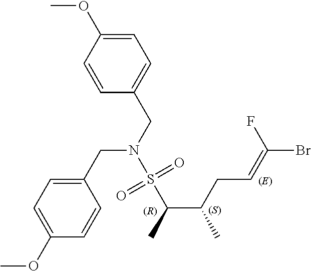

LiHMDS (60 mL, 1 M in THF, 60 mmol) was added over 14 h via a syringe pump to a cold (0° C.) stirred solution of Intermediate 10 (28.0 g, 54.4 mmol) in dry THE (350 mL). Following the addition, the mixture was further stirred for 30 min at 0° C. before quenching with water (100 mL), then EtOAc (150 mL) was added. The layers were separated and the organic layer was washed with brine (100 mL), dried (MgSO₄), filtered, and evaporated under reduced pressure. The residue was purified by column chromatography on silica gel using heptane/EtOAc (1:0 to 4:1) to afford Intermediate 11 (13.72 g, yield 49%) as a clear oil with >99:1 E/Z ratio.

Intermediate 12

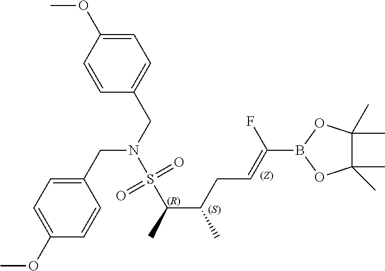

Intermediate 11 (15 g, 29.157 mmol), bis(pinacolato)diboron (15 g, 59.1 mmol) and KOAc (6 g, 61 mmol) were charged in an EasyMax reactor which was purged with nitrogen. Then, 290 mL of anhydrous 1,4-dioxane was added and nitrogen was bubbled through the mixture for few minutes. CataCXium Pd G4 (CAS [2230788-67-5]) (217 mg, 0.292 mmol) was added and the mixture was stirred at 60° C. under nitrogen flow for 24 h. After cooling to room temperature, the mixture was filtered through Celite® and washed with EtOAc. The filtrate was evaporated under reduced pressure. The crude was subjected to column chromatography with Heptane/EtOAc (1:0 to 85:15). The fractions corresponding to the product were collected and evaporated under reduced pressure to get the desired compound as a pale brownish oil, which was directly dissolved in EtOAc. Circa 2 g of SiliaMetS® metal scavenger (Silicycle, 0.61 mmol/g regarding catalyst loading) was added and the mixture was stirred 2 h at room temperature. The mixture was filtered over Celite® and washed with EtOAc. The filtrate was evaporated under reduced pressure to get Intermediate 12 (15.9 g, yield 87%) as a yellow oil.

Intermediate 13

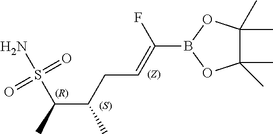

TFA (60 mL, 1.49 g/mL, 784 mmol) was added dropwise to a stirred solution of Intermediate 12 (15.5 g, 28 mmol) in 140 mL of anhydrous DCM and 15 g of ground molecular sieves (4 A). The resulting mixture was stirred at room temperature overnight. The reaction mixture was filtered over Celite® and washed with DCM. The filtrate was concentrated under reduced and co-evaporated with toluene (5×10 mL) to yield Intermediate 13 which was used as such without further purification.

Intermediate 14

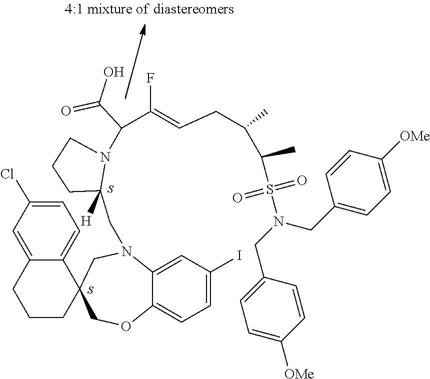

Intermediate 5 (3.9 g, 7.7 mmol), Intermediate 12 (4.6 g, 7.7 mmol, 1 eq.) and glyoxylic acid monohydrate (CAS [563-96-2]) (1.4 g, 15.5 mmol, 2 eq.) were stirred in 150 mL of MeOH at 55° C. during 13 h. After cooling to room temperature, solvents were evaporated under reduced pressure. The crude product was purified by column chromatography eluting with 0-5% MeOH in DCM to afford Intermediate 14 (6.7 g, yield 87%) as an off-white foam as ca 4:1 mixture of diastereomers.

Intermediate 15

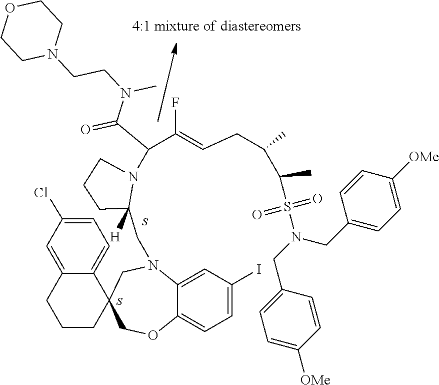

1-propanephosphonic acid anhydride (0.183 mL, 0.31 mmol, 2 eq.) was added dropwise to a stirred solution of Intermediate 14 (150 mg, 0.15 mmol), N-methyl-2-morpholinoethanamine (CAS [41239-40-1]) (100 mg, 0.696 mmol, 4.6 eq.) and triethylamine (0.151 mL, 1.083 mmol, 7.2 eq.) in 7.5 mL of anhydrous DCM. The resulting mixture was stirred at room temperature overnight. The mixture was washed with brine, and the organic layer was dried (MgSO₄), filtered and evaporated under reduced pressure. The crude product was purified on silica gel with heptane/EtOAc (1:0 to 0:1). The purest fractions were evaporated to give Intermediate 15 (168 mg, yield 99%) as a white solid.

Intermediate 16

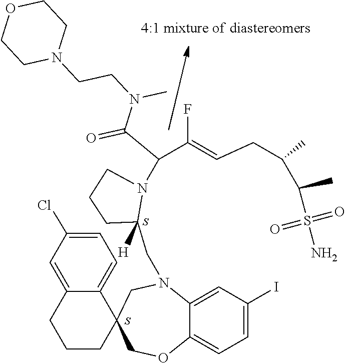

TFA (1.63 mL, 21.3 mmol) was added dropwise to a stirred solution of Intermediate 15 (168 mg, 0.149 mmol) in 1.5 mL of anhydrous DCM at 0° C. The resulting mixture was allowed to warm to room temperature and stirred 18 h. DCM and sat. Sol. NaHCO₃ were added. The aqueous layer was extracted with DCM, the combined organic layers were dried (MgSO₄), filtered and evaporated under reduced pressure to get Intermediate 16 (132 mg, quant.). The crude product was used as such in the next step.

Intermediate 17

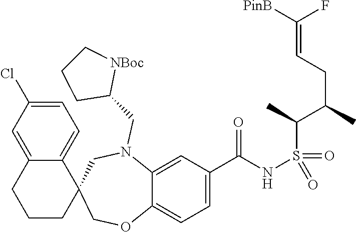

EDCI (4.6 g, 24.1 mmol, 2.5 eq.) was added to a stirred mixture of Intermediate 8 (5.0 g, 9.5 mmol), Intermediate 13 (6.2 g, 15.4 mmol,), DMAP (2.5 g, 20.5 mmol, 2.2 eq.) and triethylamine (10 mL, 71.9 mmol, 7.5 eq.) in 130 mL of anhydrous DCM. The resulting mixture was stirred 24 h at room temperature.

The mixture was diluted with DCM (50 mL) and quenched with 1 M HCl sol. (100 mL). The organic layer was extracted with DCM, and the combined organic layers were washed with brine (100 mL), dried (MgSO₄), filtered and evaporated under reduced pressure. The crude product was purified by silica gel column chromatography with Hept/EtOAc (1:0 to 3:7) to afford Intermediate 17 (6.75 g, yield 86%) as a white solid.

Intermediate 18

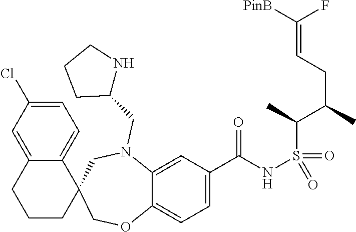

TFA (2.5 mL, 1.49 g/mL, 33 mmol) was added dropwise to a solution of Intermediate 17 (600 mg, 0.48 mmol) in 7.5 mL of anhydrous DCM at room temperature and the resulting mixture was stirred at room temperature for 20 h. Then the reaction mixture was concentrated under reduced pressure and co-evaporated with toluene. The residue was used as such in the next step without further purification as the TFA salt.

Intermediate 19

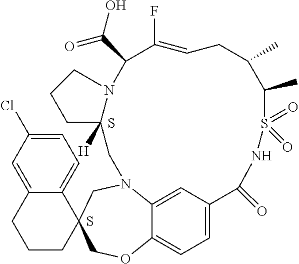

Intermediate 18 (1.0 g, 0.85 mmol), triethylamine (118 μL, 0.85 mmol, 1 eq.) and glyoxylic acid monohydrate (CAS [563-96-2]) (1.0 g, 10.86 mmol, 12 eq.) were stirred in 100 mL of MeOH at reflux during 8 hours. Then, the mixture was evaporated under reduced pressure and used as such as a ca 95/5 diastereomeric mixture in the next step.

Alternatively, the reaction was performed in EtOAc. When full conversion of the starting material is observed, amide coupling can be performed in situ.

Intermediate 20

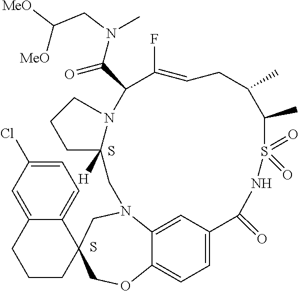

Propanephosphonic acid anhydride (620 μL, 1.08 g/mL, 1.05 mmol) was added to a stirred solution of Intermediate 19 (450 mg, 0.41 mmol), (methylamino)acetaldehyde dimethyl acetal (250 μL, 0.98 g/mL, 2.06 mmol) and triethylamine (570 μL, 0.73 g/mL, 4.11 mmol) in 16 mL of anhydrous DCM. The resulting mixture was stirred at room temperature overnight, then more propanephosphonic acid anhydride (620 μL, 1.08 g/mL, 1.052 mmol) and methylaminoacetaldehyde dimethyl acetal (250 μL, 0.98 g/mL, 2.06 mmol) were added. The mixture was heated at 40° C. and stirred over weekend. Upon cooling to room temperature, the mixture was diluted with DCM and quenched with brine (30 mL). The aqueous layer was extracted with DCM, the combined organic layers were washed with brine (30 mL), dried (MgSO₄), filtered and evaporated under reduced pressure to get Intermediate 20 (322 mg, quant.) which was used without further purification in the next step.

Intermediate 21

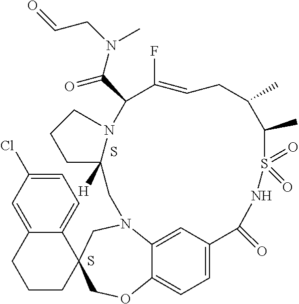

Intermediate 20 (322 mg) was taken up in 35 mL of acetone and 5 mL of water, followed by addition of PTSA (140.851 mg, 0.818 mmol). The resulting mixture was stirred at 50° C. during 18 h. Then, acetone was evaporated under reduced pressure and EtOAc was added. The aqueous layer was extracted with EtOAc, washed with brine (20 mL), dried (MgSO₄), filtered and evaporated under reduced pressure. The crude product was passed through a small pad of silica eluting DCM/MeOH (1:0 then 95:5) to get Intermediate 21 (112 mg, yield 38%) as a yellow solid which was used without further purification for the next step.

Intermediate 22

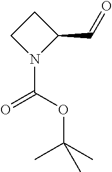

To a stirred solution of (S)-1-Boc-2-azetidinemethanol (CAS [161511-85-9]) (3.60 g, 19.2 mmol) in 30 mL of anhydrous DCM was added trichloroisocyanuric acid (4.47 g, 19.2 mmol). The resulting mixture was cooled to 0° C. prior to portionwise addition of TEMPO (30.0 mg, 0.192 mmol). The ice-bath was removed and the mixture was stirred 30 min at room temperature. EtOAc (50 mL) was added. After 10 min of stirring, the mixture was filtered over celite and washed with EtOAc. The organic layer was washed with sat. Na₂CO₃ sol. (2×50 mL), 0.5 M HCl sol. (30 mL) and brine (3×30 mL), dried (Na₂SO₄), filtered off and evaporated under reduced pressure to afford the crude Intermediate 22 (3.5 g, yield 99%) which was used as such for the next step.

Intermediates 23, 24, 25 and 26

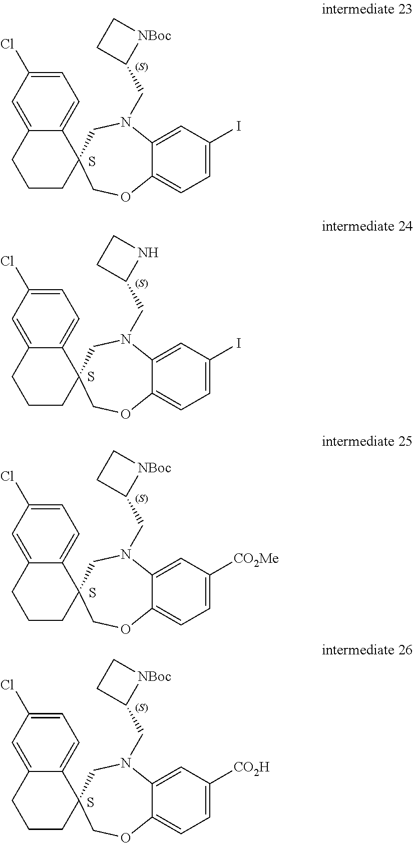

Azetidine analogues (n=1) of Intermediates 4, 5, 7 and 8 were made by using an analogous reaction protocol, starting from Intermediate 22 instead of N-Boc-L-prolinal.

Intermediates 27 and 28

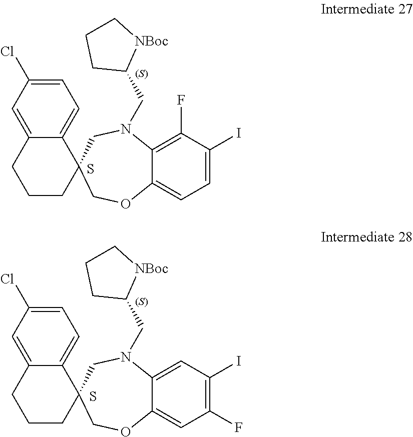

Alternatively to the route presented in Scheme 5,

1-chloromethyl-4-fluoro-1,4-diazoniabicyclo[2.2.2]octane bis(tetrafluoroborate) [CAS 140681-55-6] (1.75 g, 4.93 mmol) was added portionwise to a stirred solution of Intermediate 4 (3 g, 4.93 mmol) in 80 mL of dry acetonitrile at 0° C. The resulting mixture was allowed to warm to room temperature and stirred 16 h.

The reaction mixture was poured into water and extracted with ethyl acetate. The organic layer was washed with water, dried (MgSO4), filtered off and evaporated under reduced pressure.

A purification was performed via Prep HPLC (Stationary phase: RP XBridge Prep C18 OBD-10 μm, 30×150 mm, Mobile phase: 0.25% NH₄HCO₃ solution in water, CH₃CN) followed by Prep SFC (Stationary phase: Chiralpak Daicel AD 20×250 mm, Mobile phase: CO₂, EtOH+0.4 iPrNH₂) to yield Intermediate 27 (115 mg, 4%) and Intermediate 28 (55 mg, 2%) as off-white solids.

Intermediate 29

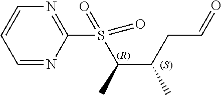

2-(((2R,3S)-3-Methylhex-5-en-2-yl)sulfonyl)pyrimidine (CAS [1638587-10-6]) (2.2 g, 9.154 mmol) was dissolved in DCM (55 mL) and the resulting solution was cooled to −78° C. Ozone (440 mg, 9.154 mmol) was subsequently bubbled through until a grey-blue persistent colour was observed (after 25 min). Nitrogen was then bubbled through the solution (still at −78° C.), this was followed by addition of triphenylphosphine (3400 mg, 12.96 mmol). The reaction mixture was stirred over the weekend at room temperature. The reaction mixture was concentrated under reduced pressure.

The crude product was purified by flash column chromatography on silica gel (heptane:EtOAc-1:0 to 0:1) to get Intermediate 29 (3 g, 66% yield, estimated purity 50% due to presence of triphenylphosphine oxide).

Intermediate 30

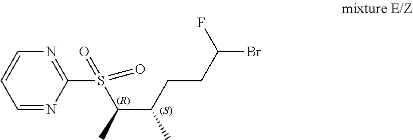

Diethylzinc (1M in heptane) (CAS [557-20-0]) (6.9 mL, 1 M, 6.9 mmol) was added dropwise via syringe pump over 2 h to a stirred solution of Intermediate 29 (1.4 g, 3.467 mmol), fluorotribromomethane (CAS [353-54-8]) (0.7 mL, 2.765 g/mL, 7.149 mmol) and triphenylphosphine (2 g, 7.625 mmol) in anhydrous THE (30 mL).

The solution was quenched 30 min. after addition with 2 mL of MeOH and evaporated under reduced pressure. The crude was subjected to column chromatography with heptane/EtOAc (1:0 to 4:1) to afford Intermediate 30 (500 mg, yield 43%) as an E/Z mixture.

Intermediate 31

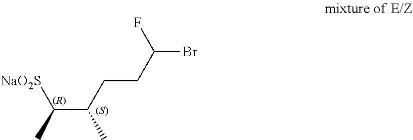

Intermediate 30 (500 mg, 1.483 mmol) was dissolved in methanol (3.5 mL), and to this solution NaOMe (30% in MeOH) (0.275 mL, 1.483 mmol) was added dropwise at 0° C., while stirring. The mixture was stirred for 15 min at 0° C. and then for 30 min at room temperature. The solvent was removed by evaporation. The residue was dissolved in water (10 mL), the side product was extracted with EtOAc. The water layer was used as such in the next step without further purification.

Intermediate 32

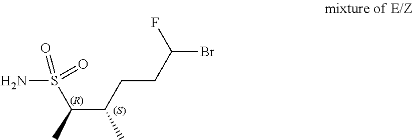

Intermediate 31 in water was stirred at 5° C. A solution of sodium acetate (170 mg, 2.072 mmol) and hydroxylamine-O-sulfonic acid (235 mg, 2.078 mmol) in water (10 mL) was added to the RM whilst keeping the temperature below 10° C. This suspension was stirred at 20° C. for 16 h. The pH of the RM was adjusted to pH=6 with NaHCO₃ solid whilst keeping the temperature below 10° C. The pH-meter of the Opti-max apparatus (Mettler-Toledo) was used for this. The product was extracted with 3×15 mL MTBE. The combined OL's were washed with brine and then dried with Na₂SO₄, filtered and concentrated to get Intermediate 32 (340 mg, 83%).

Form Intermediate 32, Intermediate 10 can be obtained by PMB protection following the reported procedures.

Intermediate 33

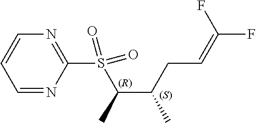

Sodium chlorodifluoroacetate (2000 mg, 13.118 mmol) was added to a stirred solution of Intermediate 29 (49% pure) (3000 mg, 6.067 mmol) and triphenylphosphine (3800 mg, 14.488 mmol) in 60 mL of anhydrous DMF. The resulting mixture was then heated to 100° C. for 3 h. More sodium chlorodifluoroacetate (1000 mg, 6.559 mmol) was added at 100° C., and the reaction mixture was stirred for 2 more h at 100° C. After cooling to 0° C., water was carefully added. The mixture was extracted with Et₂O (2×100 mL), the combined organic layers were washed with water (50 mL) and brine (50 mL), then dried (MgSO₄), filtered off and evaporated under reduced pressure. The crude was purified by column chromatography on silica gel with Hept/EtOAc (100:0 to 70:30) to afford Intermediate 33 (700 mg, yield 41%).

Intermediate 34

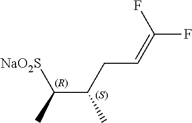

Intermediate 33 (700 mg, 2.533 mmol) was dissolved in methanol (6 mL) and to this solution NaOMe (30% in MeOH) (0.47 mL, 2.538 mmol) was added dropwise at 0° C., while stirring. The reaction mixture was stirred for 15 min at 0° C. and then for 30 min at 20° C. The solvent was removed by evaporation. The residue was dissolved in water (15 mL), the side product was washed away with EtOAc (2×10 mL) and the water layer was used as such in the next step without further purification.

Intermediate 35

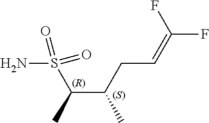

Intermediate 35 in water was stirred at 5° C. A solution of sodium acetate (300 mg, 3.657 mmol) and hydroxylamine-O-sulfonic acid (410 mg, 3.625 mmol) in water (15 mL) was added to the reaction mixture, while keeping the temperature below 10° C. This reaction mixture was stirred at 20° C. for 16 h. The pH of the mixture was adjusted to pH=6 with NaHCO₃ solid while keeping the temperature below 10° C. MTBE was added. The organic layer was separated and the aqueous layer was back-extracted with MTBE (×3). The combined organic layers were washed with brine and then dried with Na₂SO₄, filtered and concentrated to yield Intermediate 35 (465 mg, 86%).

Intermediate 36

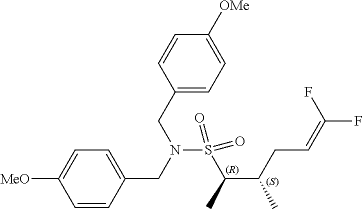

To a stirred solution of Intermediate 35 (460 mg, 2.157 mmol) in DMF (4 mL) was added potassium carbonate (1.2 g, 8.683 mmol) followed by dropwise addition of 4-methoxybenzyl chloride (0.9 mL, 1.155 g/mL, 6.637 mmol). The resulting suspension was stirred at 70° C. during 18 h. After cooling to room temperature, solids were filtered off through a pad of celite and washed with EtOAc. The filtrate was evaporated under reduced pressure to remove most of the DMF. Then, the yellow residue was taken up in 100 mL of EtOAc and washed with brine (2×10 mL). The organic layer was dried (MgSO₄), filtered off and evaporated under reduced pressure. The residue was purified by flash column chromatography on silica gel (40 g Redisep flash column eluting with 0-40% EtOAc in heptane) to afford Intermediate 36 (870 mg, 80%) as a clear oil.

From Intermediate 36, Intermediate 12 can be obtained according to the following procedure:

Copper(I) chloride (1.096 mg, 0.0111 mmol) was placed in a2 mL crimped vial equipped with a stir bar together with 4,5-bis(diphenylphosphino)-9,9-dimethylxanthene (6.404 mg, 0.0111 mmol). The vial was sealed and purged (N₂, 3 exchanges), then dried and degassed. 1,2-dimethoxyethane (1.107 mL, 0.1 M, 0.111 mmol) was added and the reaction mixture was heated to 40° C. and aged for 1 h at this temperature. Intermediate 36 (50.2 mg, 0.111 mmol) was placed in a 40 mL vial equipped with a stir bar then bis(pinacolato)diboron (67.457 mg, 0.266 mmol) was added. Vial was sealed and purged (N₂, 3 exchanges), then the cooled (rt) CuCl/Xantphos/DME mixture was added under inert atmosphere to the substrates vial. The crimped vial was rinsed (2×50 μL DME) and the rinsates were added to the main reaction vial. Dried and degassed MeOH (9.0 μL, 0.791 g/mL, 0.221 mmol) was added in a single addition to the reaction mixture then tBuOK (221 μL, 1 M, 0.221 mmol) was added dropwise over 10 minutes at rt. The reaction was aged at rt for 2 h, then was heated to 40° C. and aged at this temperature. After 16 h, the reaction was cooled to rt then a saturated aqueous NaHCO₃ solution (10 mL) followed by AcOEt (10 mL) were added to the mixture. The crude mixture was poured into a separatory funnel and phases were separated. Aqueous phase was extracted 3 times with AcOEt (10 mL), organics were combined, washed (brine), dried (MgSO₄) then solvents removed under reduced pressure. Crude material was purified over silica gel eluting heptane/AcOEt and to afford Intermediate 12 (31.2 mg, 48%) as a colorless oil.

Intermediate 37

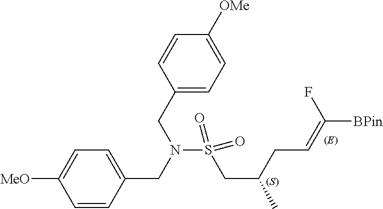

Intermediate 49 was prepared in an analogous manner as Intermediate 12, following the route from 9 to 12, starting from the known intermediate (S)-N,N-bis(4-methoxybenzyl)-2-methylpent-4-ene-1-sulfonamide (CAS [1883727-89-6]) Intermediate 38

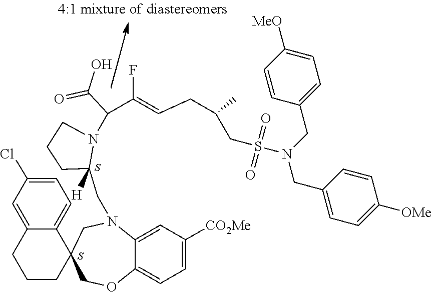

Intermediate 38 was prepared by analogy with Intermediate 14, using Intermediate 37 as Petasis coupling partner.

Intermediate 39

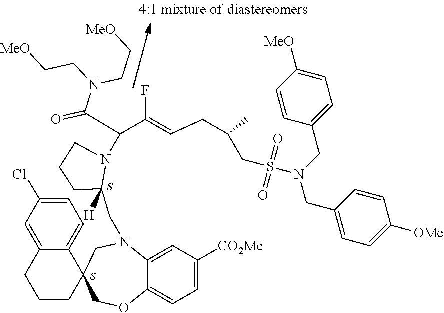

Intermediate 39 was prepared by analogy with Intermediate 15.

Intermediate 40

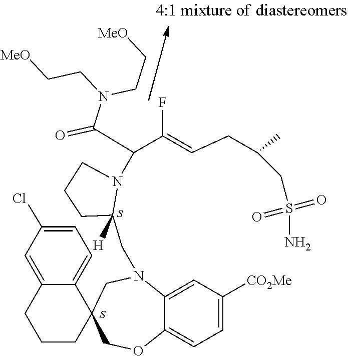

Intermediate 40 was prepared by analogy with Intermediate 15.

Intermediate 41

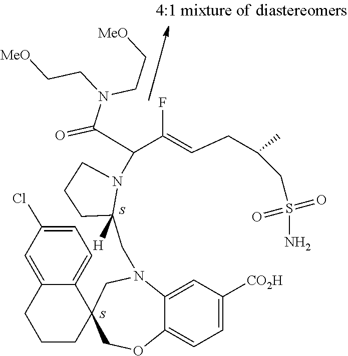

LiOH (15 mg, 0.626 mmol) was added to a stirred mixture of Intermediate 40 (100 mg, 0.126 mmol) in 3 mL of THF, 3 mL of water and 3 mL of MeOH. The resulting mixture was stirred at room temperature for 36 h. Upon completion, the mixture was diluted with DCM and quenched with 1 M HCl sol. The organic layer was separated and the aqueous layer was back-extracted with DCM (×3). The combined dried (MgSO₄) organic layers were evaporated under reduced pressure to give Intermediate 41 (100 mg, quant.) as a yellow froth, which was used without further purification for the next step.

Intermediate 42

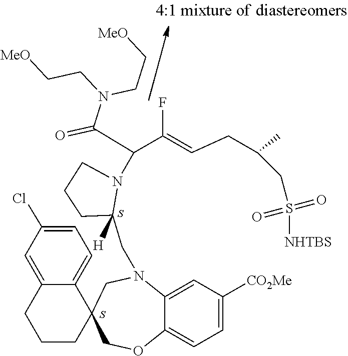

A solution of Intermediate 40 (1.5 g, 1.891 mmol) in 70 mL of anhydrous THE was cooled to 0° C., followed by addition of triethylamine (0.526 mL, 0.728 g/mL, 3.781 mmol) and then dropwise addition of a solution of tert-butyldimethylsilyl chloride (CAS 5 [18162-48-6], 370 mg, 2.458 mmol) in 10 mL of anhydrous THF. The resulting mixture was allowed to warm to room temperature and stirred 3 d, then heated at 50° C. for 7 d.

The precipitate was filtered over celite and washed with Et₂O. The filtrate was evaporated under reduced pressure and the crude purified by column chromatography with Hept/EtOAc (1:0 to 4:1 then to 0:1) to afford Intermediate 42 (750 mg, yield 44%) as an off-white froth and recover some of Intermediate 40 (622 mg, yield 42%) as a yellow oil.

Intermediate 43

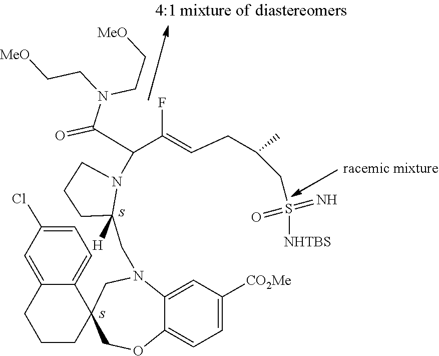

To a stirred suspension of dichlorotriphenylphosphorane (CAS [2526-64-9], 450 mg, 1.351 mmol) in 15 mL of anhydrous DCM was added triethylamine (0.4 mL, 0.728 g/mL, 2.728 mmol). After 10 min, the mixture was cooled to 0° C., followed by addition of a solution of Intermediate 42 (0.65 g, 0.716 mmol) in 3 mL of anhydrous DCM. After 1 h at 0° C., ammonia was bubbled for 2 min to the reaction mixture, which was stirred 2 h at 0° C. After filtration of the precipitate on dicalite, the filtrate was evaporated under reduced pressure. The crude was purified by FCC with Hept/EtOAc (100:0 to 0:100). The product fractions were collected and concentrated to give Intermediate 43 (700 mg, 77%) as a yellow oil.

Intermediate 44

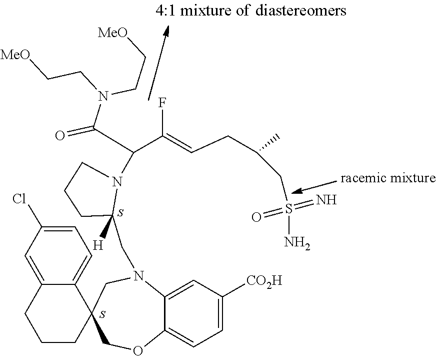

Intermediate 43 (600 mg, 0.476 mmol) was dissolved in 7 mL of THF, then 7 mL of water was added followed by LiOH (98 mg, 4.092 mmol). The resulting mixture was stirred for 72 h at RT.

This reaction mixture was acidified with HCl 1 N until pH=2. This was stirred for 15 min. The organic layer was separated and the aqueous layer was treated with NaHCO₃ sat. solution until pH=5, then a new extraction was performed with DCM. The organic layer was dried (MgSO4), filtered and evaporated to give Intermediate 44 (250 mg, 67%) which was used as such without further purification.

Intermediate 45

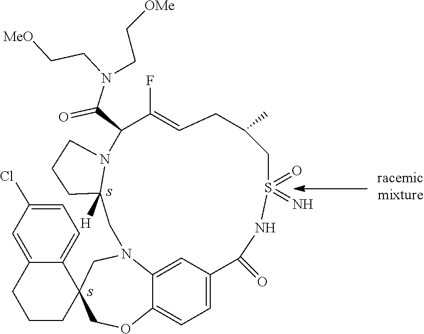

To a stirred solution of Intermediate 44 (250 mg, 0.321 mmol) in 20 mL of acetonitrile (20 mL) was added pyridazine (600 mg, 7.492 mmol), followed by thionyl chloride (100 μL, 1.378 mmol). The reaction mixture was stirred for 4 hours at 30° C. The mixture was evaporated and taken up in DCM and water, then HCl 1N was added until pH=5. The organic layer was separated, dried (MgSO₄), filtered off and concentrated to yield Intermediate 45 (245 mg, quant.) which was used as such without further purification.

Intermediate 46

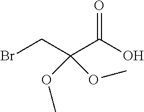

Bromopyruvic acid (10 g, 59.895 mmol) was dissolved in trimethyl orthoformate (20 mL, 182.809 mmol) and sulfuric acid (0.86 mL, 16.134 mmol) under N₂. The reaction mixture was stirred at RT for 20 hours. The reaction mixture was quenched with 10% aqueous hydrochloric acid. This mixture was extracted with DCM (3×100 mL). The combined organic layers were dried over sodium sulfate, filtered and concentrated in vacuo to obtain Intermediate 46 (6000 mg, 47%).

Intermediate 47

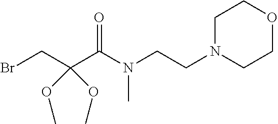 [SMILES: COC(CBr)(OC)C(=O)N(C)CCN1CCOCC1]

N-methyl-2-morpholinoethanamine (4560 mg, 31.619 mmol), DIPEA (13 mL, 75.438 mmol) and HBTU (13 g, 34.279 mmol) were added to a solution of Intermediate 46 (5550 mg, 26.053 mmol) in DCM (130 mL). The reaction mixture was stirred overnight at room temperature. The reaction mixture was washed with HCl 3 M (2×100 mL), then the organic layer was dried over MgSO₄, filtered and concentrated in vacuo. The residue was purified by FCC using a 120 g redisep flashcolumn and DCM/Methanol(NH₃) going from 100:0 to 93:7 as eluent. The product fractions were collected and concentrated. To get Intermediate 47 (7460 mg, 84%).

Intermediate 48

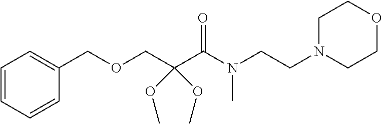 [SMILES: COC(COCc1ccccc1)(OC)C(=O)N(C)CCN1CCOCC1]

Benzyl alcohol (1950 mg, 18.032 mmol) was added to a flask containing NaH (60% dispersion in mineral oil) (1500 mg, 37.504 mmol) in Me-THF (60 mL) at 0° C. under N2 atmosphere. The reaction mixture was stirred at Tⱼ=70° C. for 1 hour. Intermediate 47 (3000 mg, 8.844 mmol) in Me-THF (30 mL) was added to the mixture. The reaction mixture was stirred at Tⱼ=70° C. for another 15 hours. The reaction temperature was lowered to room temperature and the reaction mixture was quenched with water (15 mL). 25 mL water was added extra, then the organic layer was separated. The aqueous layer was extracted with DCM (2×50 mL). The combined organic layers were dried over MgSO₄, filtered and concentrated in vacuo. The residue was purified by FCC using a 120 g redisep flashcolumn and DCM/Methanol(NH₃) going from 100:0 to 93:7 as eluent.

The product fractions were collected and concentrated to yield Intermediate 48 (1350 mg, 41%)

Intermediate 49

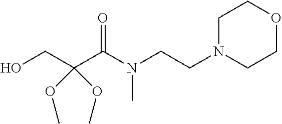 [SMILES: COC(CO)(OC)C(=O)N(C)CCN1CCOCC1]

Intermediate 48 (700 mg, 1.91 mmol) was dissolved in Methanol (40 mL) in a hydrogenation flask, Pd/C (10%) (200 mg) was added. The reaction flask was degassed and flushed with hydrogen and then hydrogenated at 50° C. over 2 nights. After cooling, the solids were filtered over dicalite and the filtrate was concentrated to dryness to get Intermediate 49 (530 mg, 81%) which was used in the next step without further purification.

Intermediate 50

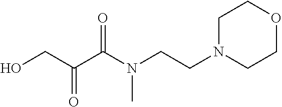 [SMILES: CN(CCN1CCOCC1)C(=O)C(=O)CO]

Intermediate 49 (530 mg, 1.554 mmol) was dissolved in 1,4-dioxane (6 mL), then HCl (6 M in water) (3 mL, 18 mmol) was added, the reaction vial was closed and the RM was stirred at 60° C. for 16 h. After cooling the solvents were concentrated until a dry residue was obtained as an HCl salt. This crude residue Intermediate 50 (414 mg, quant.) was used in the next step without further purification.

Preparation of Compounds

Compound 1

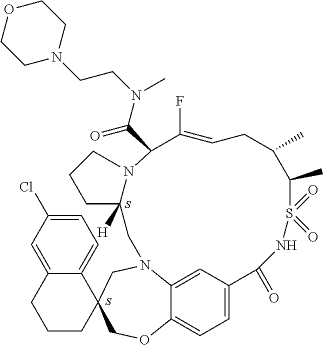

Intermediate 16 (132 mg, 0.149 mmol) was dissolved in 30 mL of anhydrous THF, followed by addition of DBU (135 μL, 0.905 mmol, 6 eq.). The mixture was purged and degassed with nitrogen, followed by addition of Pd(dppf)Cl₂ (CAS [72287-26-4]) (17 mg, 0.0238 mmol, 15 mol %). Then, the RM was purged with CO and the mixture was heated at 100° C. under ca 30 bars CO pressure during 7 h. After cooling to room temperature, the reaction mixture was concentrated, the residue was taken up in DCM and water, then just enough acetic acid was added to neutralize the DBU. The layers were separated, and the water layer was extracted once more with DCM. The combined organic layers were dried (MgSO₄), filtered and evaporated under reduced pressure. The residue was subjected to column chromatography eluting with 0-5% MeOH in DCM to afford the crude, impure product which then was purified by preparative SFC (Stationary phase: Chiralpak Diacel AD 20×250 mm, Mobile phase: CO₂, EtOH+0.4 iPrNH₂) to yield Compound 1 (26 mg, 22%) as a white solid.

¹H NMR δ 1.06 (d, J=6.38 Hz, 3H) 1.36-1.45 (m, 4H) 1.63-1.74 (m, 3H) 1.75-1.89 (m, 1H) 1.89-2.07 (m, 5H) 2.31-2.41 (m, 1H) 2.46-2.60 (m, 5H) 2.64-2.84 (m, 3H) 2.90 (br dd, J=15.00, 10.19 Hz, 1H) 3.00 (s, 2H) 3.06-3.24 (m, 2H) 3.25-3.34 (m, 3H) 3.34-3.51 (m, 2H) 3.59-3.84 (m, 5H) 3.90-4.03 (m, 2H) 4.07-4.21 (m, 2H) 4.45-4.64 (m, 1H) 4.74-5.00 (m, 1H) 6.86-7.04 (m, 3H) 7.09 (d, J=2.30 Hz, 1H) 7.21 (d, J=7.91 Hz, 1H) 7.69 (d, J=8.47 Hz, 1H); LCMS confirms the MW (Rt: 2.06, MW: 785.30, [M+H]⁺ 786, Method 3); SFC (Rt: 4.37, 0.00% isomer 1), (Rₜ: 6.73, 100.00% isomer 2) (Rₜ: 6.73, Area %: 100.00, MW: 785.34, [M+H]⁺ 786, Method 11).

The following Compounds were made by analogy with Compound 1, following the pathway described in Scheme 1 and starting from the respective starting materials.

TABLE 2

| Compound | Formula | Analytical data |
|---|---|---|
| 2 | 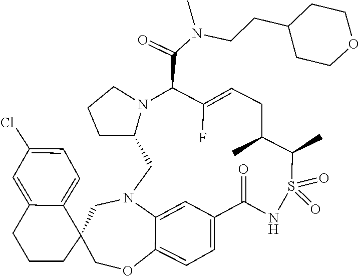 [SMILES: C[C@@H]1[C@@H](C)C/C=C(\F)[C@H](C(=O)N(C)CCC2CCOCC2)N2CCC[C@H]2CN2C[C@@]3(CCCc4cc(Cl)ccc43)COc3ccc(cc32)C(=O)NS1(=O)=O] | ¹H NMR (400 MHz, DMSO-d₆) δ ppm 0.96- 1.01 (m, 3 H) 1.15-1.28 (m, 2 H) 1.28-1.32 (m, 3 H) 1.42-1.54 (m, 3 H) 1.55-1.73 (m, 6 H) 1.78-2.06 (m, 6 H) 2.16-2.26 (m, 1 H) 2.66-2.86 (m, 4 H) 3.08-3.21 (m, 3 H) 3.22- 3.35 (m, 5 H) 3.36-3.54 (m, 2 H) 3.78-3.84 (m, 2 H) 3.86-3.97 (m, 3 H) 3.98-4.09 (m, 1 H) 4.16-4.24 (m, 1 H) 4.49-4.71 (m, 1 H) 4.72-4.94 (m, 1 H) 6.87-6.92 (m, 1 H) 7.00- 7.08 (m, 2 H) 7.12-7.16 (m, 1 H) 7.21-7.26 (m, 1 H) 7.61-7.69 (m, 1 H) 11.13-12.06 (m, 1 H); LCMS confirms the MW (Rt: 1.16, MW: 784.00, [M + H]⁺ 785, Method 1); SFC (Rₜ: 4.65, 100.00% isomer 1) (Area %: 100.00, Method 12). |
| 3 | 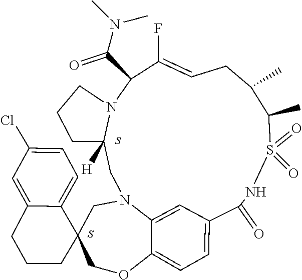 | ¹H NMR δ 1.06 (d, J = 6.27 Hz, 3 H) 1.43 (d, J = 7.21 Hz, 3 H) 1.63-1.85 (m, 6 H) 1.89-2.07 (m, 5 H) 2.36 (br dd, J = 14.32, 8.67 Hz, 1 H) 2.69-2.83 (m, 2 H) 2.87-2.98 (m, 2 H) 3.00 (s, 3 H) 3.12-3.20 (m, 1 H) 3.26 (s, 3 H) 3.34 (br d, J = 14.21 Hz, 2 H) 3.89-4.02 (m, 3 H) 4.13-4.24 (m, 2 H) 4.46- 4.62 (m, 1 H) 4.74-4.98 (m, 1 H) 6.85-6.91 (m, 1 H) 6.91-6.98 (m, 1 H) 7.01 (s, 1 H) 7.09 (d, J = 2.19 Hz, 1 H) 7.21 (d, J = 8.70 Hz, 1 H) 7.69 (d, J = 8.47 Hz, 1 H); LCMS confirms the MW (Rₜ: 1.13, MW: 686.00, [M + H]⁺ 687, Method 1). |
| 4 | 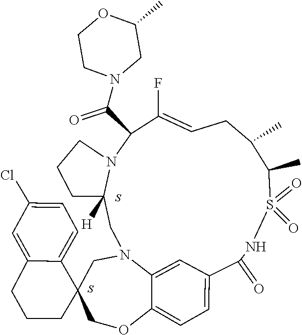 | ¹H NMR δ 1.21 (d, J = 6.16 Hz, 3 H) 1.28- 1.37 (m, 3 H) 1.43 (dd, J = 7.26, 1.98 Hz, 3 H) 1.61-1.84 (m, 4 H) 1.88-2.09 (m, 5H) 2.18-2.58 (m, 2 H) 2.71-2.88 (m, 3 H) 2.91- 3.07 (m, 2 H) 3.13-3.36 (m, 4 H) 3.45-3.62 (m, 2 H) 3.86-4.02 (m, 4 H) 4.06-4.22 (m, 3 H) 4.36-4.44 (m, 1 H) 4.44-4.68 (m, 1 H) 4.81 (br s, 1 H) 6.86-6.92 (m, 1 H) 6.93- 6.97 (m, 1 H) 6.97-7.04 (m, 1 H) 7.07-7.11 (m, 1 H) 7.20 (d, J = 8.65 Hz, 1 H) 7.68 (d, J = 8.58 Hz, 1 H) 8.10 (br s, 1 H); LCMS confirms the MW (Rₜ: 2.41, MW: 742.00, [M + H]⁺ 743, Method 4). |
| 5 | 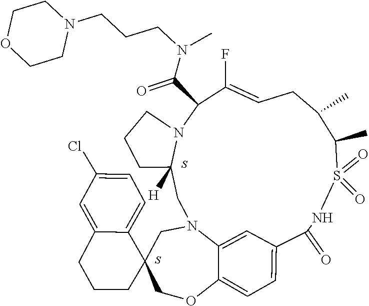 | ¹H NMR δ 1.01-1.09 (m, 3 H) 1.16-1.21 (m, 3 H) 1.35-1.41 (m, 4 H) 1.67-1.74 (m, 3 H) 1.77-1.93 (m, 3 H) 1.99-2.08 (m, 3 H) 2.16- 2.33 (m, 2 H) 2.34-2.50 (m, 6 H) 2.73-2.81 (m, 2 H) 2.93-2.99 (m, 3 H) 3.09-3.17 (m, 2 H) 3.31-3.38 (m, 2 H) 3.44-3.56 (m, 2H) 3.63-3.67 (m, 1 H) 3.69-3.74 (m, 3 H) 3.92- 4.03 (m, 3 H) 4.14-4.20 (m, 1 H) 4.45-4.69 (m, 1 H) 4.90-5.08 (m, 1 H) 5.17-5.51 (m, 1 H) 6.87-6.92 (m, 1 H) 6.98-7.06 (m, 1 H) 7.06-7.10 (m, 2 H) 7.17-7.22 (m, 1 H) 7.69-7.73 (m, 1 H); LCMS confirms the MW (Rₜ: 1.98, MW: 799.40, [M + H]⁺ 800, Method 6). |
| 6 | 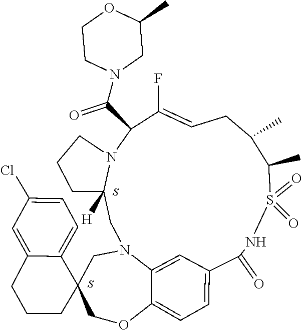 | LCMS confirms the MW (Rₜ: 2.49, MW: 742.00, [M + H]⁺ 743, Method 3). |
| 7 | 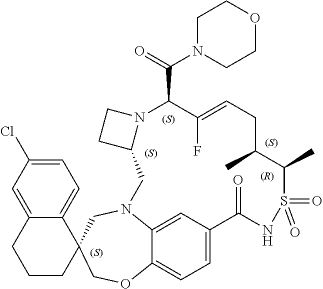 | ¹H NMR δ 1.05-1.09 (m, 3 H) 1.41-1.47 (m, 4 H) 1.75-1.86 (m, 1 H) 1.91-1.97 (m, 1 H) 1.98-2.08 (m, 4 H) 2.14-2.24 (m, 1 H) 2.33- 2.44 (m, 1 H) 2.69-2.84 (m, 2 H) 3.24 (s, 2 H) 3.45-3.54 (m, 1 H) 3.56-3.64 (m, 3 H) 3.66-3.85 (m, 9 H) 4.03-4.18 (m, 3 H) 4.24- 4.37 (m, 1 H) 4.75-4.92 (m, 1 H) 6.93-7.06 (m, 3 H) 7.09 (d, J = 2.2 Hz, 1 H) 7.15-7.21 (m, 1 H) 7.67 (d, J = 8.6 Hz, 1 H); LCMS confirms the MW (Rₜ: 2.42, MW: 714.00, [M + H]⁺ 715, Method 3). |
| 8 | 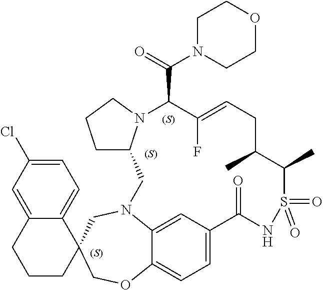 | ¹H NMR δ 1.03-1.10 (m, 3 H) 1.44 (d, J = 7.3 Hz, 4 H) 1.64-1.76 (m, 3 H) 1.76-1.86 (m, 1 H) 1.91-2.08 (m, 5 H) 2.30-2.41 (m, 1 H) 2.72 (br d, J = 5.3 Hz, 2 H) 2.84-2.92 (m, 1 H) 2.98-3.07 (m, 1 H) 3.08-3.16 (m, 1 H) 3.27-3.39 (m, 2 H) 3.60-3.80 (m, 7 H) 3.82-3.93 (m, 2 H) 3.95-4.03 (m, 2 H) 4.10- 4.17 (m, 1 H) 4.17-4.23 (m, 1 H) 4.44-4.60 (m, 1 H) 4.77-4.96 (m, 1 H) 6.88-6.93 (m, 1 H) 6.94-6.98 (m, 1 H) 6.98-7.03 (m, 1 H) 7.08-7.12 (m, 1 H) 7.18-7.24 (m, 1 H) 7.65- 7.74 (m, 1 H) 7.84-8.47 (m, 1 H); LCMS confirms the MW (Rₜ: 1.11, MW: 728.00, [M + H]⁺ 729, Method 1). |
| 9 | 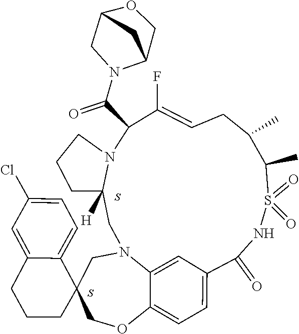 | ¹H NMR δ 1.03-1.07 (m, 3 H) 1.37-1.47 (m, 4 H) 1.59-1.73 (m, 5 H) 1.77-1.83 (m, 1 H) 1.89-1.95 (m, 2 H) 1.96-2.06 (m, 4 H) 2.27- 2.41 (m, 1 H) 2.78 (br s, 4 H) 3.10-3.21 (m, 1 H) 3.25-3.38 (m, 2 H) 3.80-3.86 (m, 1 H) 3.88-4.01 (m, 4 H) 4.13-4.22 (m, 2 H) 4.24- 4.53 (m, 1 H) 4.65-5.01 (m, 2 H) 4.66-4.69 (m, 1 H) 4.71-4.77 (m, 1 H) 4.81-4.97 (m, 1 H) 6.88-7.03 (m, 3 H) 7.07-7.11 (m, 1 H) 7.17-7.23 (m, 1 H) 7.69 (dd, J = 8.5, 2.2 Hz, 1 H); LCMS confirms the MW (Rₜ: 1.98, MW: 740.00, [M + H]⁺ 741, Method 4). |
| 10 | 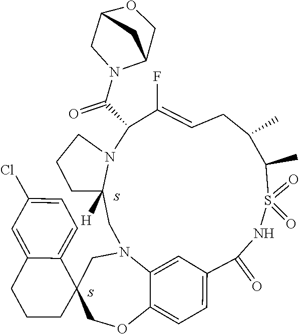 | ¹H NMR δ 1.08-1.15 (m, 3 H) 1.40-1.46 (m, 4 H) 1.54-1.64 (m, 5 H) 1.73-1.85 (m, 5 H) 1.89-1.98 (m, 3 H) 2.04-2.20 (m, 2 H) 2.69- 2.88 (m, 3 H) 3.06-3.41 (m, 4 H) 3.51-3.59 (m, 1 H) 3.72-3.88 (m, 4 H) 3.91-4.02 (m, 3 H) 4.08-4.28 (m, 2 H) 4.61-4.68 (m, 1 H) 4.95-5.11 (m, 1 H) 6.90-6.95 (m, 1 H) 7.08 (br d, J = 2.3 Hz, 2 H) 7.16-7.23 (m, 1 H) 7.59-7.81 (m, 2 H) 7.64-7.74 (m, 2 H); LCMS confirms the MW (Rₜ: 1.98, MW: 740.00, [M + H]⁺ 741, Method 4). |
| 11 | 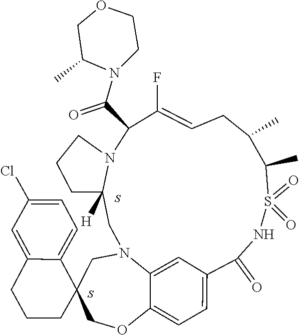 | LCMS confirms the MW (Rₜ: 2.13, MW: 742.3, [M + H]⁺ 743, Method 3); SFC (Rₜ: 3.12, 0.00% isomer 1), (Rₜ: 4.01, 99.08% isomer 2) (Rₜ: 4.01, Area %: 99.08, MW: 742.30, [M + H]⁺ 743, Method 13). |
| 12 | 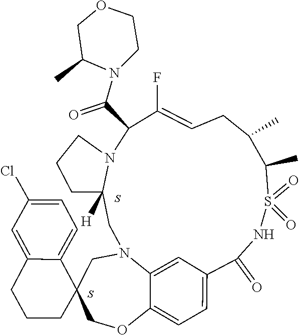 | ¹H NMR δ 1.00-1.08 (m, 3 H) 1.27-1.36 (m, 2 H) 1.40-1.45 (m, 3 H) 1.46-1.52 (m, 1 H) 1.62-1.86 (m, 4 H) 1.91-1.99 (m, 2 H) 2.03 (br d, J = 9.5 Hz, 2 H) 2.22-2.37 (m, 1 H) 2.68-2.81 (m, 2 H) 2.82-2.98 (m, 1 H) 3.04- 3.20 (m, 2 H) 3.29-3.38 (m, 2 H) 3.41-3.54 (m, 2 H) 3.55-3.65 (m, 1 H) 3.69-3.82 (m, 1 H) 3.85-4.01 (m, 4 H) 4.02-4.27 (m, 3 H) 4.29-4.49 (m, 1 H) 4.52-4.69 (m, 1 H) 4.78- 4.95 (m, 1 H) 6.87-6.97 (m, 2 H) 6.97-7.05 (m, 1 H) 6.99-7.15 (m, 2 H) 7.07-7.12 (m, 1 H) 7.16-7.23 (m, 1 H) 7.68 (d, J = 8.4 Hz, 1 H); LCMS confirms the MW (Rₜ: 2.11, MW: 742.00, [M + H]⁺ 743, Method 4). |
| 13 | 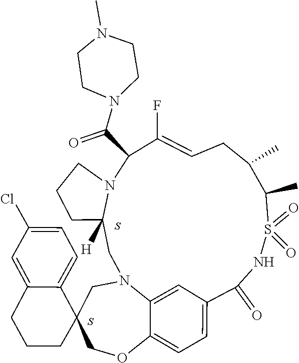 | ¹H NMR δ 1.06 (d, J = 6.2 Hz, 3 H) 1.43 (d, J = 7.2 Hz, 4 H) 1.64-1.74 (m, 3 H) 1.75- 1.87 (m, 1 H) 1.92-2.05 (m, 5 H) 2.36 (s, 4 H) 2.42-2.57 (m, 4 H) 2.71-2.84 (m, 2 H) 2.85-2.94 (m, 1 H) 2.98-3.13 (m, 2 H) 3.28- 3.37 (m, 2 H) 3.68-3.78 (m, 3 H) 3.81-3.90 (m, 2 H) 3.91-4.02 (m, 3 H) 4.11 (q, J = 7.0 Hz, 1 H) 4.19 (d, J = 12.2 Hz, 1 H) 4.47- 4.60 (m, 1 H) 4.77-4.95 (m, 1 H) 6.90-6.97 (m, 2 H) 6.99-7.04 (m, 1 H) 7.10 (d, J = 2.2 Hz, 1 H) 7.21 (dd, J = 8.5, 2.3 Hz, 1 H) 7.70 (d, J = 8.5 Hz, 1 H); LCMS confirms the MW (Rₜ: 1.12, MW: 741.00, [M + H]⁺ 742, Method 2). |
| 14 | 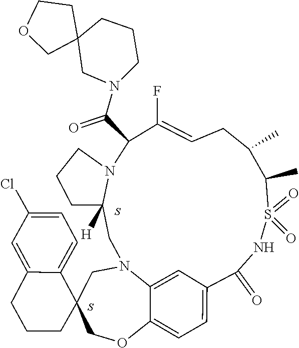 | ¹H NMR δ 1.05 (d, J = 6.0 Hz, 3 H) 1.43 (br d, J = 7.2 Hz, 4 H) 1.57-1.72 (m, 7 H) 1.83 (br d, J = 4.2 Hz, 3 H) 1.90-2.09 (m, 5 H) 2.24-2.41 (m, 1 H) 2.70-2.83 (m, 2 H) 2.89 (dt, J = 14.8, 10.8 Hz, 1 H) 2.96-3.17 (m, 2 H) 3.24-3.39 (m, 2 H) 3.47-3.83 (m, 6 H) 3.87-4.04 (m, 5 H) 4.09-4.23 (m, 2 H) 4.49- 4.63 (m, 1 H) 4.70-4.96 (m, 1 H) 6.89-6.97 (m, 2 H) 7.01 (br s, 1 H) 7.10 (d, J = 2.1 Hz, 1 H) 7.21 (dd, J = 8.4, 2.1 Hz, 1 H) 7.69 (d, J = 8.6 Hz, 1 H) 7.77-9.23 (m, 1 H); LCMS confirms the MW (Rₜ: 1.13, MW: 782.00, [M + H]⁺ 783, Method 1); SFC (Rₜ: 3.50, 0.00% isomer 1), (Rₜ: 4.19, 100.00% isomer 2) (Rₜ: 4.19, Area %: 100.00, Method 14). |
| 15 | 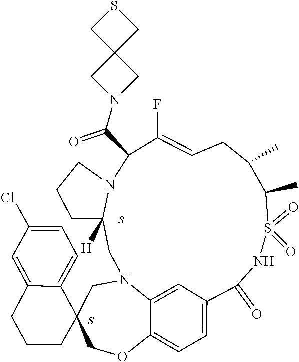 | ¹H NMR δ 1.05 (d, J = 6.5 Hz, 3 H) 1.39- 1.47 (m, 4 H) 1.66 (tt, J = 12.7, 6.5 Hz, 3 H) 1.75-1.94 (m, 3 H) 1.95-2.12 (m, 5 H) 2.29- 2.38 (m, 1 H) 2.71-2.81 (m, 2 H) 2.84-2.93 (m, 2 H) 3.08-3.15 (m, 1 H) 3.26-3.36 (m, 2 H) 3.39-3.43 (m, 3 H) 3.88-4.02 (m, 3 H) 4.04-4.12 (m, 2 H) 4.13-4.21 (m, 2 H) 4.28- 4.36 (m, 1 H) 4.38-4.44 (m, 1 H) 4.80-4.96 (m, 1 H) 6.88-6.96 (m, 2 H) 6.96-6.99 (m, 1 H) 7.08-7.11 (m, 1 H) 7.18-7.23 (m, 1 H) 7.64-7.76 (m, 1 H); LCMS confirms the MW (Rₜ: 2.18, MW: 756.00, [M + H]⁺ 757, Method 3). |
| 16 | 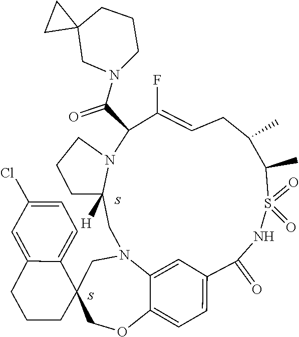 | ¹H NMR δ 0.38 (br d, J = 2.1 Hz, 4 H) 1.05 (d, J = 6.7 Hz, 2 H) 1.16-1.23 (m, 5 H) 1.31- 1.36 (m, 4 H) 1.45-1.58 (m, 1 H) 1.68-1.76 (m, 3 H) 1.95-2.04 (m, 2 H) 2.05-2.15 (m, 1 H) 2.18-2.29 (m, 2 H) 2.69-2.81 (m, 2 H) 2.93 (dd, J = 14.8, 10.1 Hz, 1 H) 3.09 (s, 2 H) 3.28-3.40 (m, 2 H) 3.52-3.63 (m, 3 H) 3.62-3.81 (m, 3 H) 3.88-4.02 (m, 3 H) 4.15 (d, J = 12.2 Hz, 1 H) 4.58 (d, J = 19.6 Hz, 1 H) 5.08 (dt, J = 38.1, 6.8 Hz, 1 H) 6.85 (d, J = 8.1 Hz, 1 H) 7.08 (d, J = 2.3 Hz, 1 H) 7.14-7.22 (m, 3 H) 7.72 (d, J = 8.5 Hz, 1 H); LCMS confirms the MW (Rₜ: 2.35, MW: 752.20, [M + H]⁺ 753, Method 3); SFC (Rₜ: 3.21, 0.00% isomer 1), (Rₜ: 3.99, 100.00% isomer 2) (Rₜ: 3.99, Area %: 100.00, MW: 752.32, [M + H]⁺, Method 13). |
| 17 | 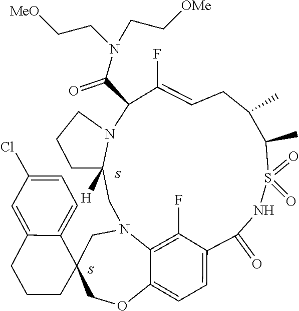 | ¹H NMR δ 1.06 (br d, J = 6.9 Hz, 3 H) 1.20- 1.35 (m, 8H) 1.61-1.80 (m, 4 H) 1.87 (br d, J = 5.0 Hz, 2 H) 1.96 (br s, 1 H) 2.04 (br s, 1 H) 2.15 (br s, 1 H) 2.35-2.47 (m, 1 H) 2.53 (br s, 1 H) 2.80 (br d, J = 6.1 Hz, 2 H) 2.91- 2.99 (m, 1 H) 3.00-3.16 (m, 3 H) 3.25 (s, 3 H) 3.32 (s, 4 H) 3.35-3.48 (m, 6 H) 3.49- 3.57 (m, 4 H) 3.62-3.73 (m, 2 H) 3.94-4.03 (m, 1 H) 4.10 (br d, J = 9.0 Hz, 2 H) 4.70 (br d, J = 25.5 Hz, 1 H) 5.00-5.19 (m, 1 H) 6.72 (d, J = 8.3 Hz, 1 H) 7.08-7.18 (m, 2 H) 7.29 (s, 1 H) 7.63 (br s, 1 H); LCMS confirms the MW (Rₜ: 2.09, MW: 792.30, [M + H]⁺ 793, Method 3). |
| 18 | 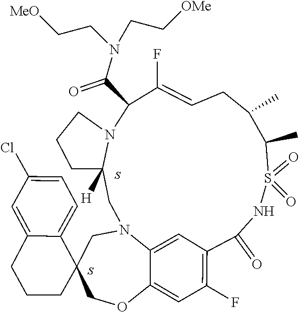 | ¹H NMR δ 1.08 (d, J = 6.7 Hz, 3 H) 1.39- 1.49 (m, 5 H) 1.59-1.74 (m, 5 H) 1.76-1.85 (m, 1 H) 2.04-2.09 (m, 2 H) 2.32 (br dd, J = 15.8, 7.8 Hz, 1 H) 2.70-2.82 (m, 2 H) 2.82- 2.93 (m, 1 H) 2.98-3.08 (m, 1 H) 3.08-3.15 (m, 1 H) 3.16-3.23 (m, 1 H) 3.30-3.38 (m, 4 H) 3.42 (s, 4 H) 3.53-3.64 (m, 4 H) 3.65- 3.77 (m, 2 H) 3.85 (br d, J = 14.3 Hz, 1 H) 3.97-4.07 (m, 2 H) 4.09-4.27 (m, 3 H) 4.68- 4.89 (m, 1 H) 5.07 (d, J = 24.1 Hz, 1 H) 6.74 (d, J = 11.2 Hz, 1 H) 7.04 (d, J = 7.0 Hz, 1 H) 7.10 (d, J = 2.3 Hz, 1 H) 7.21 (dd, J = 8.5, 2.3 Hz, 1 H) 7.66 (d, J = 8.6 Hz, 1 H) 8.03-8.34 (m, 1 H); LCMS confirms the MW (Rₜ: 2.20, Area %, MW: 792.00, [M + H]⁺ 793, Method 3). |

Compound 19

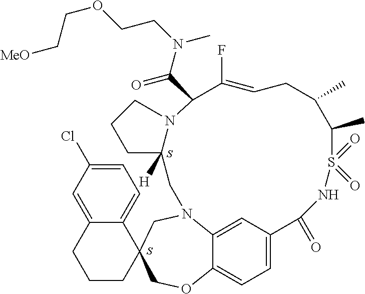

Propanephosphonic acid anhydride (59.5 μL, 0.10 mmol, 3.2 eq.) was added to a stirred solution of Intermediate 19 (34 mg, 0.032 mmol), [2-(2-methoxyethoxy)ethyl](methyl)amine (CAS [124192-94-5], 21 mg, 0.16 mmol, 5 eq.) and triethylamine (64 μL, 0.46 mmol, 14 eq.) in 2 mL of anhydrous DCM. The resulting mixture was stirred at room temperature for 1 h.

Upon completion of the reaction, the mixture was carefully quenched with 1M HCl sol. until pH reached ca 5-6. Water and DCM were added. The layers were separated and the aqueous layer was back-extracted with DCM (×3). The combined organic layers were dried (MgSO4) and evaporated under reduced pressure. A purification was performed via Prep HPLC (Stationary phase: RP XBridge Prep C18 OBD-10 μm, 30×150 mm, Mobile phase: 0.5% NH₄OAc solution in water+10% CH₃CN, CH₃CN) yielding Compound 19 (17.5 mg, yield 71%) as a pale orange solid.

¹H NMR δ ppm 1.05 (d, J=6.3 Hz, 4H) 1.34-1.47 (m, 5H) 1.60-1.75 (m, 3H) 1.76-1.86 (m, 1H) 1.90-2.10 (m, 5H) 2.29-2.40 (m, 1H) 2.71-2.83 (m, 2H) 2.83-2.98 (m, 2H) 2.99-3.04 (m, 1H) 3.10-3.20 (m, 1H) 3.27-3.41 (m, 6H) 3.49-3.79 (m, 7H) 4.06 (br s, 3H) 4.12-4.23 (m, 2H) 4.48-4.97 (m, 2H) 6.84-6.97 (m, 2H) 6.97-7.05 (m, 1H) 7.10 (d, J=2.1 Hz, 1H) 7.21 (dd, J=8.5, 2.2 Hz, 1H) 7.64-7.77 (m, 1H); LCMS confirms the MW (Rt: 2.13 min, Area %: 100.00, MW: 774.00, [M+H]⁺ 775, Method 4); SFC (Rt: 3.35, 0.00 isomer 1), (Rt: 3.94, 100.00% isomer 2) (Rₜ: 3.94 min, Area %: 100.00, Method 14).

The following compounds were made by analogy with Compound 18, following the pathway described in Scheme 2, and starting from the respective starting materials.

Table 3:

TABLE 3

| Compound | Formula | Analytical data |
|---|---|---|
| 20 | 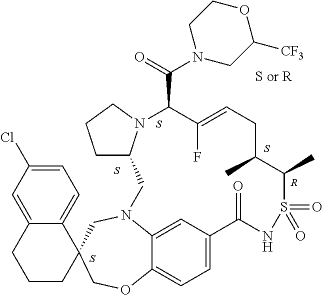 | ¹H NMR δ 0.99-1.11 (m, 3 H) 1.14- 1.23 (m, 2H) 1.37-1.47 (m, 4 H) 1.66-1.84 (m, 4 H) 1.88-2.09 (m, 5 H) 2.25-2.49 (m, 1 H) 2.72-3.04 (m, 5 H) 3.21-3.71 (m, 4 H) 3.80- 4.03 (m, 3 H) 4.03-4.20 (m, 3 H) 4.31-4.72 (m, 3 H) 4.89-5.15 (m, 1 H) 6.87-7.13 (m, 4 H) 7.17-7.23 (m, 1 H) 7.64-7.73 (m, 1 H); LCMS confirms the MW (Rₜ: 2.26, MW: 796.30, [M + H]⁺ 797, Method 3); SFC (Rₜ: 6.14, traces % isomer 1), (Rₜ: 6.87, 93.16% isomer 2) (Area %: 93.16, Method 15). |
| 21 | 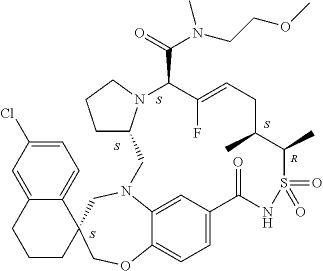 | ¹H NMR δ 1.06 (d, J = 6.2 Hz, 3 H) 1.36-1.42 (m, 1 H) 1.44 (d, J = 7.3 Hz, 3 H) 1.59-1.77 (m, 4 H) 1.78- 1.88 (m, 1 H) 1.89-2.12 (m, 5 H) 2.28-2.42 (m, 1 H) 2.70-2.84 (m, 2 H) 2.85-2.96 (m, 1 H) 2.97-3.45 (m, 10 H) 3.58 (s, 3 H) 3.92-4.21 (m, 5 H) 4.48-5.07 (m, 1 H) 4.75- 4.96 (m, 1 H) 6.86-6.92 (m, 1 H) 6.93-6.98 (m, 1 H) 7.00-7.08 (m, 1 H) 7.10 (d, J = 1.1 Hz, 1 H) 7.21 (dd, J = 8.6, 2.2 Hz, 1 H) 7.70 (d, J = 8.6 Hz, 1 H); LCMS confirms the MW (Rₜ: 2.14, MW: 730.00, [M + H]⁺ 731, Method 4). |
| 22 | 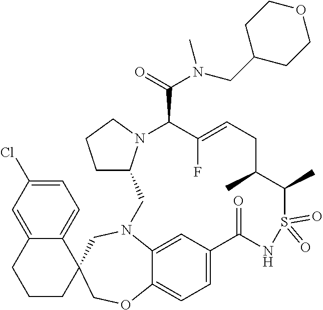 [SMILES: C[C@@H]1[C@@H](C)C/C=C(\F)[C@H](C(=O)N(C)CC2CCOCC2)N2CCC[C@H]2CN2C[C@@]3(CCCc4cc(Cl)ccc43)COc3ccc(cc32)C(=O)NS1(=O)=O] | ¹H NMRS δ 1.03-1.09 (m, 3 H) 1.17-1.40 (m, 5 H) 1.42-1.46 (m, 4 H) 1.54-1.75 (m, 6 H) 1.76-1.86 (m, 1 H) 1.90-2.05 (m, 6 H) 2.30- 2.39 (m, 1 H) 2.69-2.81 (m, 2 H) 2.87-2.99 (m, 3 H) 3.12-3.22 (m, 2 H) 3.29-3.46 (m, 5 H) 3.94-4.05 (m, 4 H) 4.11-4.22 (m, 2 H) 4.44-4.61 (m, 1 H) 4.78-4.96 (m, 1 H) 6.86- 6.91 (m, 1 H) 6.92-6.96 (m, 1 H) 6.96-7.03 (m, 1 H) 7.07-7.10 (m, 1 H) 7.18-7.23 (m, 1 H) 7.65-7.74 (m, 1 H); LCMS confirms the MW (Rₜ: 2.11, MW: 770.30, [M + H]⁺ 771, Method 3). |
| 23 | 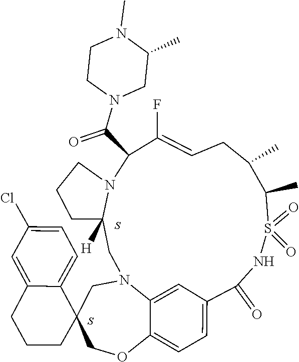 | ¹H NMR δ 1.05 (br t, J = 6.7 Hz, 3 H) 1.16 (dd, J = 45.0, 6.2 Hz, 3 H) 1.34-1.46 (m, 4 H) 1.58-1.75 (m, 3 H) 1.76-1.86 (m, 1 H) 1.91-2.11 (m, 6 H) 2.15-2.31 (m, 2 H) 2.31-2.35 (m, 3 H) 2.54-2.72 (m, 1 H) 2.73- 3.07 (m, 7 H) 3.08-3.16 (m, 1 H) 3.33 (br dd, J = 14.1, 2.5 Hz, 2 H) 3.87-4.00 (m, 3 H) 4.03-4.10 (m, 1 H) 4.15-4.24 (m, 1 H) 4.26-4.46 (m, 1 H) 4.46-4.66 (m, 1 H) 4.73-4.96 (m, 1 H) 6.86-6.96 (m, 2 H) 6.99- 7.06 (m, 1 H) 7.07-7.11 (m, 1 H) 7.16-7.22 (m, 1 H) 7.60-7.74 (m, 1 H); LCMS confirms the MW (Rₜ: 2.50, MW: 755, [M + H]⁺ 756, Method 5). |
| 24 | 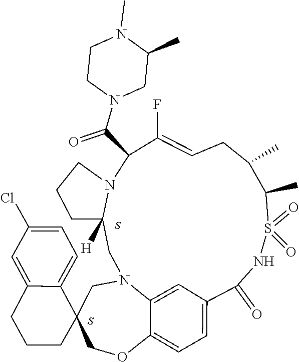 | ¹H NMR δ 1.02-1.08 (m, 3 H) 1.14 (br dd, J = 23.1, 6.1 Hz, 3 H) 1.36- 1.44 (m, 4 H) 1.62-1.72 (m, 3 H) 1.76-1.85 (m, 1 H) 1.92-2.06 (m, 5 H) 2.09-2.26 (m, 2 H) 2.31-2.37 (m, 3 H) 2.56-2.68 (m, 1 H) 2.72- 2.80 (m, 2 H) 2.82-2.87 (m, 1 H) 2.88-2.97 (m, 2 H) 3.00-3.16 (m, 2 H) 3.26-3.51 (m, 3 H) 3.86-4.04 (m, 3 H) 4.08-4.22 (m, 3 H) 4.31- 4.61 (m, 2 H) 4.76-4.93 (m, 1 H) 6.93 (s, 2 H) 6.97-7.04 (m, 1 H) 7.09 (d, J = 1.8 Hz, 1 H) 7.18-7.23 (m, 1 H) 7.69 (br d, J = 8.5 Hz, 1 H); LCMS confirms the MW (Rₜ: 2.51, MW: 755, [M + H]⁺ 756, Method 5). |
| 25 | 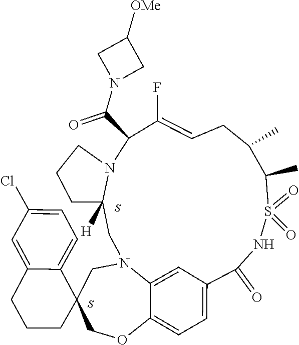 | ¹H NMR δ 1.03-1.06 (m, 3 H) 1.39- 1.45 (m, 4 H) 1.59-1.65 (m, 2 H) 1.65-1.72 (m, 2 H) 1.76-1.85 (m, 1 H) 1.90-1.98 (m, 2 H) 1.99-2.06 (m, 3 H) 2.27-2.36 (m, 1 H) 2.69- 2.82 (m, 2 H) 2.84-2.93 (m, 1 H) 2.94-3.03 (m, 1 H) 3.06-3.16 (m, 1 H) 3.23-3.36 (m, 5 H) 3.86-4.00 (m, 4 H) 4.07-4.15 (m, 1 H) 4.16- 4.22 (m, 2 H) 4.23-4.32 (m, 2 H) 4.43-4.61 (m, 1 H) 4.76-4.96 (m, 1 H) 6.89-6.96 (m, 2 H) 6.97-7.01 (m, 1 H) 7.07-7.11 (m, 1 H) 7.16- 7.22 (m, 1 H) 7.69 (d, J = 8.6 Hz, 1 H); LCMS confirms the MW (Rₜ: 2.05, MW: 728.00, [M + H]⁺ 729, Method 7). |
| 26 | 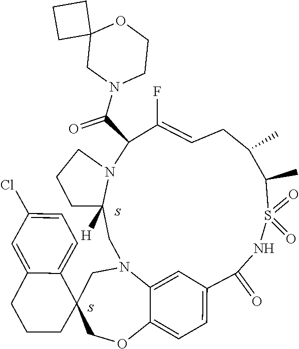 | LCMS confirms the MW (Rₜ: 2.27, MW: 768.30, [M + H]⁺ 769, Method 3). |
| 27 | 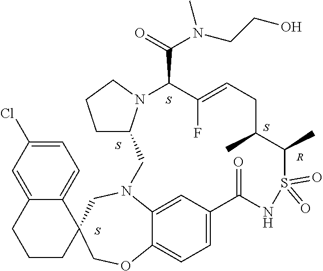 | ¹H NMR δ 1.06 (d, J = 6.2 Hz, 3 H) 1.44 (br d, J = 7.7 Hz, 4 H) 1.60- 1.77 (m, 2 H) 1.78-1.88 (m, 1 H) 2.04 (br s, 5 H) 2.23-2.41 (m, 1 H) 2.79 (br s, 3 H) 3.01 (s, 4 H) 3.07- 3.19 (m, 2 H) 3.21-3.29 (m, 1 H) 3.31-3.38 (m, 2 H) 3.42-3.58 (m, 1 H) 3.80 (br s, 2 H) 3.81-3.90 (m, 2 H) 3.91-4.07 (m, 3 H) 4.08-4.23 (m, 2 H) 4.53-5.09 (m, 2 H) 6.94 (s, 2 H) 7.01-7.13 (m, 2 H) 7.20 (br d, J = 8.4 Hz, 1 H) 7.69 (br d, J = 8.6 Hz, 1 H); LCMS confirms the MW (Rₜ: 2.44, MW: 716.00, [M + H]⁺ 717, Method 3) |
| 28 | 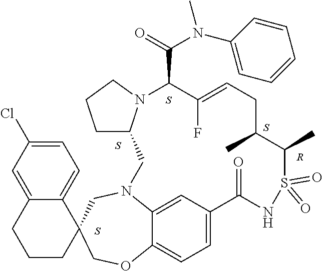 | ¹H NMR δ 0.98-1.06 (m, 3 H) 1.31- 1.41 (m, 2H) 1.41-1.48 (m, 3 H) 1.56-1.65 (m, 3 H) 1.71-1.78 (m, 1 H) 1.81-1.91 (m, 3 H) 1.93-1.99 (m, 1 H) 1.99-2.08 (m, 1 H) 2.24- 2.38 (m, 1 H) 2.41-2.57 (m, 1 H) 2.72-2.99 (m, 4 H) 3.09 (br d, J = 14.2 Hz, 1 H) 3.20-3.38 (m, 4 H) 3.78-4.05 (m, 3 H) 4.14 (br d, J = 12.1 Hz, 2 H) 4.40 (br d, J = 23.8 Hz, 1 H) 4.68-4.89 (m, 1 H) 6.38- 6.54 (m, 1 H) 6.74-6.95 (m, 2 H) 7.04-7.10 (m, 1 H) 7.14-7.21 (m, 1 H) 7.35 (br s, 3 H) 7.41-7.49 (m, 2 H) 7.57-7.65 (m, 1 H); LCMS confirms the MW (Rₜ: 2.30, MW: 748.00, [M + H]⁺ 749, Method 4). |
| 29 | 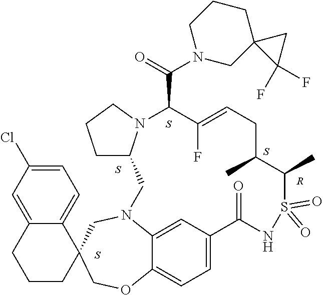 | ¹H NMR δ 1.02-1.16 (m, 4 H) 1.33- 1.46 (m, 5 H) 1.57-1.70 (m, 5 H) 1.73-1.84 (m, 4 H) 1.90-2.09 (m, 5 H) 2.14-2.43 (m, 1 H) 2.71-2.84 (m, 2 H) 2.85-3.18 (m, 3 H) 3.22- 3.36 (m, 2 H) 3.38-3.81 (m, 2 H) 3.82-4.22 (m, 6 H) 4.29-4.99 (m, 2 H) 6.84-7.04 (m, 3 H) 7.07-7.11 (m, 1 H) 7.17-7.23 (m, 1 H) 7.67 (s, 1 H); LCMS confirms the MW (Rₜ: 12.29, MW: 788.00, [M + H]⁺ 789, Method 4). |
| 30 | 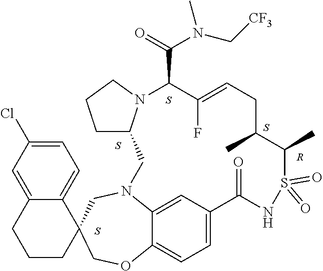 | ¹H NMR δ 0.98-1.09 (m, 3 H) 1.37- 1.47 (m, 4 H) 1.66-1.74 (m, 3 H) 1.76-1.85 (m, 1 H) 1.89-2.08 (m, 5 H) 2.28-2.45 (m, 1 H) 2.70-2.95 (m, 4 H) 3.04-3.20 (m, 2 H) 3.28- 3.45 (m, 4 H) 3.69-3.84 (m, 1 H) 3.85-4.03 (m, 3 H) 4.14-4.22 (m, 2 H) 4.31-4.45 (m, 1 H) 4.48-4.63 (m, 1 H) 4.84-5.00 (m, 1 H) 6.88- 7.01 (m, 3 H) 7.07-7.11 (m, 1 H) 7.17-7.24 (m, 1 H) 7.64-7.74 (m, 1 H) 8.18-8.51 (m, 1 H); LCMS confirms the MW (Rₜ: 2.25 min, MW: 754.00, [M + H]⁺ 755, Method 4); SFC (Rₜ: 4.64, 98.64%; Method 16) |
| 31 | 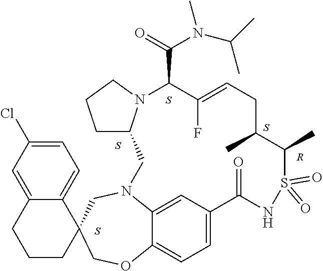 | ¹H NMR δ 0.99-1.07 (m, 3 H) 1.10- 1.15 (m, 6 H) 1.21-1.31 (m, 4 H) 1.33-1.45 (m, 4H) 1.65-1.72 (m, 2 H) 1.76-1.85 (m, 1 H) 1.88-1.95 (m, 1 H) 1.98-2.09 (m, 3 H) 2.17- 2.37 (m, 1 H) 2.69-2.84 (m, 3 H) 2.89-2.98 (m, 1 H) 3.01-3.05 (m, 1 H) 3.07-3.18 (m, 1 H) 3.29-3.55 (m, 2 H) 3.59-3.69 (m, 1 H) 3.86- 4.05 (m, 3 H) 4.12-4.22 (m, 1 H) 4.38-5.49 (m, 3 H) 6.75-6.97 (m, 2 H) 7.03-7.20 (m, 3 H) 7.60-7.76 (m, 1 H); LCMS confirms the MW (Rₜ: 2.20, MW: 714.00, [M + H]⁺ 715, Method 4). |
| 32 | 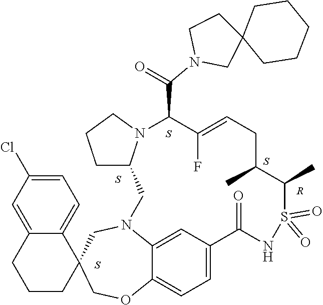 | ¹H NMR δ 1.06 (dd, J = 5.9, 3.7 Hz, 3 H) 1.21 (dd, J = 9.9, 6.4 Hz, 2 H) 1.39 (br dd, J = 7.2, 2.8 Hz, 10 H) 1.64-1.77 (m, 4 H) 1.84 (br t, J = 7.2 Hz, 1 H) 1.89-1.96 (m, 1 H) 1.96-2.13 (m, 4 H) 2.24-2.38 (m, 1 H) 2.70-2.85 (m, 2 H) 2.88-3.05 (m, 2 H) 3.06-3.21 (m, 1 H) 3.27-3.39 (m, 3 H) 3.43-3.51 (m, 1 H) 3.56 (s, 2 H) 3.61 (br d, J = 10.6 Hz, 1 H) 3.75 (br d, J = 10.3 Hz, 1 H) 3.82- 3.99 (m, 4 H) 3.99-4.06 (m, 1 H) 4.18 (dd, J = 12.3, 2.6 Hz, 1 H) 4.32-4.44 (m, 1 H) 4.91-5.17 (m, 1 H) 6.90 (d, J = 7.9 Hz, 1 H) 7.03 (td, J = 8.2, 1.4 Hz, 1 H) 7.09 (d, J = 2.0 Hz, 1 H) 7.10-7.13 (m, 1 H) 7.20 (dd, J = 8.6, 2.2 Hz, 1 H) 7.72 (d, J = 8.6 Hz, 1 H); LCMS confirms |
| | | the MW (Rₜ: 2.43, MW: 780.30, |
| | | [M + H]⁺ 781, Method 3); SFC (Rₜ: |
| | | 3.43, 0.00% isomer 1), (Rₜ: 4.04, |
| | | 100.00 % isomer 2) (Rt: 4.04, Area |
| | | %: 100.00, MW: 780.3, [M + H]⁺ |
| | | 781, Method 14) |
| 33 | 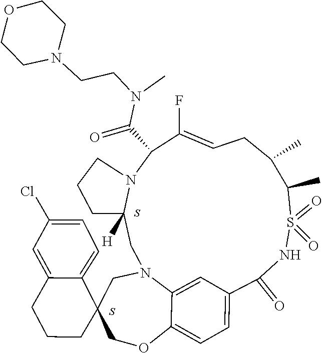 | ¹H NMR δ 1.05-1.11 (m, 3 H) 1.20- 1.25 (m, 5H) 1.31-1.36 (m, 3 H) 1.70-1.88 (m, 6 H) 2.23-2.30 (m, 1 H) 2.37-2.54 (m, 5 H) 2.73-2.80 (m, 2 H) 2.96-3.00 (m, 2 H) 3.14- 3.37 (m, 5 H) 3.38-3.46 (m, 1 H) 3.55-3.65 (m, 4 H) 3.67-3.72 (m, 2 H) 3.82-3.99 (m, 4 H) 4.13-4.18 (m, 1 H) 4.35-4.59 (m, 1 H) 4.79- 5.04 (m, 1 H) 6.83-6.90 (m, 1 H) 7.05-7.10 (m, 1 H) 7.15-7.23 (m, 2 H) 7.65-7.76 (m, 2 H); LCMS confirms the MW (Rₜ: 2.07, MW: 785.30, [M + H]⁺ 786, Method 3); SFC (Rt 4.60, 100.00% isomer 1), (Rₜ: 6.97, 0.00% isomer 2) (Rₜ: 4.60, Area %: 100.00, MW: 785.34, [M + H]⁺ 786, Method 11). |
| 34 | 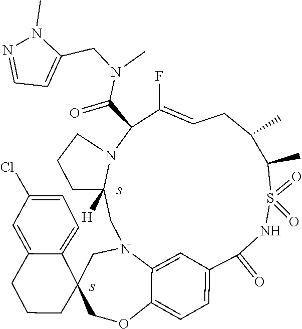 | ¹H NMR δ 1.06 (br d, J = 6.2 Hz, 3 H) 1.44 (d, J = 7.3 Hz, 3 H) 1.56- 1.85 (m, 6 H) 1.89-2.06 (m, 5 H) 2.36 (br dd, J = 13.8, 8.5 Hz, 1 H) 2.69-2.80 (m, 2 H) 2.81-2.96 (m, 2 H) 2.98-3.24 (m, 4 H) 3.33 (br d, J = 13.9 Hz, 2 H) 3.76-4.04 (m, 3 H) 3.86-3.93 (m, 2 H) 4.10-4.23 (m, 2 H) 4.34-5.13 (m, 3 H) 4.66-4.94 (m, 1 H) 6.07-6.29 (m, 1 H) 6.20-6.21 (m, 1 H) 6.87-7.03 (m, 3 H) 7.09 (d, J = 2.2 Hz, 1 H) 7.21 (dd, J = 8.4, 2.2 Hz, 1 H) 7.43 (d, J = 1.5 Hz, 1 H) 7.69 (d, J = 8.6 Hz, 1 H); LCMS confirms the MW (Rₜ: 2.07, MW: 766.00, [M + H]⁺ 767, Method 3). |
| 35 | 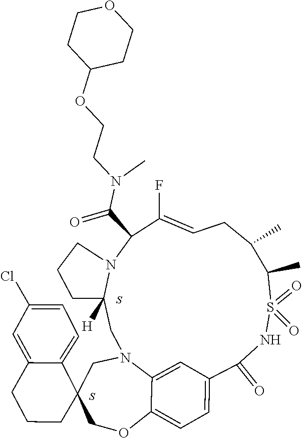 | ¹H NMR δ 1.02-1.10 (m, 3 H) 1.34- 1.41 (m, 1 H) 1.41-1.46 (m, 3 H) 1.49-1.73 (m, 6H) 1.76-2.11 (m, 10 H) 2.25-2.41 (m, 1 H) 2.72-2.99 (m, 4 H) 2.99-3.04 (m, 1 H) 3.10- 3.27 (m, 1 H) 3.29-3.33 (m, 1 H) 3.40-3.58 (m, 3 H) 3.61-3.77 (m, 3 H) 3.80-4.04 (m, 6 H) 4.08-4.22 (m, 2 H) 4.48-4.96 (m, 2 H) 6.86- 6.92 (m, 1 H) 6.92-6.97 (m, 1 H) 6.99-7.04 (m, 1 H) 7.05-7.12 (m, 1 H) 7.18-7.24 (m, 1 H) 7.65-7.74 (m, 1 H); LCMS confirms the MW (Rₜ: 1.15, MW: 800.00, [M + H]⁺ 801, Method 1). |
| 36 | 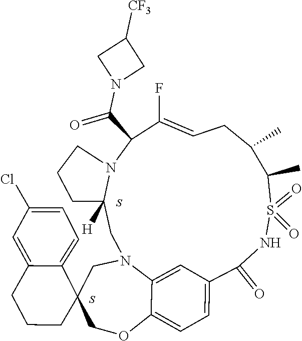 | ¹H NMR δ 0.98-1.08 (m, 4 H) 1.39- 1.46 (m, 3 H) 1.55-1.71 (m, 3 H) 1.75-2.08 (m, 7 H) 2.26-2.38 (m, 1 H) 2.71-3.01 (m, 4 H) 3.02-3.14 (m, 1 H) 3.26-3.39 (m, 3 H) 3.86- 4.00 (m, 3 H) 4.06-4.34 (m, 5 H) 4.43-4.63 (m, 1 H) 4.84-5.04 (m, 1 H) 6.86-6.97 (m, 2 H) 6.97-7.04 (m, 1 H) 7.06-7.13 (m, 1 H) 7.16- 7.23 (m, 1 H) 7.62-7.78 (m, 1 H); LCMS confirms the MW (Rₜ: 2.05, MW: 766.30, [M + H]⁺ 767, Method 4). |
| 37 | 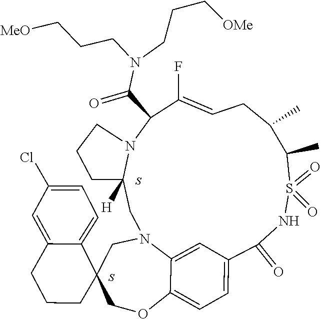 | ¹H NMR δ 1.01-1.08 (m, 3 H) 1.36- 1.46 (m, 4 H) 1.64-1.73 (m, 4 H) 1.80-1.89 (m, 3H) 1.91-2.10 (m, 6 H) 2.34-2.44 (m, 1 H) 2.69-2.81 (m, 2 H) 2.82-2.92 (m, 2 H) 3.13-3.26 (m, 2 H) 3.28-3.51 (m, 13 H) 3.58- 3.67 (m, 1 H) 3.92-4.12 (m, 4 H) 4.14-4.25 (m, 2 H) 4.49-4.66 (m, 1 H) 4.81-5.00 (m, 1 H) 6.83-6.89 (m, 1 H) 6.91-6.96 (m, 1 H) 7.01-7z.05 (m, 1 H) 7.07-7.10 (m, 1 H) 7.18- 7.23 (m, 1 H) 7.69 (d, J = 8.4 Hz, 1 H) 8.06 (br d, J = 1.5 Hz, 1 H); LCMS confirms the MW (Rₜ: 2.08, MW: 802.00, [M + H]⁺ 803, Method 4). |
| 38 | 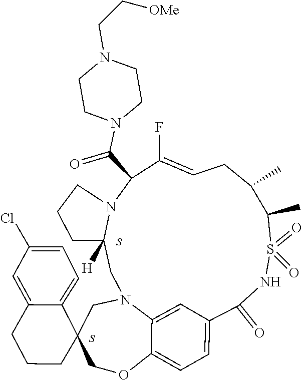 | ¹H NMR δ 1.03-1.09 (m, 3 H) 1.37- 1.45 (m, 4 H) 1.60-1.73 (m, 4 H) 1.77-1.87 (m, 1 H) 1.90-2.08 (m, 6 H) 2.27-2.37 (m, 1 H) 2.44-2.65 (m, 6 H) 2.70-2.83 (m, 2 H) 2.84- 2.93 (m, 1 H) 2.98-3.12 (m, 2 H) 3.27-3.35 (m, 2 H) 3.52-3.58 (m, 2 H) 3.67-3.76 (m, 3 H) 3.80-3.89 (m, 2 H) 3.90-4.02 (m, 3 H) 4.04- 4.14 (m, 1 H) 4.15-4.21 (m, 1 H) 4.46-4.61 (m, 1 H) 4.75-4.96 (m, 1 H) 6.88-6.96 (m, 2 H) 6.99-7.04 (m, 1 H) 7.06-7.12 (m, 1 H) 7.16- 7.22 (m, 1 H) 7.65-7.74 (m, 1 H); LCMS confirms the MW (Rₜ: 1.84, MW: 785.30, [M + H]⁺ 786, Method 8). |
| 39 | 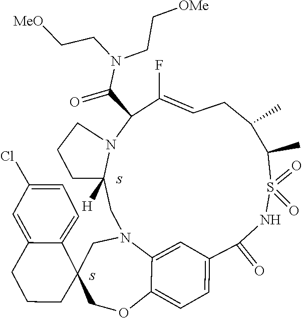 | ¹H NMR δ ppm 1.02-1.08 (m, 3 H) 1.41-1.47 (m, 3 H) 1.63-1.72 (m, 2 H) 1.78-1.89 (m, 3 H) 1.90-2.10 (m, 6 H) 2.28-2.39 (m, 1 H) 2.69- 2.83 (m, 2 H) 2.89-2.98 (m, 1 H) 3.00-3.10 (m, 1 H) 3.15-3.30 (m, 2 H) 3.32-3.35 (m, 3 H) 3.36-3.42 (m, 1 H) 3.42-3.45 (m, 3 H) 3.54- 3.68 (m, 5 H) 3.71-3.81 (m, 1 H) 3.93-4.03 (m, 2 H) 4.05-4.12 (m, 1 H) 4.13-4.20 (m, 2 H) 4.20-4.29 (m, 1 H) 4.77-4.96 (m, 1 H) 5.04- 5.18 (m, 1 H) 6.84-6.91 (m, 1 H) 6.92-6.97 (m, 1 H) 7.02-7.06 (m, 1 H) 7.07-7.12 (m, 1 H) 7.16-7.23 (m, 1 H) 7.65-7.74 (m, 1 H); LCMS confirms the MW (Rₜ: 2.19, MW: 774.30, [M + H]⁺ 775, Method 3). |
| 40 | 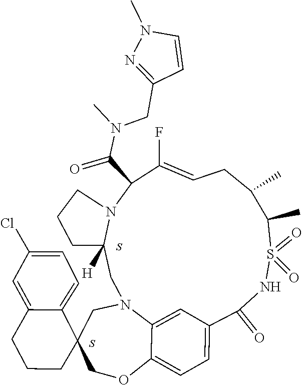 | ¹H NMR δ ppm 0.79-0.94 (m, 1 H) 1.04 (br t, J = 6.5 Hz, 3 H) 1.21-1.52 (m, 8 H) 1.64-2.13 (m, 11 H) 2.32- 2.52 (m, 1 H) 2.78 (br s, 5 H) 3.03 (s, 2 H) 3.22 (s, 2 H) 3.34 (br dd, J = 13.3, 10.2 Hz, 2 H) 3.87 (br d, J = 11.5 Hz, 3 H) 3.95 (br dd, J = 12.2, 7.0 Hz, 2 H) 4.10-4.36 (m, 3 H) 4.43-4.57 (m, 1 H) 4.64-5.04 (m, 2 H) 5.48 (br d, J = 16.2 Hz, 1 H) 6.20 (dd, J = 4.8, 1.9 Hz, 1 H) 6.81- 6.96 (m, 2 H) 7.08 (br s, 2 H) 7.20 (br d, J = 8.4 Hz, 1 H) 7.28-7.41 (m, 1 H) 7.69 (dd, J = 8.4, 4.1 Hz, 1 H); LCMS confirms the MW (Rₜ: 2.05, MW: 766.30, [M + H]⁺ 767, Method 3). |
| 41 | 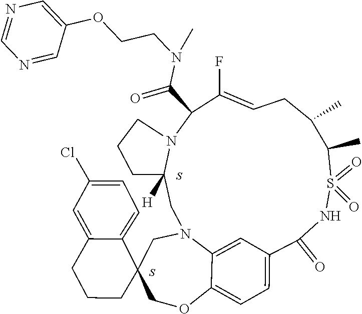 | ¹H NMR δ ppm 0.85-0.94 (m, 1 H) 1.03 (br d, J = 5.4 Hz, 3 H) 1.24- 1.33 (m, 3 H) 1.40 (br d, J = 7.3 Hz, 4 H) 1.63-1.79 (m, 4H) 1.90-2.12 (m, 5 H) 2.23-2.43 (m, 1 H) 2.69- 2.84 (m, 3 H) 2.91 (br d, J = 4.5 Hz, 2 H) 2.99-3.24 (m, 3 H) 3.33 (br d, J = 14.1 Hz, 2 H) 3.42 (s, 3 H) 3.70- 3.86 (m, 2 H) 3.87-4.04 (m, 4 H) 4.07-4.14 (m, 1 H) 4.16 (s, 1 H) 4.30 (s, 2 H) 4.37-4.45 (m, 1 H) 4.55 (d, J = 24.0 Hz, 1 H) 4.81-4.92 (m, 1 H) 4.93-5.00 (m, 1 H) 6.93 (s, 2 H) 7.00 (s, 1 H) 7.07-7.11 (m, 1 H) 7.20 (dd, J = 8.4, 2.2 Hz, 1 H) 7.65-7.75 (m, 1 H) 8.41-8.59 (m, 2 H) 8.79-8.90 (m, 1 H); LCMS confirms the MW (Rₜ: 2.01, MW: 794.30, [M + H]⁺ 795, Method 3). |
| 42 | 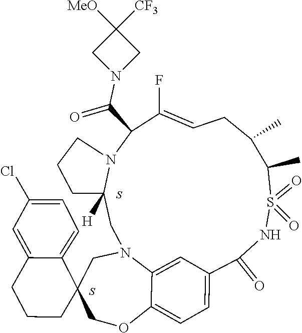 | ¹H NMR δ ppm 1.04-1.08 (m, 3 H) 1.42-1.47 (m, 3 H) 1.62-1.73 (m, 4 H) 1.77-1.88 (m, 1 H) 1.92-2.11 (m, 5 H) 2.28-2.43 (m, 1 H) 2.70-3.02 (m, 4 H) 3.04-3.13 (m, 1 H) 3.25- 3.38 (m, 2 H) 3.50-3.56 (m, 3 H) 3.86-4.02 (m, 3 H) 4.13-4.27 (m, 4 H) 4.28-4.34 (m, 1 H) 4.38-4.51 (m, 1 H) 4.52-4.60 (m, 1 H) 4.80-5.06 (m, 1 H) 6.86-6.91 (m, 1 H) 6.92- 6.98 (m, 2 H) 7.08-7.13 (m, 1 H) 7.18-7.24 (m, 1 H) 7.64-7.73 (m, 1 H) 8.00-8.16 (m, 1 H); LCMS confirms the MW (Rₜ: 1.24, MW: 796.00, [M + H]⁺ 797, Method 1). |
| 43 | 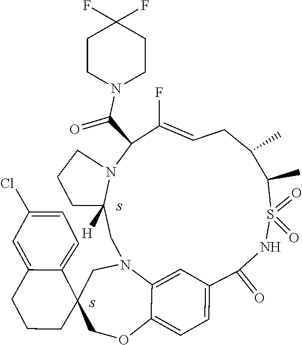 | ¹H NMR δ ppm 1.02-1.06 (m, 3 H) 1.21-1.27 (m, 2H) 1.41-1.45 (m, 3 H) 1.63-1.81 (m, 4 H) 1.95-2.01 (m, 4 H) 2.26-2.36 (m, 1 H) 2.73- 2.81 (m, 2 H) 2.85-2.94 (m, 1 H) 2.95-3.03 (m, 1 H) 3.06-3.14 (m, 1 H) 3.26-3.37 (m, 2 H) 3.42-3.53 (m, 1 H) 3.54-3.63 (m, 1 H) 3.67- 3.76 (m, 2 H) 3.76-3.89 (m, 4 H) 3.90-3.93 (m, 1 H) 3.94-3.97 (m, 1 H) 3.97-4.01 (m, 1 H) 4.08-4.15 (m, 1 H) 4.16-4.22 (m, 1 H) 4.50- 4.69 (m, 1 H) 4.78-4.96 (m, 1 H) 6.93-6.97 (m, 2 H) 6.97-7.01 (m, 1 H) 7.07-7.12 (m, 1 H) 7.16-7.23 (m, 1 H) 7.62-7.75 (m, 1 H) 8.17- 9.01 (m, 1 H); LCMS confirms the MW (Rₜ: 1.21, MW: 762.00, [M + H]⁺ 763, Method 1). |
| 44 | 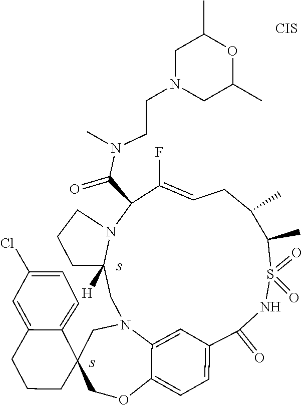 | ¹H NMR δ ppm 0.96-1.01 (m, 1 H) 1.02-1.12 (m, 4 H) 1.14-1.18 (m, 4 H) 1.40-1.46 (m, 3 H) 1.64-1.75 (m, 3 H) 1.76-1.87 (m, 3 H) 1.90-2.08 (m, 6 H) 2.29-2.41 (m, 2 H) 2.47- 2.57 (m, 2 H) 2.57-2.66 (m, 1 H) 2.73-2.82 (m, 4 H) 2.83-2.98 (m, 3 H) 3.08-3.16 (m, 1H) 3.31-3.45 (m, 3 H) 3.59-3.71 (m, 3 H) 3.89-4.02 (m, 3 H) 4.05-4.12 (m, 1 H) 4.15- 4.21 (m, 1 H) 4.44-4.67 (m, 1 H) 4.81-5.01 (m, 1 H) 6.89-6.96 (m, 2 H) 7.00-7.05 (m, 1 H) 7.07-7.12 (m, 1 H) 7.16-7.23 (m, 1 H) 7.61-7.77 (m, 1 H); LCMS confirms the MW (Rₜ: 2.19, MW: 813.30, [M + H]⁺ 814, Method 3); SFC (Rₜ: 6.22, Area %: 100.00, Method 17). |
| 45 | 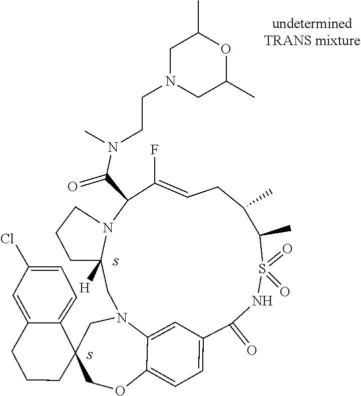 | ¹H NMR δ ppm 1.01-1.08 (m, 2 H) 1.09-1.18 (m, 2 H) 1.26 (s, 10 H) 1.37-1.46 (m, 3 H) 1.87-2.09 (m, 5 H) 2.15-2.25 (m, 2 H) 2.26-2.39 (m, 1 H) 2.43-2.65 (m, 3 H) 2.70-2.82 (m, 2 H) 2.85-2.99 (m, 2 H) 3.11- 3.19 (m, 1 H) 3.26-3.38 (m, 3 H) 3.43-3.81 (m, 5 H) 3.87-4.03 (m, 4 H) 4.08-4.22 (m, 2 H) 4.45-4.56 (m, 1 H) 4.77-5.04 (m, 2 H) 6.88-6.97 (m, 2 H) 6.98-7.06 (m, 1 H) 7.07- 7.12 (m, 1 H) 7.17-7.24 (m, 1 H) 7.65-7.75 (m, 1 H); LCMS confirms the MW (Rₜ: 2.22, MW: 813.30, [M + H]⁺ 814, Method 3); SFC (Rₜ: 6.23 min 8.95% CIS), (Rₜ: 6.37 min 91.05% TRANS) (Method 17). |
| 46 | 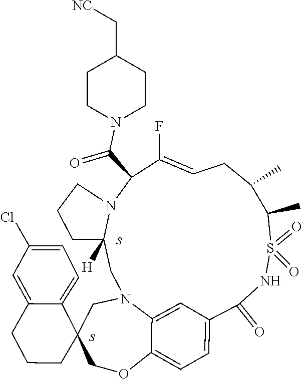 | ¹H NMR δ ppm 1.00-1.12 (m, 3 H) 1.44 (br t, J = 7.2 Hz, 5 H) 1.63-1.76 (m, 3 H) 1.78-2.13 (m, 10 H) 2.37 (dt, J = 6.4, 3.1 Hz, 3 H) 2.58-2.69 (m, 1 H) 2.72-3.04 (m, 4 H) 3.05- 3.38 (m, 4 H) 3.96 (s, 3 H) 4.13 (br dd, J = 15.1, 7.4 Hz, 1 H) 4.17-4.23 (m, 1 H) 4.35-4.96 (m, 4 H) 6.95 (br s, 2 H) 7.01 (br d, J = 7.9 Hz, 1 H) 7.10 (br s, 1 H) 7.21 (dd, J = 8.5, 1.9 Hz, 1 H) 7.69 (d, J = 8.4 Hz, 1 H) 7.89-9.09 (m, 1 H); LCMS confirms the MW (Rₜ: 2.12, MW: 765.30, [M + H]⁺ 766, Method 3). |
| 47 | 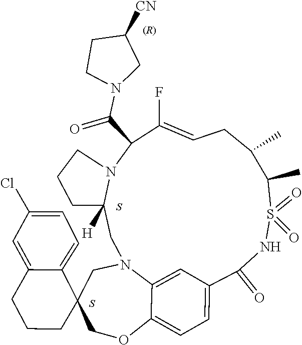 | ¹H NMR δ ppm 1.06 (d, J = 6.4 Hz, 3 H) 1.44 (s, 4 H) 1.60-1.76 (m, 4 H) 1.80-1.91 (m, 1 H) 1.95-2.10 (m, 4 H) 2.22-2.46 (m, 3 H) 2.72-2.85 (m, 2 H) 2.87-2.99 (m, 3 H) 3.14- 3.25 (m, 1 H) 3.26-3.41 (m, 2 H) 3.63 (s, 6 H) 4.10-4.45 (m, 4 H) 4.81-5.01 (m, 1 H) 6.89-6.94 (m, 1 H) 6.94-7.03 (m, 2 H) 7.11 (d, J = 2.4 Hz, 1 H) 7.22 (dd, J = 8.5, 2.3 Hz, 1 H) 7.70 (d, J = 8.4 Hz, 1 H) 8.02 (s, 1 H); LCMS confirms the MW (Rt: 2.46, MW: 737.30, [M + H]⁺: 738, Method 3). |
| 48 | 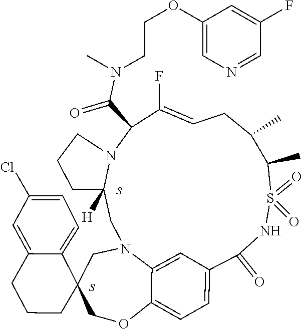 | ¹H NMR δ ppm 0.81-0.91 (m, 1 H) 1.03 (br d, J = 6.0 Hz, 3 H) 1.13 (d, J = 6.2 Hz, 2 H) 1.35-1.44 (m, 4 H) 1.62-1.70 (m, 3 H) 1.72-1.82 (m, 1 H) 1.95-2.04 (m, 3 H) 2.32 (br dd, J = 13.7, 8.5 Hz, 1 H) 2.72-2.80 (m, 2 H) 2.87-2.96 (m, 2 H) 3.04-3.13 (m, 2 H) 3.28-3.37 (m, 2 H) 3.42 (s, 2 H) 3.68-3.74 (m, 1 H) 3.88-3.97 (m, 2 H) 3.99 (s, 2 H) 4.12-4.19 (m, 2 H) 4.22 (br t, J = 5.1 Hz, 1 H) 4.55 (d, J = 24.4 Hz, 1 H) 4.77-4.98 (m, 1 H) 6.93 (s, 2 H) 6.96-7.01 (m, 2 H) 7.08 (d, J = 2.3 Hz, 1 H) 7.19 (dd, J = 8.4, 2.2 Hz, 1 H) 7.65-7.71 (m, 1 H) 8.06-8.12 (m, 1H) 8.16 (d, J = 1.7 Hz, 1 H); LCMS confirms the MW (Rₜ: 2.13, MW: 811.30, [M + H]⁺ 812, Method 3). |
| 49 | 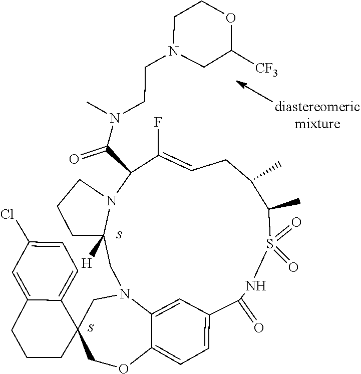 | ¹H NMR δ ppm 1.04-1.10 (m, 3 H) 1.42-1.47 (m, 3 H) 1.67-1.70 (m, 1 H) 1.88-2.09 (m, 8 H) 2.19-2.41 (m, 4 H) 2.57-2.67 (m, 1 H) 2.70- 2.83 (m, 3 H) 2.86-3.00 (m, 3 H) 3.11-3.20 (m, 1 H) 3.26-3.30 (m, 2 H) 3.31-3.39 (m, 2 H) 3.41-3.54 (m, 1 H) 3.61-3.82 (m, 5 H) 3.92- 4.05 (m, 4 H) 4.12-4.22 (m, 2 H) 4.47-4.72 (m, 1 H) 4.79-4.98 (m, 1 H) 6.87-6.92 (m, 1 H) 6.94-6.97 (m, 1 H) 6.98-7.03 (m, 1 H) 7.06- 7.12 (m, 1 H) 7.15-7.23 (m, 1 H) 7.62-7.73 (m, 1 H) 7.82-8.38 (m, 1 H); LCMS confirms the MW (RT: 2.10, MW: 853.30, [M + H]⁺ 854, Method 6); SFC (Rt: 5.63 min 36.48% isomer 1), (Rt: 5.79 min 63.52% isomer 2) (RT: 5.63, Area %: 36.48, Method 11). |
| 50 | 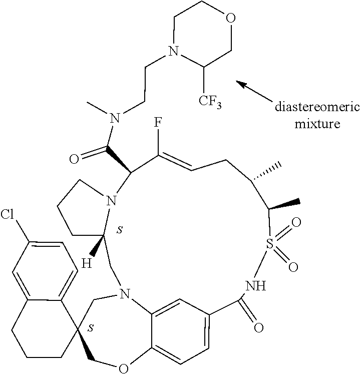 | ¹H NMR δ ppm 1.04-1.09 (m, 3 H) 1.42-1.46 (m, 3 H) 1.95-2.06 (m, 4 H) 2.07-2.14 (m, 1 H) 2.19-2.41 (m, 2 H) 2.56-2.69 (m, 1 H) 2.72-2.81 (m, 2 H) 2.88-3.00 (m, 5 H) 3.05- 3.21 (m, 4 H) 3.24-3.30 (m, 3 H) 3.32-3.42 (m, 2 H) 3.49-3.57 (m, 1 H) 3.58-3.67 (m, 2 H) 3.68-3.84 (m, 4 H) 3.89-4.03 (m, 5 H) 4.12-4.23 (m, 2 H) 4.46-4.62 (m, 1 H) 4.77- 5.01 (m, 1 H) 6.87-6.91 (m, 1 H) 6.92-6.96 (m, 1 H) 6.97-7.03 (m, 1 H) 7.07-7.11 (m, 1 H) 7.17-7.23 (m, 1 H) 7.64-7.73 (m, 1 H); LCMS confirms the MW (Rₜ: 2.54, MW: 853.00, [M + H]⁺ 854, Method 9). |
| 51 | 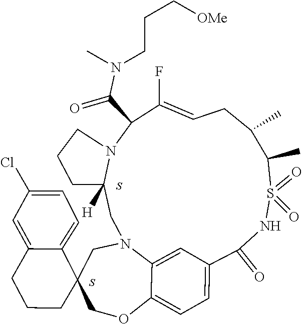 | ¹H NMR δ ppm 1.02-1.07 (m, 3 H) 1.38-1.47 (m, 4H) 1.62-1.74 (m, 3 H) 1.78-1.88 (m, 2 H) 1.90-1.96 (m, 2 H) 1.97-2.08 (m, 3 H) 2.29-2.41 (m, 1 H) 2.70-2.80 (m, 2 H) 2.83- 2.98 (m, 4 H) 3.10-3.26 (m, 3 H) 3.30-3.48 (m, 8 H) 3.87-4.07 (m, 4 H) 4.08-4.16 (m, 1 H) 4.16-4.23 (m, 1 H) 4.46-4.64 (m, 1 H) 4.81-4.98 (m, 1 H) 6.86-6.95 (m, 2 H) 7.03- 7.11 (m, 2 H) 7.16-7.21 (m, 1 H) 7.65-7.74 (m, 1 H); LCMS confirms the MW (RT: 2.14, MW: 744.30, [M + H]⁺ 745, Method 3); SFC (Rₜ: 3.49min 0.00% isomer 1), (Rt 4.20 min 100.00% isomer 2) (RT: 4.20, Area %: 100.00, Method 14). |
| 52 | 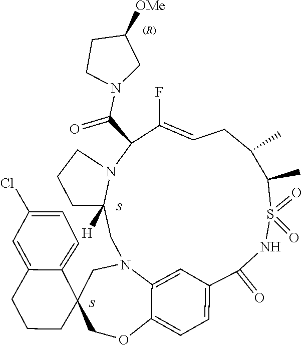 | ¹H NMR δ ppm 0.99-1.07 (m, 3 H) 1.39-1.47 (m, 4H) 1.62-1.73 (m, 3 H) 1.76-1.85 (m, 1 H) 1.89-1.97 (m, 2 H) 1.97-2.02 (m, 2 H) 2.04-2.12 (m, 2 H) 2.13-2.22 (m, 1 H) 2.25- 2.40 (m, 1 H) 2.71-2.82 (m, 2 H) 2.85-3.01 (m, 2 H) 3.10-3.21 (m, 1 H) 3.29-3.42 (m, 5 H) 3.49-3.57 (m, 1 H) 3.66-3.75 (m, 2 H) 3.87- 4.11 (m, 5H) 4.12-4.22 (m, 2 H) 4.38-4.57 (m, 1 H) 4.78-5.00 (m, 1 H) 6.91-6.96 (m, 2 H) 7.02-7.09 (m, 2 H) 7.19 (dd, J = 8.5, 2.2 Hz, 1 H) 7.65-7.73 (m, 1 H); LCMS confirms the MW (Rₜ: 2.17, MW: 742.00, [M + H]⁺ 743, Method 3). |
| 53 | 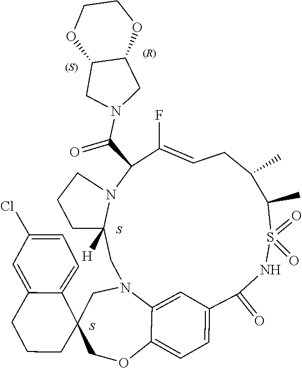 | ¹H NMR δ ppm 1.03-1.08 (m, 3 H) 1.31-1.34 (m, 1 H) 1.41-1.46 (m, 3 H) 1.53-1.60 (m, 1H) 1.61-1.75 (m, 4H) 1.76-1.86 (m, 1 H) 1.86- 2.11 (m, 6 H) 2.33-2.42 (m, 1 H) 2.70-2.87 (m, 3 H) 2.89-2.94 (m, 1 H) 3.15-3.25 (m, 1 H) 3.28-3.38 (m, 2 H) 3.54-3.62 (m, 1 H) 3.62- 3.73 (m, 3 H) 3.73-3.81 (m, 1 H) 3.83-3.94 (m, 3 H) 3.94-4.09 (m, 3 H) 4.12-4.32 (m, 1 H) 4.33-4.43 (m, 1 H) 4.78-5.02 (m, 1 H) 6.86- 6.92 (m, 1 H) 6.92-6.97 (m, 1 H) 6.98-7.03 (m, 1 H) 7.06-7.12 (m, 1 H) 7.16-7.23 (m, 1 H) 7.65-7.73 (m, 1 H) 7.94-8.24 (m, 1 H); LCMS confirms the MW (Rₜ: 1.09, MW: 770.00, [M + H]⁺ 771, Method 1). |
| 54 | 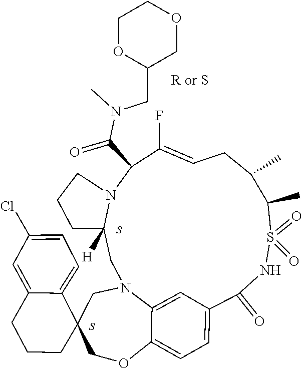 | ¹H NMR δ ppm 1.00-1.07 (m, 3 H) 1.39-1.48 (m, 4H) 1.65-1.76 (m, 3 H) 1.80-2.08 (m, 6 H) 2.31-2.44 (m, 1 H) 2.71-2.88 (m, 3 H) 2.89-2.95 (m, 1 H) 2.95-3.03 (m, 2 H) 3.10- 3.20 (m, 1 H) 3.22-3.39 (m, 5 H) 3.48-3.63 (m, 2 H) 3.66-3.80 (m, 3 H) 3.81-3.93 (m, 1 H) 3.94-3.98 (m, 1 H) 4.02 (d, J = 12.1 Hz, 1 H) 4.09- 4.27 (m, 3 H) 4.48 (s, 1 H) 4.81-4.98 (m, 1 H) 5.04-5.16 (m, 1 H) 6.84- 6.91 (m, 1 H) 6.92-6.97 (m, 1 H) 7.01 (s, 1 H) 7.09 (s, 1 H) 7.18-7.24 (m, 1 H) 7.66-7.72 (m, 1 H); LCMS confirms the MW: (Rₜ: 2.14, MW: 772.00, [M + H]⁺ 773, Method 3). |
| 55 | 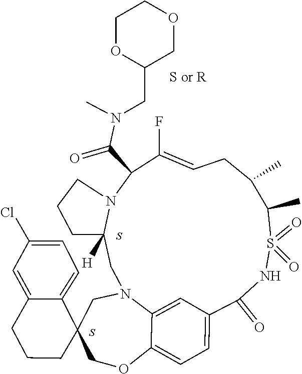 | ¹H NMR δ ppm 1.01-1.09 (m, 3 H) 1.38-1.45 (m, 4H) 1.57-1.62 (m, 1 H) 1.65-1.75 (m, 3 H) 1.76-1.84 (m, 2 H) 1.88-2.00 (m, 3 H) 2.01- 2.10 (m, 2 H) 2.19-2.40 (m, 1 H), 2.78 (br d, J = 3.1 Hz, 2 H) 2.99 (s, 3 H) 3.06-3.22 (m, 2 H) 3.31-3.36 (m, 3 H) 3.40-3.64 (m, 2 H) 3.65- 3.81 (m, 5 H) 3.81-3.87 (m, 1 H) 3.91-4.01 (m, 2 H) 4.08-4.20 (m, 2 H) 4.47-5.14 (m, 2 H) 6.86-6.97 (m, 2 H) 6.99-7.13 (m, 2 H) 7.17- 7.22 (m, 1 H) 7.68 (s, 1 H); LCMS confirms the MW (Rₜ: 2.11, MW: 772.00, [M + H]⁺ 773, Method 3). |
| 56 | 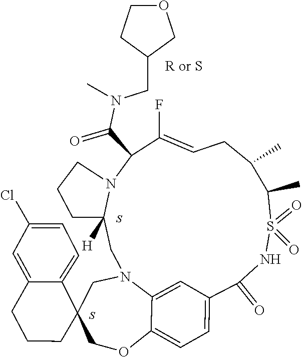 | ¹H NMR δ ppm 0.95-1.00 (m, 1 H) 1.02-1.07 (m, 3H) 1.36-1.40 (m, 3 H) 1.64-1.75 (m, 4 H) 1.77-1.87 (m, 1 H) 1.90-2.08 (m, 5 H) 2.09-2.16 (m, 1 H) 2.25-2.33 (m, 1 H) 2.59- 2.68 (m, 1 H) 2.73-2.83 (m, 2 H) 3.10-3.23 (m, 2 H) 3.29-3.38 (m, 2 H) 3.47-3.57 (m, 2 H) 3.59-3.72 (m, 2 H) 3.73-3.81 (m, 2 H) 3.82-4.05 (m, 7 H) 4.12-4.19 (m, 1 H) 4.47- 4.58 (m, 2 H) 4.96-5.12 (m, 1 H) 6.86-6.91 (m, 1 H) 7.04-7.14 (m, 3 H) 7.17-7.23 (m, 1 H) 7.68-7.74 (m, 1 H); LCMS confirms the MW (Rₜ: 2.12, MW: 756.30, [M + H]⁺ 757, Method 3). |
| 57 | 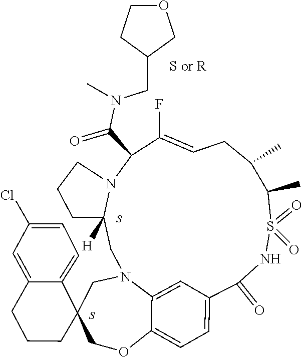 | ¹H NMR δ ppm 1.02-1.07 (m, 3 H) 1.36-1.41 (m, 3 H) 1.59-1.75 (m, 5 H) 1.78-1.86 (m, 1 H) 1.90-2.16 (m, 7 H) 2.25-2.33 (m, 1 H) 2.59-2.68 (m, 1 H) 2.73-2.83 (m, 2 H) 2.88- 2.99 (m, 3 H) 3.10-3.20 (m, 1 H) 3.32-3.38 (m, 1 H) 3.43-3.52 (m, 3 H) 3.58-3.70 (m, 1 H) 3.73-3.81 (m, 2 H) 3.82-3.90 (m, 2 H) 3.91-4.01 (m, 4 H) 4.14-4.19 (m, 1 H) 4.48- 4.57 (m, 1 H) 4.92-5.10 (m, 1 H) 6.87-6.95 (m, 1 H) 7.00-7.07 (m, 1 H) 7.07-7.12 (m, 2 H) 7.17-7.22 (m, 1 H) 7.68-7.73 (m, 1 H); LCMS confirms the MW (RT: 2.10, MW: 756.30, [M + H]⁺ 757, Method 3). |
| 58 | 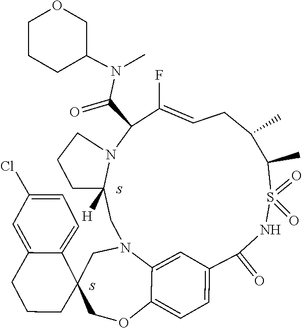 | ¹H NMR δ ppm 0.98-1.07 (m, 3 H) 1.37-1.47 (m, 4 H) 1.52-1.74 (m, 5 H) 1.77-2.09 (m, 8 H) 2.31-2.43 (m, 1 H) 2.71-2.81 (m, 2 H) 2.82-2.96 (m, 3 H) 3.12-3.20 (m, 3 H) 3.30- 3.39 (m, 2 H) 3.44-3.62 (m, 2 H) 3.91-4.21 (m, 7 H) 4.45-4.64 (m, 1 H) 4.67-4.80 (m, 1 H) 4.82-4.98 (m, 1 H) 6.91-7.02 (m, 3 H) 7.10 (d, J = 2.2 Hz, 1 H) 7.18-7.24 (m, 1 H) 7.66-7.75 (m, 1 H); LCMS confirms the MW (Rₜ: 1.97, MW: 756.00, [M + H]⁺ 757, Method 4). |
| 59 | 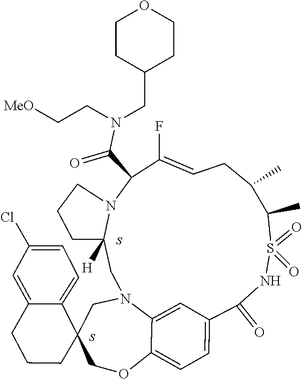 | ¹H NMR δ ppm 1.06 (d, J = 6.2 Hz, 3 H) 1.32-1.48 (m, 6 H) 1.54-1.75 (m, 6 H) 1.78-1.91 (m, 1 H) 1.91- 2.13 (m, 6 H) 2.29-2.43 (m, 1 H) 2.67-2.83 (m, 2 H) 2.83-3.03 (m, 3 H) 3.12-3.27 (m, 2 H) 3.31-3.40 (m, 4 H) 3.45 (s, 3 H) 3.55-3.62 (m, 2 H) 3.82-4.34 (m, 8 H) 4.51-5.19 (m, 2 H) 6.83-7.06 (m, 3 H) 7.10 (d, J = 2.0 Hz, 1 H) 7.21 (dd, J = 8.5, 2.3 Hz, 1 H) 7.70 (d, J = 8.6 Hz, 1 H) 7.84-8.58 (m, 1 H); LCMS confirms the MW (Rₜ: 2.24, MW: 814.00, [M + H]⁺ 815, Method 3). |
| 60 | 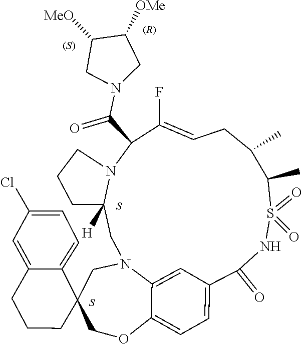 | ¹H NMR δ ppm 1.02 (br d, J = 6.4 Hz, 3 H) 1.42 (br d, J = 7.3 Hz, 4 H) 1.56-1.85 (m, 5 H) 2.01 (s, 5 H) 2.24-2.41 (m, 1 H) 2.67-2.99 (m, 4 H) 3.00-3.15 (m, 1 H) 3.33 (br d, J = 14.3 Hz, 2 H) 3.42-3.56 (m, 6 H) 3.58-3.89 (m, 3 H) 3.88-4.05 (m, 5 H) 4.10-4.23 (m, 2 H) 4.36-4.50 (m, 1 H) 4.76-5.00 (m, 1 H) 6.89-7.06 (m, 3 H) 7.09 (d, J = 2.2 Hz, 1 H) 7.19 (dd, J = 8.4, 1.8 Hz, 1 H) 7.65- 7.74 (m, 1 H); LCMS confirms the MW, (Rₜ: 2.05, MW: 772.30, [M + H]⁺ 773, Method 3). |
| 61 | 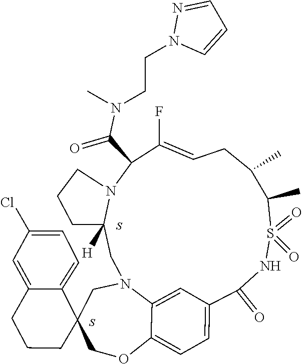 | ¹H NMR δ ppm 0.88 (s, 1 H) 1.05 (br dd, J = 8.7, 6.5 Hz, 3 H) 1.20- 1.36 (m, 6 H) 1.38-1.49 (m, 4 H) 1.60-1.89 (m, 5 H) 1.90-2.20 (m, 5 H) 2.32 (br dd, J = 13.3, 8.9 Hz, 1 H) 2.71-3.01 (m, 8 H) 3.07 (br d, J = 5.9 Hz, 1 H) 3.23-3.42 (m, 2 H) 3.65 (br d, J = 14.7 Hz, 1 H) 3.74-4.03 (m, 5 H) 4.04-4.25 (m, 2 H) 4.29- 4.56 (m, 3 H) 4.62-5.04 (m, 1 H) 4.95-4.97 (m, 1 H) 6.25 (s, 1 H) 6.91 (br s, 2 H) 6.98 (s, 1 H) 7.09 (s, 1 H) 7.20 (dd, J = 8.4, 2.4 Hz, 1 H) 7.34-7.43 (m, 1 H) 7.55 (d, J = 1.5 Hz, 1 H) 7.64-7.76 (m, 1 H) 7.93 (s, 1 H); LCMS confirms the MW (Rₜ: 2.10, MW: 766.30, [M + H]⁺ 767, BPM2: 765, Method 3). |
| 62 | 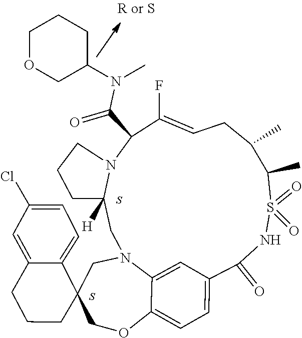 | ¹H NMR δ ppm 1.03-1.08 (m, 3 H) 1.18-1.23 (m, 3H) 1.34-1.40 (m, 3 H) 1.68-1.74 (m, 3 H) 1.78-1.86 (m, 3 H) 1.89-2.12 (m, 5 H) 2.20-2.35 (m, 2 H) 2.67-2.80 (m, 2 H) 2.89- 2.97 (m, 1 H) 3.03-3.11 (m, 1 H) 3.26-3.39 (m, 3 H) 3.40-3.52 (m, 2 H) 3.64-3.78 (m, 2 H) 3.85-4.07 (m, 6 H) 4.12-4.23 (m, 2 H) 4.46-4.60 (m, 1 H) 4.94-5.13 (m, 1 H) 6.83- 6.92 (m, 1 H) 7.01-7.12 (m, 2 H) 7.13-7.25 (m, 2 H) 7.67-7.76 (m, 1 H); LCMS confirms the MW (Rₜ: 2.17, MW: 756.30, [M + H]⁺ 757, Method 3); SFC (Rₜ: 4.37 min 100.00% isomer 1), (Rₜ: 4.69 min 0.00% isomer 2) (Rₜ: 4.37, Area %: 100.00, MW: 756.3, BPM1: 757, BPM2: 755, Method 14). |
| 63 | 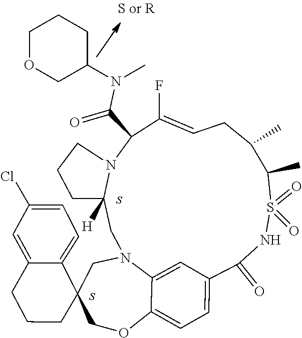 | ¹H NMR δ ppm 1.02-1.08 (m, 3 H) 1.33-1.38 (m, 3 H) 1.62-1.74 (m, 4 H) 1.74-1.84 (m, 3 H) 1.85-1.96 (m, 3 H) 1.97-2.06 (m, 3 H) 2.11-2.34 (m, 2 H) 2.70-2.82 (m, 2 H) 2.89- 3.02 (m, 2 H) 3.27-3.39 (m, 3 H) 3.40-3.46 (m, 1 H) 3.47-3.62 (m, 1 H) 3.79-4.06 (m, 8 H) 4.13-4.19 (m, 1 H) 4.19-4.27 (m, 1 H) 4.46-4.57 (m, 2 H) 4.95-5.13 (m, 1 H) 6.85- 6.91 (m, 1 H) 7.05-7.12 (m, 2 H) 7.12-7.22 (m, 2 H) 7.69-7.75 (m, 1 H); LCMS confirms the MW (Rₜ: 2.17, MW: 756.30, [M + H]⁺ 757, Method 3). |
| 64 | 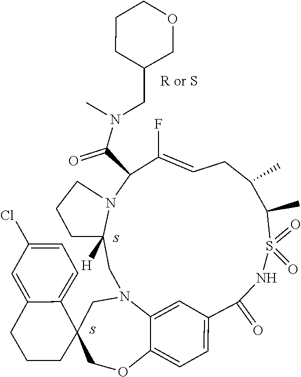 | ¹H NMR δ ppm 1.02-1.07 (m, 3 H) 1.17-1.22 (m, 1 H) 1.40-1.44 (m, 3 H) 1.54-1.73 (m, 6 H) 1.74-1.86 (m, 2 H) 1.88-2.11 (m, 7 H) 2.26-2.36 (m, 1 H) 2.70-2.82 (m, 2 H) 2.88- 3.02 (m, 3 H) 3.10-3.19 (m, 2 H) 3.41 (br s, 6 H) 3.79-3.86 (m, 2 H) 3.90-4.07 (m, 4 H) 4.14-4.21 (m, 1 H) 4.45-4.61 (m, 1 H) 4.83-5.02 (m, 1 H) 6.88-6.98 (m, 2 H) 7.02-7.06 (m, 1 H) 7.07-7.10 (m, 1 H) 7.17- 7.22 (m, 1 H) 7.66-7.72 (m, 1 H); LCMS confirms the MW (Rₜ: 2.19, MW: 770.30, [M + H]⁺ 771, Method 3); SFC (Rₜ: 6.78 min 100.00% isomer 1), (Rt: 8.04 min 0.00% isomer 2) manual integration (Rₜ: 6.78, Area %: 100.00, MW: 770.33, BPM1: 771, BPM2: 769, Method 11). |
| 65 | 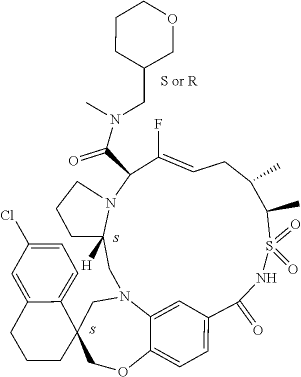 | ¹H NMR δ ppm 1.01-1.06 (m, 3 H) 1.18-1.24 (m, 3 H) 1.35-1.41 (m, 4 H) 1.52-1.73 (m, 6 H) 1.89-2.02 (m, 4 H) 2.07-2.15 (m, 1 H) 2.23-2.36 (m, 1 H) 2.69-2.85 (m, 2 H) 2.87- 2.94 (m, 1 H) 3.05-3.13 (m, 1 H) 3.13-3.22 (m, 2 H) 3.31-3.43 (m, 2 H) 3.44-3.60 (m, 3 H) 3.68-4.04 (m, 9 H) 4.13-4.21 (m, 1 H) 4.46-4.66 (m, 1 H) 4.91-5.14 (m, 1 H) 6.85- 6.90 (m, 1 H) 7.01-7.07 (m, 1 H) 7.06-7.09 (m, 1 H) 7.09-7.14 (m, 1 H) 7.16-7.22 (m, 1 H) 7.67-7.73 (m, 1 H); LCMS confirms the MW (Rₜ: 2.19, MW: 770.30, [M + H]⁺ 771, Method 3); SFC (Rt: 6.79 min traces% isomer 1), (Rₜ: 8.04 min 100.00% isomer 2) (Rₜ: 8.02, Area %: 100.00, MW: 770.33, BPM1: 771, BPM2: 769, Method 11). |
| 66 | 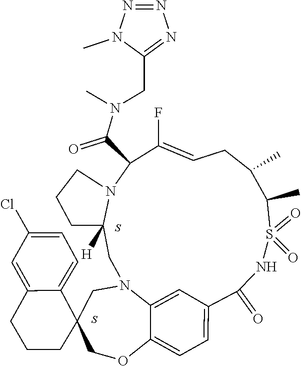 | ¹H NMR δ ppm 1.02 (br d, J = 6.16 Hz, 3 H) 1.33-1.42 (m, 4 H) 1.90- 2.00 (m, 5 H) 2.04 (br d, J = 3.96 Hz, 1 H) 2.17 (s, 4 H) 2.24-2.34 (m, 1 H) 2.68-2.80 (m, 3 H) 2.82-2.96 (m, 2 H) 2.96-3.04 (m, 1 H) 3.31 (br d, J = 14.30 Hz, 1 H) 3.34-3.41 (m, 1 H) 3.44 (s, 3 H) 3.89-3.99 (m, 3 H) 4.13 (br s, 1 H) 4.16 (s, 2 H) 4.55 (br d, J = 23.33 Hz, 1 H) 4.62 (br d, J = 14.74 Hz, 1 H) 4.98-5.07 (m, 1 H) 5.34-5.47 (m, 1 H) 6.85 (br d, J = 5.72 Hz, 1 H) 6.92 (br d, J = 6.82 Hz, 1 H) 7.01 (br s, 1 H) 7.08 (d, J = 1.98 Hz, 1 H) 7.17 (br d, J = 8.58 Hz, 1 H) 7.68 (d, J = 8.58 Hz, 1 H); LCMS confirms the MW (Rₜ: 1.94, MW: 768.30, [M + H]⁺ 769, Method 6). |
| 67 | 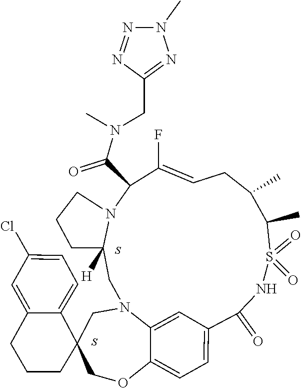 | LCMS confirms the MW, (Rₜ: 1.98, MW: 768.30, [M + H]⁺ 769, Method 6). |
| 68 | 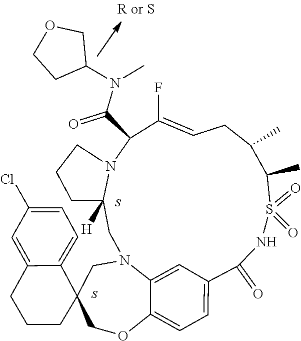 | ¹H NMR δ ppm 1.02-1.08 (m, 3 H) 1.29-1.36 (m, 2 H) 1.41-1.45 (m, 3 H) 1.57-1.75 (m, 4 H) 1.75-1.86 (m, 1 H) 1.88-2.08 (m, 7 H) 2.19-2.44 (m, 3 H) 2.72-2.82 (m, 2 H) 3.05- 3.19 (m, 2 H) 3.29-3.41 (m, 2 H) 3.64-3.86 (m, 4 H) 3.89-4.02 (m, 3 H) 4.04-4.15 (m, 2 H) 4.16-4.22 (m, 1 H) 4.41-4.72 (m, 1 H) (4.81-5.09 (m, 1 H) 6.89-6.97 (m, 2 H) 7.00- 7.07 (m, 1 H) 7.08-7.11 (m, 1 H) 7.17-7.22 (m, 1 H) 7.67-7.72 (m, 1 H); LCMS confirms the MW (Rₜ: 2.10, MW: 742.30, [M + H]⁺ 743, Method 3); SFC (Rₜ: 6.18 min 100.00% isomer 1), (Rₜ: 6.78 min 0.00% isomer 2) (Rₜ: 6.18, Area %: 100.00, MW: 742.30, BPM1: 743, BPM2: 741, Method 11). |
| 69 | 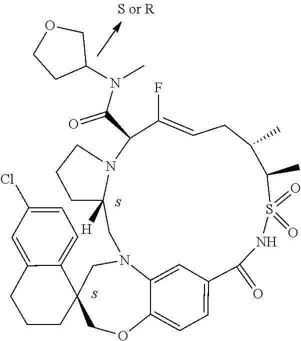 | ¹H NMR δ ppm 1.02-1.09 (m, 3 H) 1.31-1.34 (m, 1 H) 1.40-1.46 (m, 3 H) 1.63-1.75 (m, 4 H) 1.76-1.88 (m, 2 H) 1.91-2.08 (m, 6 H) 2.18-2.40 (m, 3 H) 2.70-2.81 (m, 2 H) 3.02- 3.16 (m, 1 H) 3.31-3.43 (m, 2 H) 3.63-3.72 (m, 1 H) 3.72-3.86 (m, 3 H) 3.87-4.02 (m, 4 H) 4.03-4.13 (m, 2 H) 4.15-4.22 (m, 1 H) 4.45-4.70 (m, 1 H) 4.80-5.11 (m, 2 H) 6.90- 7.00 (m, 2 H) 7.01-7.06 (m, 1 H) 7.08-7.11 (m, 1 H) 7.17-7.23 (m, 1 H) 7.67-7.74 (m, 1 H); LCMS confirms the MW (RT: 2.10, MW: 742.30, [M + H]⁺ 743, Method 3). |
| 70 | 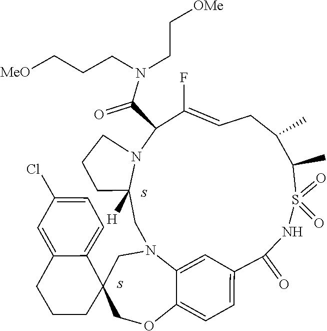 | ¹H NMR δ ppm 1.05 (br d, J = 5.9 Hz, 3 H) 1.36 (br s, 1 H) 1.42 (br d, J = 7.0 Hz, 3 H) 1.60-1.74 (m, 3 H) 1.84 (td, J = 12.8, 6.7 Hz, 2 H) 1.92- 2.08 (m, 5 H) 2.26-2.44 (m, 1 H) 2.69-2.81 (m, 2 H) 2.83-3.00 (m, 2 H) 3.08-3.18 (m, 1 H) 3.20-3.31 (m, 2 H) 3.32-3.47 (m, 10 H) 3.50-3.79 (m, 4 H) 3.93-4.21 (m, 6 H) 4.57 (br d, J = 23.3 Hz, 0.4 H) 4.76-5.00 (m, 1 H) 5.04 (br d, J = 25.5 Hz, 0.6 H) 6.86-6.94 (m, 2 H) 7.04-7.11 (m, 2 H) 7.20 (dd, J = 8.5, 2.1 Hz, 1 H) 7.70 (d, J = 8.6 Hz, 1 H); LCMS confirms the MW (RT: 1.20, MW: 789.00, [M + H]⁺ 789, Method 1). |
| 71 | 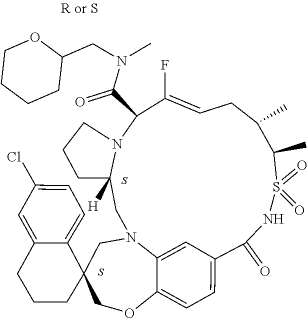 | ¹H NMR δ ppm 0.98-1.07 (m, 3 H) 1.43 (s, 4 H) 1.46-1.61 (m, 5 H) 1.62-1.77 (m, 4 H) 1.78-2.08 (m, 8 H) 2.33-2.46 (m, 1 H) 2.73-2.93 (m, 4 H) 3.03-3.13 (m, 1 H) 3.14-3.29 (m, 2 H) 3.35-3.39 (m, 1 H) 3.41- 3.74 (m, 2 H) 3.91-4.06 (m, 3 H) 4.13-4.24 (m, 3 H) 4.32-4.58 (m, 2 H) 4.80-5.01 (m, 1 H) 5.06-5.58 (m, 1 H) 6.85-6.96 (m, 2 H) 6.98- 7.04 (m, 1 H) 7.06-7.12 (m, 1 H) 7.17-7.24 (m, 1 H) 7.67-7.73 (m, 1 H) 7.87-8.63 (m, 1 H); LCMS confirms the MW (Rₜ: 1.26, MW: 770.00, [M + H]⁺ 771, Method 1). |
| 72 | 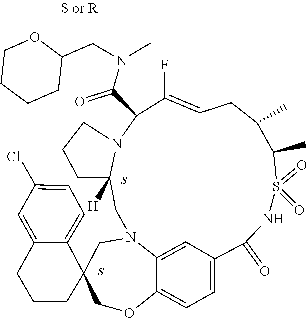 | ¹H NMR δ ppm 1.02-1.08 (m, 3 H) 1.36-1.41 (m, 2 H) 1.41-1.45 (m, 3 H) 1.47-1.53 (m, 2 H) 1.54-1.66 (m, 4 H) 1.67-1.77 (m, 3 H) 1.78-1.87 (m, 2 H) 1.88-2.11 (m, 6 H) 2.18- 2.27 (m, 1 H) 2.28-2.43 (m, 1 H) 2.68-2.81 (m, 2 H) 2.85-2.98 (m, 2 H) 3.11-3.20 (m, 1 H) 3.21-3.31 (m, 1 H) 3.36-3.47 (m, 1 H) 3.53-3.66 (m, 1 H) 3.76-3.85 (m, 1 H) 3.91- 4.03 (m, 4 H) 4.11-4.22 (m, 2 H) 4.45-4.57 (m, 1 H) 4.63-4.98 (m, 1 H) 4.98-5.10 (m, 1 H) 6.85-6.90 (m, 1 H) 6.91-6.96 (m, 1 H) 6.98-7.12 (m, 2 H) 7.18-7.23 (m, 1 H) 7.67- 7.72 (m, 1 H); LCMS confirms the MW (Rₜ: 1.24, MW: 770.00, [M + H]⁺ 771, Method 1). |
| 73 | 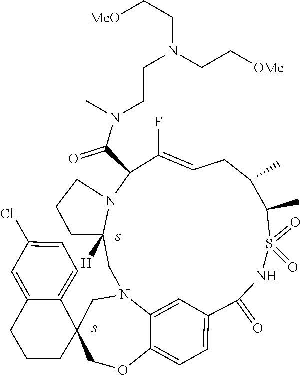 | ¹H NMR δ ppm 1.05 (dd, J = 6.5, 3.9 Hz, 3 H) 1.35-1.46 (m, 4 H) 1.58-1.65 (m, 1 H) 1.67-1.75 (m, 2 H) 1.75-1.85 (m, 1 H) 1.86-2.13 (m, 6 H) 2.24 (br s, 2 H) 2.72-2.83 (m, 6 H) 2.86-2.99 (m, 3 H) 3.02-3.19 (m, 3 H) 3.35 (s, 10 H) 3.49 (t, J = 5.8 Hz, 2 H) 3.53-3.65 (m, 2 H) 3.89-4.02 (m, 3 H) 4.10-4.25 (m, 2 H) 4.47-4.74 (m, 1 H) 4.77-4.96 (m, 1 H) 5.31-5.66 (m, 1 H) 6.89-7.24 (m, 5 H) 7.65-7.78 (m, 1 H); LCMS confirms the MW (Rₜ: 2.08, MW: 831.00, [M + H]⁺ 832, Method 6). |
| 74 | 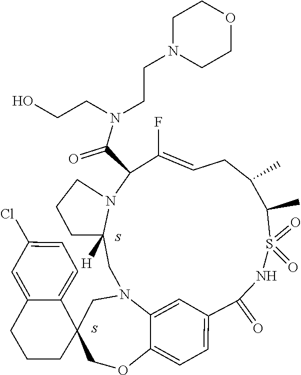 | ¹H NMR δ ppm 1.06 (br d, J = 5.7 Hz, 3 H) 1.34-1.40 (m, 1 H) 1.44 (br d, J = 7.0 Hz, 3 H) 1.60-1.83 (m, 4 H) 2.05 (s, 5 H) 2.23-2.39 (m, 1 H) 2.58 (br s, 4 H) 2.68-2.84 (m, 4 H) 2.94 (br dd, J = 15.0, 10.3 Hz, 1 H) 3.02-3.17 (m, 2 H) 3.19-3.39 (m, 3 H) 3.46-3.53 (m, 1 H) 3.54-3.63 (m, 1 H) 3.74 (br s, 6 H) 3.81-3.88 (m, 1 H) 3.98 (br s, 4 H) 4.07-4.22 (m, 2 H) 4.54-5.01 (m, 2 H) 6.87-7.05 (m, 3 H) 7.09 (d, J = 2.2 Hz, 1 H) 7.20 (dd, J = 8.6, 2.2 Hz, 1 H) 7.69 (d, J = 8.6 Hz, 1 H); LCMS confirms the MW (Rₜ: 1.08, MW: 815.00, [M + H]⁺ 814, Method 2). |
| 75 | 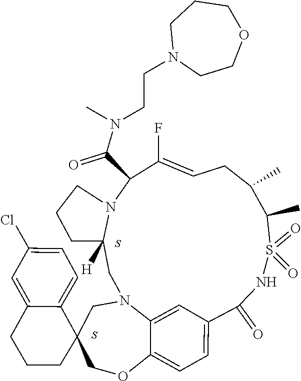 | ¹H NMR δ ppm 0.78-0.93 (m, 1 H) 1.02-1.09 (m, 3 H) 1.38-1.46 (m, 4 H) 1.60-1.74 (m, 3 H) 1.76-1.84 (m, 1 H) 1.87-1.95 (m, 3 H) 1.95-2.06 (m, 4 H) 2.28-2.40 (m, 1 H) 2.69- 2.84 (m, 7 H) 2.86-2.99 (m, 4 H) 3.10-3.18 (m, 1 H) 3.30-3.37 (m, 2 H) 3.37-3.48 (m, 1 H) 3.56-3.67 (m, 1 H) 3.70-3.82 (m, 4 H) 3.87-4.04 (m, 4 H) 4.10-4.22 (m, 2 H) 4.46- 4.73 (m, 1 H) 4.77-4.98 (m, 1 H) 6.86-6.96 (m, 2 H) 7.00-7.11 (m, 2 H) 7.18-7.23 (m, 1 H) 7.65-7.73 (m, 1 H); LCMS confirms the MW (Rₜ: 1.13, MW: 799.00, [M + H]⁺ 800, Method 1). |
| 76 | 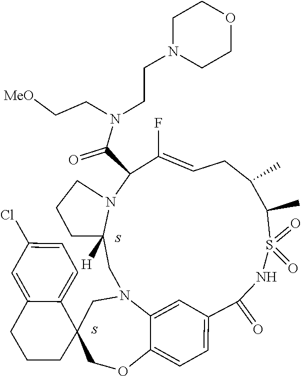 | ¹H NMR δ ppm 1.06 (d, J = 6.2 Hz, 3 H) 1.40-1.43 (m, 1 H) 1.44 (d, J = 7.3 Hz, 3 H) 1.62-1.75 (m, 3 H) 1.78-1.88 (m, 1 H) 1.93-2.10 (m, 6 H) 2.30-2.39 (m, 1 H) 2.46-2.67 (m, 6 H) 2.76-2.83 (m, 2 H) 2.85-2.94 (m, 1 H) 2.95-3.06 (m, 1 H) 3.08- 3.18 (m, 1 H) 3.20-3.29 (m, 1 H) 3.30-3.36 (m, 2 H) 3.36-3.47 (m, 3 H) 3.54-3.67 (m, 4 H) 3.68-3.74 (m, 3 H) 3.74-3.82 (m, 1 H) 3.95- 4.09 (m, 3 H) 4.12-4.27 (m, 3 H) 4.55-5.09 (m, 2 H) 6.86-6.97 (m, 2 H) 7.02-7.07 (m, 1 H) 7.10 (d, J = 2.2 Hz, 1 H) 7.21 (dd, J = 8.6, 2.2 Hz, 1 H) 7.70 (d, J = 8.4 Hz, 1 H); LCMS confirms the MW, (Rₜ: 2.12, MW: 829.40, [M + H]⁺ 830, Method 3). |
| 77 | 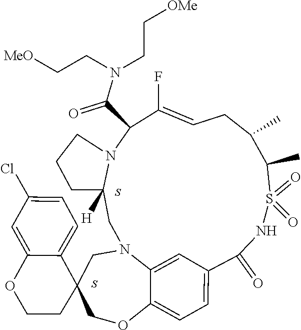 | ¹H NMR δ ppm 1.05 (d, J = 6.4 Hz, 3 H) 1.43 (d, J = 7.0 Hz, 3 H) 1.61- 1.73 (m, 3 H) 1.75-1.84 (m, 1 H) 1.88-2.07 (m, 4 H) 2.35 (br dd, J = 14.5, 8.6 Hz, 1 H) 2.79 (dd, J = 15.1, 10.2 Hz, 1 H) 2.92-3.01 (m, 1 H) 3.10-3.19 (m, 1 H) 3.23-3.31 (m, 1 H) 3.33 (s, 3 H) 3.32-3.41 (m, 2 H) 3.43 (s, 3 H) 3.53-3.61 (m, 5 H) 3.71-3.81 (m, 1 H) 4.00-4.21 (m, 6 H) 4.22-4.38 (m, 2 H) 4.85 (br dd, J = 36.6, 5.2 Hz, 1 H) 5.06 (br d, J = 24.9 Hz, 1 H) 6.84 (d, J = 2.2 Hz, 1 H) 6.90-6.98 (m, 3 H) 7.09 (s, 1 H) 7.60 (d, J = 8.4 Hz, 1 H); LCMS confirms the MW (Rₜ: 1.10, MW: 777.00, [M + H]⁺ 777, Method 1). |
| 78 | 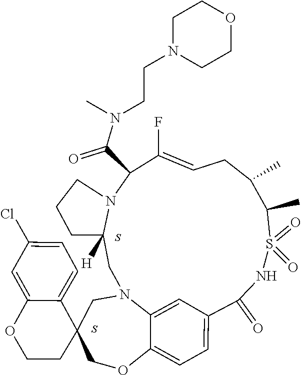 | ¹H NMR δ ppm 1.05 (br d, J = 6.4 Hz, 3 H) 1.43 (br d, J = 7.0 Hz, 3 H) 1.64-1.76 (m, 3 H) 1.76-1.86 (m, 1 H) 1.88-1.96 (m, 1 H) 1.96-2.08 (m, 3 H) 2.36 (br dd, J = 14.9, 8.7 Hz, 1 H) 2.45-2.61 (m, 6 H) 2.63-2.72 (m, 1 H) 2.82 (br dd, J = 15.1, 10.2 Hz, 1 H) 3.00 (s, 1 H) 3.09-3.20 (m, 1 H) 3.26 (s, 2 H) 3.33-3.46 (m, 3 H) 3.60-3.64 (m, 1 H) 3.69 (t, J = 4.4 Hz, 3 H) 3.91-4.25 (m, 7 H) 4.28- 4.37 (m, 1 H) 4.51 (d, J = 23.5 Hz, 0.75 H) 4.61 (br d, J = 22.7 Hz, 0.25 H) 4.81-4.96 (m, 1 H) 6.85 (d, J = 2.2 Hz, 1 H) 6.92-6.98 (m, 3 H) 7.05-7.11 (m, 1 H) 7.59 (d, J = 8.4 Hz, 1 H); LCMS confirms the MW (Rₜ: 1.03, MW: 788.00, [M + H]⁺ 788, Method 1). |
| 79 | 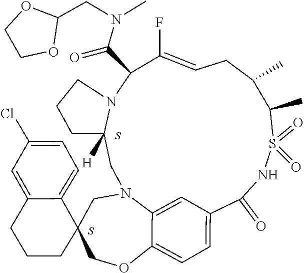 | ¹H NMR δ ppm 1.05 (d, J = 6.5 Hz, 3 H) 1.22 (dd, J = 6.3, 5.4 Hz, 1 H) 1.35-1.48 (m, 4 H) 1.62-1.78 (m, 3 H) 1.77-1.90 (m, 1 H) 1.91-2.11 (m, 5 H) 2.33 (br d, J = 9.5 Hz, 1 H) 2.78 (br d, J = 4.1 Hz, 2 H) 2.86- 2.98 (m, 2 H) 3.06 (s, 3 H) 3.11- 3.23 (m, 1 H) 3.26-3.50 (m, 3 H) 3.56 (br d, J = 2.0 Hz, 1 H) 3.85- 4.21 (m, 10 H) 4.34 (br dd, J = 15.7, 3.3 Hz, 1H) 5.04 (s, 2 H) 5.10-5.20 (m, 1 H) 6.92 (s, 2 H) 7.05 (s, 1 H) 7.09 (d, J = 2.2 Hz, 1 H) 7.20 (dd, J = 8.5, 2.1 Hz, 1 H) 7.70 (d, J = 8.6 Hz, 1 H); LCMS confirms the MW (RT: 2.12, MW: 758.30, [M + H]⁺ 759, Method 3). |
| 80 | 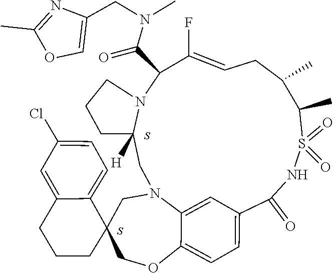 | LCMS confirms the MW (Rₜ: 2.01, MW: 767.3, [M + H]⁺ 768, Method 4). |
| 81 | 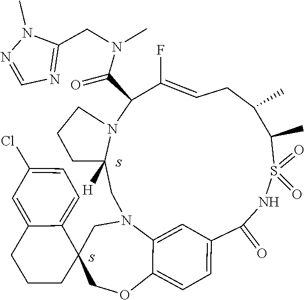 | LCMS confirms the MW (Rₜ: 1.90, MW: 767.30, [M + H]⁺ 768, Method 4). |
| 82 | 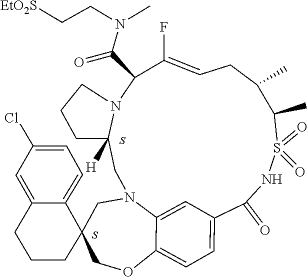 | LCMS confirms the MW (Rₜ: 1.97, MW: 792.30, [M + H]⁺ 793, Method 4). |
| 83 | 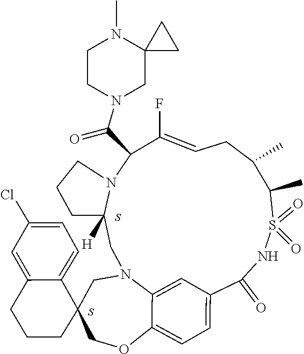 | ¹H NMR δ ppm 0.49-0.82 (m, 4 H) 1.05 (br d, J = 5.9 Hz, 3 H) 1.36-1.42 (m, 1 H) 1.44 (br d, J = 6.7 Hz, 3 H) 1.59-1.75 (m, 3 H) 1.75-1.87 (m, 1 H) 1.90-2.10 (m, 5 H) 2.21-2.37 (m, 1 H) 2.45 (d, J = 5.3 Hz, 3 H) 2.65- 2.81 (m, 2 H) 2.82-2.97 (m, 3 H) 2.98-3.13 (m, 2 H) 3.15-3.29 (m, 1 H) 3.33 (br dd, J = 14.3, 9.2 Hz, 1 H) 3.38-3.67 (m, 3 H) 3.68-3.89 (m, 2 H) 3.89-4.02 (m, 2 H) 4.03-4.16 (m, 1 H) 4.20 (br d, J = 12.3 Hz, 1 H) 4.48 (br dd, J = 55.8, 24.9 Hz, 1 H) 4.66-4.94 (m, 1 H) 6.87-7.05 (m, 3 H) 7.10 (s, 1 H) 7.21 (br d, J = 8.5 Hz, 1 H) 7.68 (t, J = 8.3 Hz, 1 H); LCMS confirms the MW (Rₜ: 1.14, MW: 767.00, [M + H]⁺ 768, Method 1); OR −46.61° (589 nm, c 0.19 w/v %, DMF, 20° C.). |
| 84 | 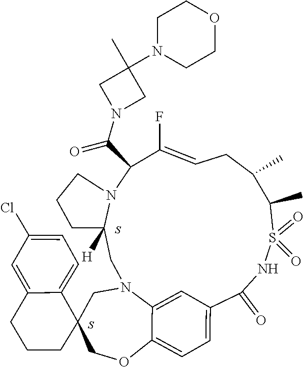 | ¹H NMR δ ppm 1.07-1.12 (m, 1 H) 1.23-1.31 (m, 2 H) 1.33-1.38 (m, 3 H) 1.42-1.46 (m, 3 H) 1.54-1.74 (m, 5 H) 1.76-1.87 (m, 1 H) 1.91-2.08 (m, 5 H) 2.27-2.37 (m, 1 H) 2.45- 2.50 (m, 3 H) 2.70-2.81 (m, 2 H) 2.84-2.94 (m, 1 H) 2.97-3.06 (m, 1 H) 3.07-3.16 (m, 1 H) 3.21-3.29 (m, 1 H) 3.30-3.37 (m, 1 H) 3.63-3.69 (m, 1 H) 3.71-3.79 (m, 4 H) 3.80- 4.04 (m, 5 H) 4.07-4.18 (m, 2 H) 4.18-4.34 (m, 2 H) 4.72-4.94 (m, 1 H) 6.86-6.91 (m, 1 H) 6.92-7.02 (m, 2 H) 7.07-7.11 (m, 1 H) 7.17-7.23 (m, 1 H) 7.66-7.73 (m, 1 H) 7.93- 8.26 (m, 1 H); LCMS confirms the MW (Rₜ: 1.13, MW: 797.00, [M + H]⁺ 778, Method 1). |
| 85 | 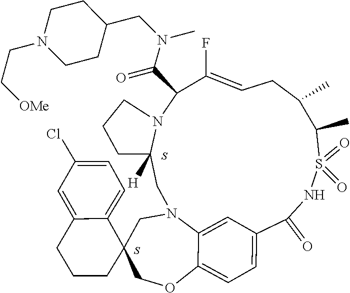 | ¹H NMR δ ppm 1.00-1.07 (m, 3 H) 1.39-1.43 (m, 1 H) 1.54-1.91 (m, 9 H) 1.95-2.13 (m, 6 H) 2.27-2.40 (m, 2 H) 2.53-2.71 (m, 2 H) 2.72-2.82 (m, 3 H) 2.87-3.05 (m, 5 H) 3.09- 3.26 (m, 6 H) 3.31-3.40 (m, 3 H) 3.45-3.77 (m, 6 H) 3.81-3.91 (m, 1 H) 3.92-4.02 (m, 2 H) 4.11-4.20 (m, 1 H) 4.41-4.69 (m, 1 H) 4.84-5.04 (m, 1 H) 6.85-6.98 (m, 2 H) 7.02- 7.11 (m, 2 H) 7.16-7.23 (m, 1 H) 7.29-7.35 (m, 1 H) 7.65-7.78 (m, 1 H); LCMS confirms the MW (Rₜ: 2.11, MW: 827.40, [M + H]⁺ 828, Method 3). |
| 86 | 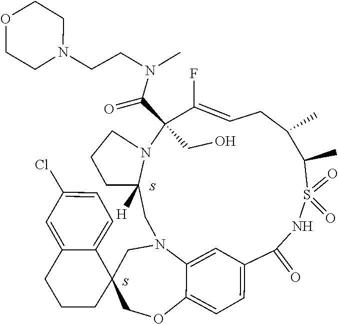 | ¹H NMR (400 MHz, CHLORO- FORM-d) δ ppm 1.02-1.10 (m, 3 H) 1.34-1.43 (m, 4 H) 1.46-1.55 (m, 1 H) 1.59-1.67 (m, 2 H) 1.72-1.84 (m, 1 H) 1.86-1.97 (m, 2 H) 2.01-2.15 (m, 4 H) 2.42-2.72 (m, 7 H) 2.72- 2.80 (m, 2 H) 2.86-3.01 (m, 3 H) 3.03-3.11 (m, 1 H) 3.13-3.19 (m, 2 H) 3.21-3.32 (m, 1 H) 3.33-3.49 (m, 1 H) 3.57-3.73 (m, 5 H) 3.80-4.00 (m, 4 H) 4.00-4.08 (m, 1 H) 4.13- 4.21 (m, 1 H) 4.46-4.64 (m, 1 H) 4.64-4.75 (m, 1 H) 6.88-6.93 (m, 1 H) 6.96-7.02 (m, 1 H) 7.06-7.09 (m, 1 H) 7.13-7.22 (m, 2 H) 7.67- 7.75 (m, 1 H); LCMS confirms the MW (Rₜ: 1.93, MW: 815.30, [M + H]⁺ 816, Method 3). |

Compound 87

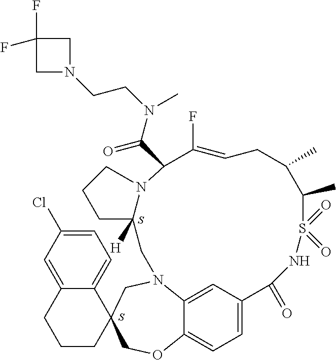

Intermediate 21 (112 mg, 0.157 mmol) and 3,3-difluoroazetidine (43.7 mg, 0.47 mmol) were stirred in 5 mL of anhydrous DCM and 1 mL of AcOH at room temperature during 1 h. Then, the mixture was cooled to 0° C., followed by portionwise addition of NaBH₃CN (29.52 mg, 0.47 mmol). The mixture was allowed to warm to room temperature and stirred 20 min.

Upon completion, the mixture was carefully quenched with 1 M NaOH sol. until pH reached ca 8-9. This was diluted with DCM and the organic layer was separated. The aqueous layer was back-extracted with DCM. Water was added to the organic layers and the mixture was carefully acidified until pH reached ca 5-6 with 1 M HCl sol. The organic layer was further separated, washed with brine (20 mL), dried (MgSO₄), filtered off and evaporated under reduced pressure.

A purification was performed via Prep HPLC (Stationary phase: RP XBridge Prep C18 OBD-5 μm, 50×250 mm, Mobile phase: 0.5% NH₄OAc solution in water+10% CH₃CN, CH₃CN). The pure fractions were collected and acetonitrile was evaporated under reduced pressure. DCM was added. The organic layer was extracted and the aqueous layer was back-extracted with DCM (×3). The combined dried (MgSO₄) organic layers were evaporated under reduced pressure to get Compound 87 (22.5 mg, yield 18%) as a white solid.

¹H NMR δ 1.05 (br d, J=6.2 Hz, 3H) 1.43 (br dd, J=7.2, 3.0 Hz, 3H) 1.70 (br d, J=5.1 Hz, 2H) 1.60-1.67 (m, 1H) 1.87-1.92 (m, 1H) 1.93-2.13 (m, 3H) 1.98-2.05 (m, 1H) 2.36 (br dd, J=14.4, 8.9 Hz, 1H) 2.74-2.80 (m, 1H) 2.74-2.82 (m, 2H) 2.89-2.98 (m, 2H) 2.94-3.03 (m, 1H) 3.06-3.23 (m, 2H) 3.29 (s, 1H) 3.30 (br s, 1H) 3.30-3.36 (m, 1H) 3.31-3.38 (m, 1H) 3.47-3.53 (m, 1H) 3.63 (t, J=12.0 Hz, 2H) 3.67-3.77 (m, 1H) 3.93 (br s, 1H) 3.97 (br d, J=12.1 Hz, 1H) 4.01 (br d, J=3.7 Hz, 1H) 4.20 (s, 1H) 4.13-4.18 (m, 1H) 4.47-5.21 (m, 1H) 4.64-5.00 (m, 1H) 6.88-6.98 (m, 2H) 7.02 (s, 1H) 7.06-7.12 (m, 1H) 7.20 (br d, J=8.4 Hz, 1H) 7.69 (d, J=8.4 Hz, 1H); LCMS confirms the MW (Rₜ: 2.18, MW: 791.00, [M+H]⁺ 792, Method 3); SFC (Rₜ 6.24, 2.31% isomer 1), (Rₜ 6.66, 98% isomer 2) (Rₜ: 6.66, Area %: 98, MW: 791.31, [M+H]⁺ 792, Method 15).

Compound 88

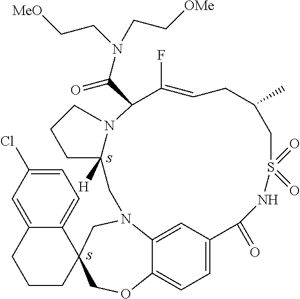

DBU (CAS [6674-22-2], 200 μL, 1.34 mmol) was added to a stirred solution of Intermediate 41 (100 mg, 0.128 mmol) in 15 mL of anhydrous DMF. The resulting mixture was stirred at room temperature for 5 min, followed by addition of diethyl cyanophosphonate (CAS [2942-58-7], 60 μL, 0.401 mmol). After 10 min, the mixture was diluted with EtOAc and quenched with water. The organic layer was extracted, washed with brine (×3), dried (MgSO₄), filtered off and evaporated under reduced pressure.

A first purification was performed via Prep HPLC (Stationary phase: RP XBridge Prep C18 OBD-10 μm, 30×150 mm, Mobile phase: 0.5% NH₄OAc solution in water+10% CH₃CN, CH₃CN). The purest fractions were collected, acetonitrile was evaporated and DCM and water were added. The organic layer was separated and the aqueous layer was washed with DCM (×3). The combined dried organic layer were evaporated under reduced pressure. A small column chromatography on silica gel with DCM/MeOH (1:0 to 97:3) was performed on the previous material to afford Compound 88 (34 mg, yield 35%) as a white solid.

¹H NMR (400 MHz, CHLOROFORM-d) δ ppm 1.20 (d, J=6.5 Hz, 3H) 1.36-1.46 (m, 1H) 1.63-1.74 (m, 3H) 1.78-1.90 (m, 2H) 1.92-2.02 (m, 3H) 2.04-2.10 (m, 1H) 2.42 (br dd, J=14.5, 7.7 Hz, 1H) 2.70-2.83 (m, 2H) 2.83-3.04 (m, 3H) 3.04-3.14 (m, 1H) 3.24-3.30 (m, 1H) 3.34 (s, 4H) 3.43 (s, 4H) 3.55-3.63 (m, 4H) 3.64-3.75 (m, 2H) 3.92-4.09 (m, 3H) 4.11-4.23 (m, 3H) 4.79-4.96 (m, 1H) 5.09 (br d, J=24.4 Hz, 1H) 6.86-6.96 (m, 2H) 7.00-7.08 (m, 1H) 7.09 (d, J=2.2 Hz, 1H) 7.21 (dd, J=8.5, 2.3 Hz, 1H) 7.70 (d, J=8.5 Hz, 1H). LCMS confirms the MW (Rₜ: 2.12, Area %: 100.00, MW: 760.00, [M+H]+ 761, Method 3).

Compounds 89 and 90

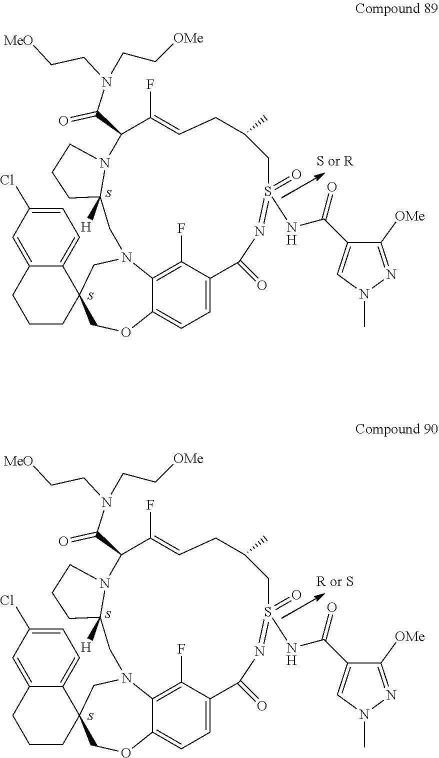

Intermediate 45 (250 mg, 0.329 mmol), 3-methoxy-1-methyl-1H-pyrazole-4-carboxylic acid (CAS [113100-56-4], 73.5 mg, 0.471 mmol) and Et₃N (0.457 mL, 3.288 mmol) were dissolved in 5 mL of anhydrous DCM, followed by addition of EDCI (CAS [25952-53-8], 147.0 mg, 0.767 mmol) and DMAP (88.2 mg, 0.722 mmol). The resulting mixture was stirred during 3 d at room temperature.

The mixture was evaporated under reduced pressure and the crude was subjected to a purification via Prep HPLC (Stationary phase: RP XBridge Prep C18 OBD-10 μm, 30×150 mm, Mobile phase: 0.5% NH₄OAc solution in water+10% CH₃CN, CH₃CN). The purest fractions were collected and acetonitrile was evaporated under reduced pressure. DCM and water were added. The organic layer was separated and the aqueous layer was back-extracted with DCM (×3). The combined dried (MgSO₄) organic layers were evaporated under reduced pressure to the diastereomeric mixture (23 mg) as a sticky oil. A purification was performed via Prep SFC (Stationary phase: Chiralcel Daicel IH 20×250 mm, Mobile phase: CO₂, EtOH-iPrOH (50-50)+0.4% iPrNH₂) yielding Compound 89 (14.3 mg, yield 5%) as a white solid and Compound 90 (7.5 mg, yield 3%) as an off-white solid.

Compound 89: ¹H NMR (400 MHz, CHLOROFORM-d) δ ppm 1.11 (br d, J=6.8 Hz, 3 H) 1.34-1.43 (m, 1H) 1.69 (br s, 3H) 1.81-1.98 (m, 3H) 2.01-2.10 (m, 1H) 2.16-2.27 (m, 1H) 2.28-2.40 (m, 1H) 2.50 (br dd, J=14.3, 7.3 Hz, 1H) 2.69-2.82 (m, 2H) 2.92 (br dd, J=14.7, 10.8 Hz, 1H) 3.14-3.22 (m, 1H) 3.34 (d, J=4.8 Hz, 6H) 3.35-3.42 (m, 2H) 3.49-3.60 (m, 6H) 3.60-3.64 (m, 1H)3.70 (s, 3H)3.72-3.78 (m, 2H) 3.81-3.93 (m, 4H) 3.96 (s, 3H) 4.04 (br d, J=14.2 Hz, 1H) 4.15 (d, J=12.1 Hz, 1H) 4.84 (br d, J=20.3 Hz, 1H) 5.58-5.76 (m, 1H) 6.85 (d, J=8.3 Hz, 1H) 7.08 (d, J=2.2 Hz, 1H) 7.20 (dd, J=8.5, 2.3 Hz, 1H) 7.31 (br d, J=8.3 Hz, 1H) 7.33-7.37 (m, 1H) 7.72-7.74 (m, 1H) 7.75-7.79 (m, 1H); LCMS confirms the MW (Rₜ: 2.04, Area %: 100.00, MW: 897.4, [M+H] 898.5, Method 4).

Compound 90: ¹H NMR (400 MHz, CHLOROFORM-d) δ ppm 1.04-1.14 (m, 3H) 1.37-1.45 (m, 1H)_{1.56}-1.77 (m, 4H)_{1.85} (br s, 4H)2.00-2.26 (m, 4H)2.30-2.41 (m, 1H) 2.72-2.83 (m, 2H) 2.84-3.04 (m, 3H) 3.13-3.22 (m, 1H) 3.27-3.39 (m, 8H) 3.39-3.50 (m, 2H) 3.72-3.76 (m, 3H) 3.86 (br s, 4H) 3.91-3.98 (m, 2H) 3.99-4.04 (m, 3H) 4.04-4.10 (m, 1H) 4.12-4.21 (m, 1H) 4.88-5.03 (m, 1H) 5.18-5.39 (m, 1H) 6.83-6.93 (m, 1H) 7.06-7.11 (m, 1H) 7.17-7.23 (m, 1H) 7.24-7.27 (m, 1H) 7.31-7.44 (m, 1H) 7.70-7.85 (m, 2H); LCMS confirms the MW (Rₜ: 2.01, Area %: 100.00, MW: 897.4, [M+H]⁺ 898.5, Method 4).

Analytical Methods

LCMS

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.

TABLE
LCMS Method Codes

| Method | | | | | Flow (mL/min) | Run time |
|---|---|---|---|---|---|---|
| Code | Instrument | column | mobile phase | gradient | Col T (° C.) | (min) |
| 1 | Waters: Acquity ® | BEH C18 column | A: 10 mM CH₃COONH₄ | 95% A and 5% B to 5% | 0.8 | 2 |
| | UPLC ® - | (1.7 μm, 2.1 × 50 mm; | in 95% H₂O + 5% CH₃CN | A and 95% B in 1.3 minutes | 55 | |
| | DAD and SQD | Waters Acquity) | B: CH₃CN | and hold for 0.7 minutes | | |
| 2 | Waters: Acquity ® | Waters: BEH | A: 0.1% NH₄HCO₃ | From 100% A to 5% A in | 0.8 | 2.0 |
| | UPLC ® - | (1.8 μm, 2.1*50 mm) | in 95% H₂O + 5% CH₃CN | 1.3 min, hold 0.7 min | 55 | |
| | DAD and SQD | | B: CH₃CN | | | |
| 3 | Waters: Acquity ® | Waters: BEH | A: 10 mM CH₃COONH₄ | From 100% A to 5% A in | 0.6 | 3.5 |
| | UPLC ® - | (1.8 μm, 2.1*100 mm) | in 95% H₂O + 5% CH₃CN | 2.10 min, to 0% A in 0.9 min, | 55 | |
| | DAD and SQD | | B: MeOH | to 5% A in 0.5 min | | |
| 4 | Waters: Acquity ® | Waters: BEH | A: 0.1% NH₄HCO₃ | From 100% A to 5% A in | 0.6 | 3.5 |
| | UPLC ® - | (1.8 μm, 2.1*100 mm) | in 95% H₂O + 5% CH₃CN | 2.10 min, to 0% A in 0.90 min, | 55 | |
| | DAD and SQD | | B: MeOH | to 5% A in 0.5 min | | |
| 5 | Waters: Acquity ® | Waters: BEH | A: 10 mM NH₄HCO₃ | From 100% A to 5% A in | 0.6 | 3.5 |
| | UPLC ® - | (1.8 μm, 2.1*100 mm) | in 95% H₂O + 5% CH₃CN | 2.10 min, to 0% A in 1.4 min | 55 | |
| | DAD and SQD2 | | B: MeOH | | | |
| 6 | Waters: Acquity ® | Waters: BEH | A: 0.1% NH₄HCO₃ | From 100% A to 5% A in | 0.6 | 3.5 |
| | UPLC ® - | (1.8 μm, 2.1*100 mm) | in 95% H₂O + 5% CH₃CN | 2.10 min, to 0% A in 0.9 min, | 55 | |
| | DAD and SQD | | B: CH₃CN | to 5% A in 0.5 min | | |
| 7 | Waters: Acquity ® | Waters: BEH | A: 10 mM NH₄HCO₃ | From 100% A to 5% A in | 0.6 | 3.5 |
| | UPLC ® - | (1.8 μm, 2.1*100 mm) | in 95% H₂O + 5% CH₃CN | 2.10 min, to 0% A in 0.9 min, | 55 | |
| | DAD and SQD3 | | B: CH₃CN | to 5% A in 0.4 min | | |
| 8 | Waters: Acquity ® | Waters: BEH | A: 0.1% HCOOH + 5% | From 100% A to 5% A in | 0.6 | 3.5 |
| | UPLC ® - | (1.8 μm, 2.1*100 mm) | CH₃OH in H₂O | 2.10 min, to 0% A in 0.9 min, | 55 | |
| | DAD and SQD | | B: CH₃CN | to 5% A in 0.5 min | | |
| 9 | Waters: Acquity ® | Waters: BEH | A: 10 mM CH₃COONH₄ | From 100% A to 5% A in | 0.6 | 3.5 |
| | UPLC ® - | (1.8 μm, 2.1*100 mm) | in 95% H2O + 5% CH₃CN | 2.10 min, to 0% A in 0.9 min, | 55 | |
| | DAD and SQD | | B: MeOH | to 5% A in 0.5 min | | |

SFC-MS methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Analytical SFC-MS Methods (Flow expressed in mL/min; column temperature (Col T) in ° C.; Run time in minutes, Backpressure (BPR) in bars; “iPrNH₂” means isopropylamine; “iPrOH” means 2-propanol; “EtOH” means ethanol; “min” mean minutes.

TABLE
Analytical SFC-MS Methods

| | | | | Flow | Run |
|---|---|---|---|---|---|
| | | | | (mL/ | time |
| | | | | min) | (min) |
| SFC | | mobile | | Col T | BPR |
| Method | Column | phase | gradient | (° C.) | (bars) |
| 11 | Daicel | A: CO₂ | 10%-50% B | 2.5 | 9.5 |
| | Chiralpak ® AD3 | B: | in 6 min, | 40 | 130 |
| | column (3.0 μm, | EtOH + 0.2% | hold 3.5 | | |
| | 150 × 4.6 mm) | iPrNH₂ | min | | |
| 12 | Daicel | A: CO₂ | 10%-50% B | 2.5 | 9.5 |
| | Chiralpak ® AS3 | B: | in 6 min, | 40 | 130 |
| | column (3.0 μm, | EtOH + 0.2% | hold 3.5 | | |
| | 150 × 4.6 mm) | iPrNH₂ | min | | |
| 13 | Daicel | A: CO₂ | 10%-50% B | 2.5 | 9.5 |
| | Chiralpak ® OJ3 | B: | in 6 min, | 40 | 130 |
| | column (3.0 μm, | EtOH + 0.2% | hold 3.5 | | |
| | 150 × 4.6 mm) | iPrNH₂ | min | | |
| 14 | Daicel | A: CO₂ | 10%-50% B | 2.5 | 9.5 |
| | Chiralpak ® OJ3 | B: | in 6 min, | 40 | 130 |
| | column (3.0 μm, | EtOH + 0.2% | hold 3.5 | | |
| | 150 × 4.6 mm) | iPrNH₂ | min | | |
| 15 | Daicel | A: CO₂ | 10%-50% B | 2.5 | 9.5 |
| | Chiralpak ® IC3 | B: | in 6 min, | 40 | 130 |
| | column (3.0 μm, | EtOH + 0.2% | hold 3.5 | | |
| | 150 × 4.6 mm) | iPrNH₂ | min | | |
| 16 | Daicel | A: CO₂ | 10%-50% B | 2.5 | 9.5 |
| | Chiralpak ® AD3 | B: | in 6 min, | 40 | 130 |
| | column (3.0 μm, | iPrOH + 0.2% | hold 3.5 | | |
| | 150 × 4.6 mm) | iPrNH₂ | min | | |
| 17 | REGIS Whelk ®- | A: CO₂ | 10%-50% B | 2.5 | 9.5 |
| | O-(R,R) column | B: | in 6 min, | 40 | 130 |
| | (3.5 μm, 150 × | EtOH + 0.2% | hold 3.5 | | |
| | 4.6 mm) | iPrNH₂ | min | | |

NMR

¹H NMR spectra were recorded on Bruker Avance III and Avance NEO 400 MHz spectrometers. CDCl₃ was used as solvent, unless otherwise mentioned. The chemical shifts are expressed in ppm relative to tetramethylsilane.

Pharmacological Analysis

### Biological Example 1

Terbium labeled Myeloid Cell Leukemia 1(Mcl-1) homogeneous time-resolved fluorescence (HTRF) binding assay utilizing the BIM BH3 peptide (H₂N—(C/Cy5Mal) WIAQELRRIGDEFN-OH) as the binding partner for Mcl-1.

Apoptosis, or programmed cell death, ensures normal tissue homeostasis, and its dysregulation can lead to several human pathologies, including cancer. Whilst the extrinsic apoptosis pathway is initiated through the activation of cell-surface receptors, the intrinsic apoptosis pathway occurs at the mitochondrial outer membrane and is governed by the binding interactions between pro- and anti-apoptotic Bcl-2 family proteins, including Mcl-1. In many cancers, the anti-apoptotic Bcl-2 protein(s), such as the Mcl-1, are upregulated, and in this way the cancer cells can evade apoptosis. Thus, inhibition of the Bcl-2 protein(s), such as Mcl-1, may lead to apoptosis in cancer cells, providing a method for the treatment of said cancers.

This assay evaluated inhibition of the BH3 domain: Mcl-1 interaction by measuring the displacement of Cy5-labeled BIM BH3 peptide (H₂N—(C/Cy5Mal) WIAQELRRIGDEFN-OH) in the HTRF assay format.

Assay Procedure

The following assay and stock buffers were prepared for use in the assay: (a) Stock buffer: 10 mM Tris-HCl, pH=7.5+150 mM NaCl, filtered, sterilized, and stored at 4° C.; and (b) 1X assay buffer, where the following ingredients were added fresh to stock buffer: 2 mM dithiothreitol (DTT), 0.0025% Tween-20, 0.1 mg/mL bovine serum albumin (BSA). The 1X Tb-Mcl-1+Cy5 Bim peptide solution was prepared by diluting the protein stock solution using the 1X assay buffer (b) to 25 pM Tb-Mcl-1 and 8 nM Cy5 Bim peptide.

Using the Acoustic ECHO, 100 nL of 100x test compound(s) were dispensed into individual wells of a white 384-well Perkin Elmer Proxiplate, for a final compound concentration of 1× and final DMSO concentration of 1%. Inhibitor control and neutral control (NC, 100 nL of 100% DMSO) were stamped into columns 23 and 24 of assay plate, respectively. Into each well of the plate was then dispensed 10 μL of the 1X Tb-Mcl-1+Cy5 Bim peptide solution. The plate was centrifuged with a cover plate at 1000 rpm for 1 minute, then incubated for 60 minutes at room temperature with plates covered.

The TR-FRET signal was read on an BMG PHERAStar FSX MicroPlate Reader at room temperature using the HTRF optic module (HTRF: excitation: 337 nm, light source: laser, emission A: 665 nm, emission B: 620 nm, integration start: 60 μs, integration time: 400 is).

Data Analysis

The BMG PHERAStar FSX MicroPlate Reader was used to measure fluorescence intensity at two emission wavelengths—665 nm and 620 nm—and report relative fluorescence units (RFU) for both emissions, as well as a ratio of the emissions (665 nm/620 nm)*10,000. The RFU values were normalized to percent inhibition as follows:

% inhibition=(((NC−IC)−(compound−IC))/(NC−IC))*100

where IC (inhibitor control, low signal)= mean signal of 1X Tb-MCl-1+Cy5 Bim peptide+inhibitor control or 100% inhibition of Mcl-1; NC (neutral control, high signal)= mean signal 1X Tb-MCl-1+Cy5 Bim peptide with DMSO only or 0% inhibition An 11-point dose response curve was generated to determine IC₅₀ values (using GenData) based on the following equation:

Y=Bottom+(Top−Bottom)/(1+10∧((log IC₅₀−X)*HillSlope))

where Y=% inhibition in the presence of X inhibitor concentration; Top=100% inhibition derived from the IC (mean signal of Mcl-1+inhibitor control); Bottom=0% inhibition derived from the NC (mean signal of Mcl-1+DMSO); Hillslope=Hill coefficient; and IC₅₀=concentration of compound with 50% inhibition in relation to top/neutral control (NC).

Kᵢ=IC₅₀/(1+[L]/Kd)

In this assay [L]=8 nM and Kd=10 nM

Representative compounds of the present invention were tested according to the procedure as described above, with results as listed in the Table below (n.d. means not determined).

| Compound | Tb-MCL1 Kᵢ (nM) |
|---|---|
| 1 | 0.027 |
| 2 | 0.089 |
| 3 | 0.129 |
| 4 | 0.084 |
| 5 | 0.026 |
| 6 | n.d. |
| 7 | 0.223 |
| 8 | 0.074 |
| 9 | 0.130 |
| 10 | 0.227 |
| 11 | 0.032 |
| 12 | 0.069 |
| 13 | 0.059 |
| 14 | 0.126 |
| 15 | 0.162 |
| 16 | 0.446 |
| 17 | 0.032 |
| 18 | 0.050 |
| 19 | 0.045 |
| 20 | 0.255 |
| 21 | 0.075 |
| 22 | 0.047 |
| 23 | 0.054 |
| 24 | 0.068 |
| 25 | 0.072 |
| 26 | 0.115 |
| 27 | 0.054 |
| 28 | 0.465 |
| 29 | 0.494 |
| 30 | 0.379 |
| 31 | 0.105 |
| 32 | 0.603 |
| 33 | 0.195 |
| 34 | 0.077 |
| 35 | 0.066 |
| 36 | 0.325 |
| 37 | 0.061 |
| 38 | 0.051 |
| 39 | 0.081 |
| 40 | 0.623 |
| 41 | 0.034 |
| 42 | 0.374 |
| 43 | 0.438 |
| 44 | 0.104 |
| 45 | 0.113 |
| 46 | 0.091 |
| 47 | 0.065 |
| 48 | 0.155 |
| 49 | 0.287 |
| 50 | 0.146 |
| 51 | 0.040 |
| 52 | 0.036 |
| 53 | 0.069 |
| 54 | 0.042 |
| 55 | 0.037 |
| 56 | 0.038 |
| 57 | 0.051 |
| 58 | 0.041 |
| 59 | 0.060 |
| 60 | 0.022 |
| 61 | 0.074 |
| 62 | 0.058 |
| 63 | 0.041 |
| 64 | 0.083 |
| 65 | 0.062 |
| 66 | 0.025 |
| 67 | 0.037 |
| 68 | 0.041 |
| 69 | 0.049 |
| 70 | 0.028 |
| 71 | 0.066 |
| 72 | 0.061 |
| 73 | 0.021 |
| 74 | 0.020 |
| 75 | 0.022 |
| 76 | 0.017 |
| 77 | 0.027 |
| 78 | 0.016 |
| 79 | 0.038 |
| 80 | 0.079 |
| 81 | 0.068 |
| 82 | 0.025 |
| 83 | 0.061 |
| 84 | 0.040 |
| 85 | 0.025 |
| 86 | 0.032 |
| 87 | 0.071 |
| 88 | 0.029 |
| 89 | 0.036 |
| 90 | 0.026 |

### Biological Example 2

MCL-1 is a regulator of apoptosis and is highly over-expressed in tumor cells that escape cell death. The assay evaluates the cellular potency of small-molecule compounds targeting regulators of the apoptosis pathway, primarily MCL-1, Bfl-1, Bcl-2, and other proteins of the Bcl-2 family. Protein-protein inhibitors disrupting the interaction of anti-apoptotic regulators with BH3-domain proteins initiate apoptosis.

The Caspase-Glo® 3/7 Assay is a luminescent assay that measures caspase-3 and −7 activities in purified enzyme preparations or cultures of adherent or suspension cells. The assay provides a proluminescent caspase-3/7 substrate, which contains the tetrapeptide sequence DEVD. This substrate is cleaved to release aminoluciferin, a substrate of luciferase used in the production of light. Addition of the single Caspase-Glo® 3/7 Reagent in an “add-mix-measure” format results in cell lysis, followed by caspase cleavage of the substrate and generation of a “glow-type” luminescent signal.

This assay uses the MOLP-8 human multiple myeloma cell line, which is sensitive to MCL-1 inhibition.

Materials:
Perkin Elmer Envision
Multidrop 384 and small volume dispensing cassettes
Centrifuge
Countess automated cell counter
Countess counting chamber slides
Assay plate: ProxiPlate-384 Plus, White 384-shallow well Microplate
Sealing tape: Topseal A plus
T175 culture flask

| | | |
|---|---|---|
| Product | Units | Storage |

| | | | |
|---|---|---|---|
| RPMI1640 (no L-Glutamine, no | 500 | mL | 4° C. |
| phenol red) | | | |
| Foetal Bovine Serum (FBS) (Heat | 500 | mL | 4° C. |
| inactivated) | | | |
| L-Glutamine (200 mM) | 100 | mL | −20° C. |
| Gentamicin (50 mg/mL) | 100 | mL | 4° C. |
| Caspase 3/7 Detection kit | 100 | mL | −20° C. |
| | 10 × 10 | mL | |

Cell Culture Media:

| MOLP8 | | |
|---|---|---|
| RPMI-1640 medium | 500 | mL |
| 20% FBS (heat inactivated) | 120 | mL |
| 2 mM L-Glutamine | 6.2 | mL |
| 50 μg/mL Gentamicin | 620 | μL |
| Assay media | | |
| RPMI-1640 medium | 500 | mL |
| 10% FBS (Heat inactivated) | 57 | mL |
| 2 mM L-Glutamine | 5.7 | mL |
| 50 μg/mL Gentamicin | 570 | μL |

Cell Culture:

Cell cultures were maintained between 0.2 and 2.0×10⁶ cells/mL. The cells were harvested by collection in 50 mL conical tubes. The cells were then pelleted at 500 g for 5 mins before removing supernatant and resuspension in fresh pre-warmed culture medium. The cells were counted and diluted as needed.

Caspase-Glo Reagent:

The assay reagent was prepared by transferring the buffer solution to the substrate vial and mixing. The solution may be stored for up to 1 week at 4° C. with negligible loss of signal.

Assay Procedure:

Compounds were delivered in assay-ready plates (Proxiplate) and stored at −20° C. Assays always include 1 reference compound plate containing reference compounds. The plates were spotted with 40 nL of compounds (0.5% DMSO final in cells; serial dilution; 30 pM highest conc. 1/3 dilution, 10 doses, duplicates). The compounds were used at room temperature and 4 μL of pre-warmed media was added to all wells except column 2 and 23. The negative control was prepared by adding 1% DMSO in media. The positive control was prepared by adding the appropriate positive control compound in final concentration of 60 pM in media. The plate was prepared by adding 4 μL negative control to column 23, 4 μL positive control to column 2 and 4 μL cell suspension to all wells in the plate. The plate with cells was then incubated at 37° C. for 2 hours. The assay signal reagent is the Caspase-Glo solution described above, and 8 μL was added to all wells. The plates were then sealed and measured after 30 minutes.

The activity of a test compound was calculated as percent change in apoptosis induction as follows:

$$\begin{matrix}LC = median of the Low Control values \\ = Central Reference in Screener \\ = DMSO \\ = 0 \%\end{matrix}$$

$$\begin{matrix}HC = Median of the High Control values \\ = Scale Reference in Screener \\ = 30 μ M of positive control \\ = 100 \% apoptosis induction\end{matrix}$$

% Effect (AC₅₀)=100−(sample-LC)/(HC-LC)*100

% Control=(sample/HC)*100

% Control min=(sample-LC)/(HC-LC)*100

Table: Measured AC₅₀ for Representative Compounds of Formula (I). Averaged values are reported over all runs on all batches of a particular compound.

| Compound | MOLP8 AC₅₀ (nM) |
|---|---|
| 1 | 80.1 |
| 2 | 89.2 |
| 3 | 99.2 |
| 4 | 71.3 |
| 5 | 111.2 |
| 6 | 84.3 |
| 7 | 346.4 |
| 8 | 141.0 |
| 9 | 179.8 |
| 10 | 285.2 |
| 11 | 163.2 |
| 12 | 196.0 |
| 13 | 296.7 |
| 14 | 265.8 |
| 15 | 435.0 |
| 16 | 445.5 |
| 17 | 234.2 |
| 18 | 213.2 |
| 19 | 79.3 |
| 20 | 193.0 |
| 21 | 78.0 |
| 22 | 56.7 |
| 23 | 101.2 |
| 24 | 151.3 |
| 25 | 154.3 |
| 26 | 153.3 |
| 27 | 395.9 |
| 28 | 301.6 |
| 29 | 408.5 |
| 30 | 350.7 |
| 31 | 238.4 |
| 32 | 477.5 |
| 33 | 218.1 |
| 34 | 76.9 |
| 35 | 90.1 |
| 36 | 398.7 |
| 37 | 49.5 |
| 38 | 187.0 |
| 39 | 19.7 |
| 40 | 138.3 |
| 41 | 99.1 |
| 42 | 463.3 |
| 43 | 399.0 |
| 44 | 87.3 |
| 45 | 113.2 |
| 46 | 328.6 |
| 47 | 240.9 |
| 48 | 112.5 |
| 49 | 366.0 |
| 50 | 174.8 |
| 51 | 78.3 |
| 52 | 90.5 |
| 53 | 169.0 |
| 54 | 76.4 |
| 55 | 81.2 |
| 56 | 89.4 |
| 57 | 84.4 |
| 58 | 55.7 |
| 59 | 33.4 |
| 60 | 92.3 |
| 61 | 166.0 |
| 62 | 134.1 |
| 63 | 57.4 |
| 64 | 74.7 |
| 65 | 56.7 |
| 66 | 357.0 |
| 67 | 119.5 |
| 68 | 102.7 |
| 69 | 117.8 |
| 70 | 41.9 |
| 71 | 94.2 |
| 72 | 75.2 |
| 73 | 91.4 |
| 74 | 435.8 |
| 75 | 83.0 |
| 76 | 36.7 |
| 77 | 143.5 |
| 78 | 546.6 |
| 79 | 105.5 |
| 80 | 133.5 |
| 81 | 537.4 |
| 82 | 556.7 |
| 83 | 252.5 |
| 84 | 317.0 |
| 85 | 759.3 |
| 86 | 1599.2 |
| 87 | 84.8 |
| 88 | 115.8 |
| 89 | 214.3 |
| 90 | 25.8 |

1. A compound of Formula (I): 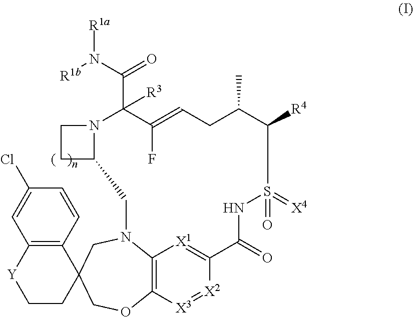
wherein:
R^{1a} and R^{1b} are each, independently, hydrogen, C₁₋₆ alkyl, C₂₋₆alkenyl, C₂₋₆alkynyl, C₃₋₇cycloalkyl, C₃₋₇cycloalkenyl, Het¹, Ar¹, Het², OR Cy¹,
wherein said C₁₋₆alkyl, C₂₋₆alkenyl, C₂₋₆alkynyl, C₃₋₇cycloalkyl or C₃₋₇cycloalkenyl is optionally substituted with one or two R²
or R^{1a} and R^{1b} are taken together to form together with the N-atom to which they are attached a 4- to 7-membered monocyclic fully saturated heterocyclyl containing one N-atom and optionally one additional heteroatom that is O, S, or N, wherein said S-atom is optionally substituted to form S(═O) or S(═O)₂, and wherein said heterocyclyl is optionally substituted with one or two substituents that are each, independently, oxo, OR^{f}, SR^{f}, NR^{d}R^{e}, CN, halo, CF₃, or C₁₋₄alkyl optionally substituted with one substituent that is OR^{f}, SR^{f}, CN or halo;
or R^{1a} and R^{1b} are taken together to form together with the N-atom to which they are attached a 6- to 11-membered bicyclic fully saturated heterocyclyl containing one N-atom and optionally one or two additional heteroatoms that are each, independently, O, S, or N, wherein said S-atom is optionally substituted to form S(═O) or S(═O)₂, and wherein said heterocyclyl is optionally substituted with one or two substituents that are each, independently, oxo, OR^{f}, SR^{f}, NR^{d}R^{e}, CN, halo, CF₃, or C₁₋₄alkyl optionally substituted with one substituent that is OR^{f}, SR^{f}, CN or halo;
each R² is independently OR^{f}, SR^{f}, CN, halo, CF₃, NR^{m}Rⁿ, SO₂R^{c}, C(═O)R^{C}, C(═O)OR^{d}, C(═O)NR^{d}R^{C}, SO₂NR^{d}R^{e}, C₃₋₇cycloalkyl, C₃₋₇ cycloalkenyl, Het¹, Ar¹, Het², or Cy¹,
wherein said C₃₋₇cycloalkyl or C₃₋₇cycloalkenyl is optionally substituted with one or two substituents that are each, independently, OR^{f}, SR^{f}, CN, halo or NR^{d}R^{e};
R^{c} is C₁₋₆alkyl, C₃₋₇cycloalkyl, Het¹, Ar¹ or Het²;
R^{m} and Rⁿ are each, independently, hydrogen, methyl, C₂₋₇alkyl, C₃₋₇cycloalkyl, Het¹, Ar¹, or Het², wherein said C₂₋₇alkyl or C₃₋₇ cycloalkyl is optionally substituted with one or two substituents that are each, independently, ORⁱ, SRⁱ, NR^{g}R^{h}, CN, halo, or C₁₋₄alkyl optionally substituted with one substituent that is ORⁱ, SRⁱ, CN, NR^{g}R^{h} or halo;
R^{d} and R^{c} are each, independently, hydrogen, methyl, C₂₋₇alkyl, C₃₋₇cycloalkyl, Het¹, Ar¹, or Het², wherein said C₂₋₇alkyl or C₃₋₇ cycloalkyl is optionally substituted with one or two substituents that are each, independently, ORⁱ, SRⁱ, NR^{g}R^{h}, CN, halo, or C₁₋₄alkyl optionally substituted with one substituent that is ORⁱ, SRⁱ, CN, NR^{g}R^{h} or halo;
or R^{d} and R^{e} are taken together to form together with the N-atom to which they are attached a 4- to 7-membered monocyclic fully saturated heterocyclyl containing one N-atom and optionally one additional heteroatom that is O, S, or N, wherein said S-atom is optionally substituted to form S(═O) or S(═O)₂, and wherein said heterocyclyl is optionally substituted with one or two substituents that are each, independently, ORⁱ, SRⁱ, NR^{g}R^{h}, CN, halo, CF₃, or C₁₋₄alkyl optionally substituted with one substituent that is ORⁱ, SRⁱ, CN or halo;
or R^{d} and R^{o} are taken together to form together with the N-atom to which they are attached a fused 6- to 11-membered bicyclic fully saturated heterocyclyl containing one N-atom and optionally one or two additional heteroatoms that are each, independently, O, S, or N, wherein said S-atom is optionally substituted to form S(═O) or S(═O)₂, and wherein said heterocyclyl is optionally substituted with one or two substituents that are each, independently, ORⁱ, SRⁱ, NR^{g}R^{h}, CN, halo, CF₃, or C₁₋₄ alkyl optionally substituted with one substituent that is ORⁱ, SRⁱ, CN or halo;
n is 1 or 2;
R^{f} is hydrogen, C₁₋₆alkyl, CF₃, C₃₋₇cycloalkyl, Het¹, Ar¹, or Het², wherein said C₁₋₆alkyl or C₃₋₇cycloalkyl is optionally substituted with one substituent that is ORⁱ, SRⁱ, CN, halo, NR^{m}Rⁿ, SO₂R^{c}, C(═O)R^{C}, C(═O)OR^{d}, C(═O)NR^{d}R^{e}, SO₂NR^{d}R^{e}, C₃₋₇cycloalkyl, Het¹, Ar¹ or Het²;
R^{g} and R^{h} are each, independently, hydrogen, C₁₋₆ alkyl or C₃₋₇cycloalkyl;
or R^{g} and R^{h} are taken together to form together with the N-atom to which they are attached a 4- to 7-membered monocyclic fully saturated heterocyclyl containing one N-atom and optionally one additional heteroatom that is O, S or N, wherein said S-atom is optionally substituted to form S(═O) or S(═O)₂, and
Het¹ is a 4- to 7-membered monocyclic fully saturated heterocyclyl containing one or two heteroatoms that are each, independently, O, S, or N, wherein said S-atom is optionally substituted to form S(═O) or S(═O)₂, and wherein said heterocyclyl is optionally substituted with one or two substituents that are each, independently, ORⁱ, SRⁱ, NR^{j}R^{k}, CN, halo, CF₃, or C₁₋₄alkyl optionally substituted with one substituent that is ORⁱ, SRⁱ, CN or halo;
Het¹ is a 5- to 6-membered monocyclic aromatic ring containing one, two, three or four heteroatoms that are each, independently, O, S, or N, wherein said S-atom is optionally substituted to form S(═O) or S(═O)₂, and wherein said aromatic ring is optionally substituted with one or two substituents that are each, independently, ORⁱ, SRⁱ, NR^{j}R^{k}, CN, halo, CF₃, or C₁₋₄alkyl optionally substituted with one substituent that is ORⁱ, SRⁱ, CN or halo;
Cy¹ is a 6- to 11-membered bicyclic fully saturated ring system optionally containing one or two heteroatoms that are each, independently, O, S, or N, wherein said S-atom is optionally substituted to form S(═O) or S(═O)₂, and wherein said ring system is optionally substituted with one or two substituents that are each, independently, Oi¹, SRⁱ, NR^{j}R^{k}, CN, halo, CF₃, or C₁₋₄alkyl optionally substituted with one substituent that is ORⁱ, SRⁱ, CN or halo;
Ar¹ is phenyl optionally substituted with one or two substituents that are each, independently, ORⁱ, SRⁱ, NR^{g}R^{h}, CN, halo, CF₃, or C₁₋₄alkyl optionally substituted with one substituent that is ORⁱ, SRⁱ, CN or halo;
Rⁱ is hydrogen, C₁₋₆alkyl or C₃₋₇cycloalkyl;
Rⁱ and R^{k} are each, independently, hydrogen, C₁₋₆ alkyl or C₃₋₇cycloalkyl:
R³ is hydrogen, C₁₋₄alkyl or C₁₋₄alkyl-OH;
R⁴ is hydrogen or methyl;
R⁵ is —(C═O)-phenyl, —(C═O)-Het⁴ or —(C═O)-Het³: wherein said phenyl, Het³ or Het⁴ are optionally substituted with one or two substituents that are methyl or methoxy;
Het⁴ is C-linked 4- to 7-membered monocyclic fully saturated heterocyclyl containing one or two heteroatoms that are each, independently, O, S, or N; wherein said S-atom is optionally substituted to form S(═O) or S(═O)₂;
Het³ is a C-linked 5-or 6-membered monocyclic aromatic ring containing one, two or three heteroatoms that are each, independently, O, S, or N;
Y is O or CH₂;
X¹ is CR⁶;
X⁴ is CR⁷;
X³ is CR⁸;
R⁶, R⁷ and R⁸ are each, independently, hydrogen, fluoro or chloro;
X⁴ is O or NR⁵;
or a pharmaceutically acceptable salt, or a solvate thereof.

2. The compound according to claim 1, wherein:
Het² is a 5- to 6-membered monocyclic aromatic ring containing one or two heteroatoms that are each, independently, O, S, or N, wherein said S-atom is optionally substituted to form S(═O) or S(═O)₂, and wherein said aromatic ring is optionally substituted with one or two substituents that are each, independently, ORⁱ, SRⁱ, NR^{j}R^{k}, CN, halo, CF₃, or C₁₋₄alkyl optionally substituted with one substituent that is ORⁱ, SRⁱ, CN or halo;
X¹ is CH;
X² is CH;
X³ is CH;
R³ is hydrogen;
R⁴ is methyl;
X⁴ is O.

3. The compound according to claim 1, wherein:
R^{1a} and R^{1b} are each, independently, C₁₋₆alkyl, Het¹, or Ar¹, wherein said C₁₋₆alkyl is optionally substituted with one or two R²;
or R^{1a} and R^{1b} are taken together to form together with the N-atom to which they are attached a 4- to 7-membered monocyclic fully saturated heterocyclyl containing one N-atom and optionally one additional heteroatom that is O, S, or N, wherein said heterocyclyl is optionally substituted with one or two substituents that are each, independently, OR^{f}, NR^{d}R^{e}, CN, halo, CF₃, or C₁₋₄alkyl optionally substituted with one substituent that is OR^{f} or CN;
or R^{1a} and R^{1b} are taken together to form together with the N-atom to which they are attached a 6- to 11-membered bicyclic fully saturated heterocyclyl containing one N-atom and optionally one or two additional heteroatoms that are each, independently, O, S, or N, wherein said heterocyclyl is optionally substituted with one or two substituents that are each, independently, halo or C₁₋₄alkyl;
each R² is, independently, OR^{f}, CF₃, NR^{m}Rⁿ, SO₂R^{c}, Het¹, or Het²,
R^{c} is C₁₋₆alkyl;
R^{m} and Rⁿ are each, independently, C₂₋₇alkyl optionally substituted with one or two ORⁱ substituents;
R^{d} and R^{e} are taken together to form together with the N-atom to which they are attached a 4- to 7-membered monocyclic fully saturated heterocyclyl containing one N-atom and optionally one additional heteroatom that is O, S, or N;
R^{f} is hydrogen, C₁₋₆alkyl, Het¹, or Het², wherein said C₁₋₆alkyl is optionally substituted with one ORⁱ substituent;
Het¹ is a 4- to 7-membered monocyclic fully saturated heterocyclyl containing one or two heteroatoms that are each, independently, O, S, or N, wherein said heterocyclyl is optionally substituted with one or two substituents that are each, independently, halo, CF₃, or C₁₋₄ alkyl optionally substituted with one ORⁱ substituent;
Het² is a 5- to 6-membered monocyclic aromatic ring containing one, two, three or four heteroatoms that are each, independently, O, S, or N, wherein said aromatic ring is optionally substituted with one or two substituents that are each, independently, halo or C₁₋₄alkyl;
Ar is phenyl;
Rⁱ is C₁₋₆alkyl;
R³ is hydrogen, or C₁₋₄alkyl-OH;
R⁵ is —(C═O)-Het³; wherein said Het³ is optionally substituted with one or two substituents that are methyl or methoxy;
R⁶, R⁷ and R⁸ are each, independently, hydrogen or fluoro.

4. The compound according to claim 1, wherein
R^{1a} and R^{1b} are each, independently, C₁₋₆alkyl, Ar¹, or Cy¹,
wherein said C₁₋₆alkyl is optionally substituted with one R²;
or R^{1a} and R^{1b} are taken together to form together with the N-atom to which they are attached a 4- to 7-membered monocyclic fully saturated heterocyclyl containing one N-atom and optionally one additional heteroatom that is O, S, or N, wherein said S-atom is optionally substituted to form S(═O) or S(═O)₂, and wherein said heterocyclyl is optionally substituted with one or two substituents that are each, independently, OR^{f}, CF₃, or C₁₋₄ alkyl optionally substituted with one OR^{f};
or R^{1a} and R^{1b} are taken together to form together with the N-atom to which they are attached a 6- to 11-membered bicyclic fully saturated heterocyclyl containing one N-atom and optionally one or two additional heteroatoms that are each, independently, O, S, or N, wherein said S-atom is optionally substituted to form S(═O) or S(═O)₂;
R² is OR^{f}, CF₃, Het¹, or Het²;
n is 1 or 2,
R^{f} is hydrogen, C₁₋₆alkyl, Het¹, or C₁₋₆alkyl substituted with one OR¹;
Het¹ is a 4- to 7-membered monocyclic fully saturated heterocyclyl containing one or two heteroatoms that are each, independently, O, S, or N, wherein said S-atom might-bis optionally substituted to form S(═O) or S(═O)₂, and wherein said heterocyclyl is optionally substituted with one or two halo;
Het² is a 5- to 6-membered monocyclic aromatic ring containing one or two heteroatoms that are each, independently, O, S, or N, wherein said S-atom is optionally substituted to form S(═O) or S(═O)₂, and wherein said aromatic ring is optionally substituted with one or two C₁₋₄alkyl;
Cy¹ is a 6- to 11-membered bicyclic fully saturated ring system optionally containing one or two heteroatoms that are each, independently, O, S, or N, wherein said S-atom is optionally substituted to form S(═O) or S(═O)₂, and wherein said ring system is optionally substituted with one or two halo;
Ar¹ is phenyl;
Rⁱ is C₁₋₆alkyl;
Y is CH₂.

5. The compound according to claim 1, wherein R^{1a} and R^{1b} are each, independently, C₁₋₆alkyl optionally substituted with one R².

6. The compound according to claim 1, wherein n is 2.

7. The compound according to claim 1, wherein Y is CH₂.

8. A pharmaceutical composition comprising a compound of claim 1 and a pharmaceutically acceptable carrier or diluent.

9. A process for preparing a pharmaceutical composition of claim 8, comprising mixing a pharmaceutically acceptable carrier with a therapeutically effective amount of the compound of claim 1.

10-11. (canceled)

12. The method of claim 13, wherein the cancer is prostate, lung, pancreatic, breast, ovarian, cervical, melanoma, B-cell chronic lymphocytic leukemia (CLL), acute myeloid leukemia (AML), or acute lymphoblastic leukemia (ALL).

13. A method of treating or preventing cancer, comprising administering to a subject in need thereof, a therapeutically effective amount of the compound of claim 1.

14. A method of treating or preventing cancer, comprising administering the pharmaceutical composition of claim 8 to a subject in need thereof.

15. The method of claim 14, wherein the cancer is prostate, lung, pancreatic, breast, ovarian, cervical, melanoma, B-cell chronic lymphocytic leukemia (CLL), acute myeloid leukemia (AML), or acute lymphoblastic leukemia (ALL).
